(19) United States
(12) Patent Application Publication
(10) Pub. No.: US 2015/0182667 A1
(43) Pub. Date: Jul. 2, 2015

(54) Composition with Biofilm Dispersal Agents

(71) Applicant: Vanderbilt University, Nashville, TN (US)

(72) Inventors: Scott A. Guelcher, Thompsons Station, TN (US); Joseph C. Wenke, La Vernia, TX (US); Carlos C. Sanchez, Jr., San Antonio, TX (US); Kevin S. Akers, San Antonio, TX (US); Chad A. Kruger, San Antonio, TX (US); Edna M. Prieto, Nashville, TN (US); Katarzyna Zienkiewicz, Nashville, TN (US)

(21) Appl. No.: 14/420,625

(22) PCT Filed: Aug. 8, 2013
(86) PCT No.: PCT/US13/54216

§ 371 Date: Feb. 9, 2015

Related U.S. Application Data
(60) Provisional application No. 61/681,026, filed on Aug. 8, 2012.

(51) Int. Cl. A61L 27/54; A61L 27/36; A61L 27/18

(52) U.S. Cl. CPC A61L 27/54; A61L 27/18; A61L 27/3608; A61L 2300/404

(57) ABSTRACT

Embodiments of the presently-disclosed subject matter provide composites that comprise a tissue graft and a biofilm dispersal agent. The tissue graft can be bone tissue graft, a soft tissue graft, or the like. In specific embodiments the tissue graft is a polyurethane graft and in other embodiments the tissue graft is bone particles, such as demineralized bone matrix. The biofilm dispersal agent can be one or more D-amino acids. The presently-disclosed subject matter further includes methods for treating tissue of a subject that comprise administering the present composites as well as methods for manufacturing the present composites.

### RELATED APPLICATIONS

This application claims priority from U.S. Provisional Application Ser. No. 61/681,026, filed Aug. 8, 2012, the entire disclosure of which is incorporated herein by this reference.

### GOVERNMENT INTEREST

This invention was made with government support under grant number W81XWH-07-1-0211 and intramural funding awarded by the Department of Defense. The government has certain rights in the invention.

### TECHNICAL FIELD

The presently-disclosed subject matter relates to composites comprising dispersal agents. In particular, embodiments of the presently-disclosed subject matter include composites comprising tissue grafts, D-amino acids, and, optionally, a bioactive agent. Embodiments of the presently-disclosed subject matter also include methods of utilizing and synthesizing the present composites.

### INTRODUCTION

Current approaches to bone healing require a two-step process in which the infection is first controlled by implantation of non-degradable tobramycin-impregnated poly(methylmethacrylate) (PMMA) beads, followed by implantation of a bone graft to promote bone healing. To reduce the healing time of the patient, it is desirable to promote bone fracture healing and control infection through one surgical procedure. Thus, conventional clinical practice removes devitalized tissue and gross contaminants with debridement and irrigation, for example, and the vast majority of the remaining bioburden is killed by systemic antibiotics. However, systemic antibiotics present significant disadvantages, including the potential for toxicity from systemic agents, difficulty in achieving high concentrations of antimicrobial agents at the site of infection, and problems with patient compliance. Moreover, systemic antibiotics are generally given for less than one week.

Furthermore, since the PMMA beads do not resorb and antibiotic elution decreases after two to six weeks, they require surgical removal prior to definitive grafting. In contrast, calcium sulfate pellets are biodegradable and can be mixed with antibiotics and used either as or with the definitive bone graft. However, calcium sulfate pellets have in some cases been associated with seromas and drainage problems, and can also cause nursing complications because the weeping wounds can be mistaken as being infected. Also, while osteogenic or osteoinductive grafts induce new bone formation, they are not protected from persistent bacteria remaining within the wound or from nosocomial or hematological introduction of the bacteria to the wound. Thus antibiotics can be administered to reduce the bacteria, preferably while the bacteria are in the solitary, nomadic planktonic stage. Otherwise, within about 5 to 10 hours of injury, the bacteria adhere to the surface of wounds and form biofilms.

Biofilms are an association of single and/or multiple bacterial species attached to a surface encased within a self-produced extracellular polymeric matrix (EPM) consisting of polysaccharides, protein and extracellular DNA, which constitutes a protected mode of growth. Compared to their planktonic counterparts, biofilm-derived bacteria have distinctive phenotypes, in terms of growth, gene expression, and protein production, resulting in an inherent resistance the action of antimicrobials, host mechanisms of clearance, and the ability to evade immune-detection allowing the biofilms to persist for extended periods within the host. Biofilm development is a highly coordinated and reversible process beginning with initial attachment and growth of cells on a surface culminating in the detachment or dispersal of cells into the surrounding environment. The detachment of cells from the biofilm into the environment is an essential stage of the biofilm life cycle contributing to bacterial survival and disease transmission. For both Gram-positive and Gram-negative microorganisms, coordination of biofilm formation and dispersal, as well as virulence, occurs through the detection of self-produced diffusible factors by quorum sensing systems.

Biofilm formation by bacterial pathogens is a major virulence factor associated with the development of a number of chronic infections, including otitis media, periodontitis, endocarditis, cystic fibrosis, and osteomyelitis, as well as most device-associated infections. Osteomyelitis, for example, is a debilitating disease, characterized by inflammatory destruction of bone lasting for weeks or developing into a chronic-persistent infection lasting for months to years. Infection is preceded by the local spread of bacteria, and less commonly fungi, from a contiguous contaminated source directly following trauma or as a result of hematogenous spread following bone surgery and joint replacement. Chronic osteomyelitis is an important source of patient morbidity, and is the primary reason for extremity amputation. Among the pathogenic microorganisms associated with chronic osteomyelitis, Staphylococcus aureus is the most frequent cause, accounting for >50% of all cases. Other bacterial species including Coagulase-negative staphylococci, Enterobacteriaceae, Acinetobacter baumannii, and Pseudomonas aeruginosa have also been reported.

Thus, bacterial signaling molecules that trigger the dispersal are desirable to treat chronic infections. Recent studies have shown that use of dispersal agents, including bismuth thiols, quorum sensing inhibitors, and recombinant DNAses, can enhance the effects of conventional antibiotics against biofilms and improve survival outcomes in animal models of chronic disease. However, major limitations to the applications of such therapies are usually the result of cellular cytotoxicity, such as with bismuth thiols, and more importantly the specificity of the dispersal agent for certain bacterial species.

Thus, there remains a need for novel composites that comprise tissue grafts and that do not have the same problems associated with grafts discussed above. There also remains a need for a graft that treats tissue, including bone tissue and/or soft tissue, and reduces or eliminates the bioburden at a wound site. Hence, there remains a need for grafts that are biodegradable, can eliminate organisms that are planktonic and that form biofilms, and can help treat tissue.

### SUMMARY

The presently-disclosed subject matter meets some or all of the above-identified needs, as will become evident to those of ordinary skill in the art after a study of information provided in this document.

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned, likewise, those features can be applied to other embodiments of the presently disclosed subject matter, whether listed in this Summary or not. To avoid excessive repetition, this Summary does not list or suggest all possible combinations of such features.

The presently-disclosed subject matter includes composites, where exemplary composites comprise a tissue graft and a biofilm dispersal agent. In this regard, there is no particular limitation on the manner in which the tissue graft and the biofilm dispersal agent are combined. In some embodiments the biofilm dispersal agent is on a surface of the tissue graft. In some embodiments the biofilm dispersal agent is within (e.g., impregnated) the tissue graft. Furthermore, in some embodiments the biofilm dispersal agent is a combination of the two, and is on the surface of a tissue graft as well as within the tissue graft.

Exemplary tissue grafts include bone tissue grafts, soft tissue grafts, including skin tissue grafts, or combinations thereof. The graft itself may comprise a polymer. For example, in some embodiments the tissue graft comprises a polymer that includes a polyisocyanate prepolymer, the polyisocyanate prepolymer including a first polyol and a polyisocyanate, and a second polyol. In some embodiments the first polyol includes poly(ethylene glycol) (PEG). Furthermore, in some embodiments the polyisocyanate includes an aliphatic polyisocyanate chosen from the group consisting of lysine methyl ester diisocyanate (LDI), lysine triisocyanate (LTI), 1,4-diisocyanatobutane (BDI), hexamethylene diisocyanate (HDI), dimers and trimers of HDI, and combinations therof.

In further embodiments the tissue graft includes a collagen sponge. In yet further embodiments the tissue graft includes a tissue allograft, a tissue autograft, a tissue xenograft, a tissue isograft, a synthetic tissue substitute, or a combination thereof. For example, some embodiments of tissue grafts include demineralized bone particles (demineralized bone matrix), mineralized bone particles, or a combination thereof.

With regard to the biofilm dispersal agent, several types and combinations of biofilm dispersal agents may comprise a composite. Exemplary composites can comprise about 0.001 wt % to about 20 wt % of a biofilm dispersal agent. In some embodiments the biofilm dispersal agent is selected from the group consisting of a D-amino acid, a polyamine, a recombinant DNase, a bismuth thiol, a fatty acid, cis-2-decenoic acid, tetradecanoic acid, 9-hexadecenoic acid, palmic acid, 9,12-linoleic acid, 9-oleic acid, 10-oleic acid, octadecoic acid, 7,10-oleic acid, 5,8,11,14-arachidonic acid, 7,10,13-eicosatrienoic acid, and combinations thereof.

Furthermore, if the biofilm dispersal agent is a D-amino acid, the D-amino acid can be selected from the group consisting of D-arginine, D-histidine, D-lysine, D-aspartic acid, D-glutamic acid, D-serine, D-threonine, D-asparagine, D-glutamine, D-cysteine, D-proline, D-alanine, D-valine, D-isoleucine, D-leucine, D-methionine, D-phenylalanine, D-tyrosine, D-tryptophan, and combinations thereof. Specific embodiments comprise a biofilm dispersal agent that includes at least two of D-phenylalanine, D-methionine, D-tryptophan, and D-proline.

Some composites can further comprise a biologically active agent (bioactive agent). The biologically active agent can be selected from the group consisting of enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, cells, viruses, virenos, virus vectors, prions, and combinations thereof. In specific embodiments the biologically active agent is selected from the group consisting of clindamycin, cefazolin, oxacillin, rifampin, trimethoprim/sulfamethoxazole, vancomycin, ceftazadime, ciprofloxacin, colistin, imipenem, and combinations thereof.

The presently-disclosed subject matter also includes methods for treating tissue that comprise administering embodiments of the present composites. In some embodiments the method comprises contacting a tissue site of a subject in need thereof with a composite, the composite including a tissue graft and a biofilm dispersal agent, wherein the biofilm dispersal agent can be on a surface of the tissue graft, within the tissue graft, or a combination thereof. In some embodiments, once the composite has been contacted with a tissue site, the composite can release the biofilm dispersal agent for up to 8 weeks or more.

The step of administering can include implanting, injecting, or pre-molding and contacting the composite on to a tissue site. Furthermore, the tissue site can include a bone tissue site, a soft tissue site, or a combination thereof. Thus, depending on the tissue graft that comprises the composite, administration methods can be tailored accordingly.

Furthermore, the presently-disclosed subject matter includes methods for manufacturing a composite. In some embodiments the methods comprise providing a tissue graft and then applying a biofilm dispersal agent on a surface of the tissue graft, within the tissue graft, or a combination thereof. In some embodiments the step of applying the biofilm dispersal agent comprising coating (e.g., spraying, laminating, etc.) the biofilm dispersal agent on a tissue graft. In other embodiments the tissue graft is a polymeric material, and the step of applying the biofilm dispersal agent includes curing a mixture of the polymeric material and the biofilm dispersal agent. In this regard, the biofilm dispersal agent used in the applying step can be a powder. The method can further comprise applying a biologically active agent to the tissue graft.

Further advantages of the presently-disclosed subject matter will become evident to those of ordinary skill in the art after a study of the description, Figures, and non-limiting Examples in this document.

### DEFINITIONS

The term “bioactive agent” is used herein to refer to compounds or entities that alter, promote, speed, prolong, inhibit, activate, or otherwise affect biological or chemical events in a subject (e.g., a human). For example, bioactive agents may include, but are not limited to osteogenic, osteoinductive, and osteoconductive agents, anti-HIV substances, anti-cancer substances, antibiotics, immunosuppressants, anti-viral agents, enzyme inhibitors, neurotoxins, opioids, hypnotics, anti-histamines, lubricants, tranquilizers, anti-convulsants, muscle relaxants, anti-Parkinson agents, anti-spasmodics and muscle contractants including channel blockers, miotics and anti-cholinergics, anti-glaucoma compounds, anti-parasite agents, anti-protozoal agents, and/or anti-fungal agents, modulators of cell-extracellular matrix interactions including cell growth inhibitors and anti-adhesion molecules, vasodilating agents, inhibitors of DNA, RNA, or protein synthesis, anti-hypertensives, analgesics, anti-pyretics, steroidal and non-steroidal anti-inflammatory agents, anti-angiogenic factors, angiogenic factors, anti-secretory factors, anticoagulants and/or antithrombotic agents, local anesthetics, ophthalmics, prostaglandins, anti-depressants, anti-psychotics, targeting agents, chemotactic factors, receptors, neurotransmitters, proteins, cell response modifiers, cells, peptides, polynucleotides, viruses, and vaccines. In certain embodiments, the bioactive agent is a drug. In certain embodiments, the bioactive agent is a small molecule.

A more complete listing of bioactive agents and specific drugs suitable for use in the present invention may be found in “Pharmaceutical Substances: Syntheses, Patents, Applications” by Axel Kleemann and Jurgen Engel, Thieme Medical Publishing, 1999; the “Merck Index: An Encyclopedia of Chemicals, Drugs, and Biologicals”, Edited by Susan Budavari et al., CRC Press, 1996, the United States Pharmacopeia-25/National Formulary-20, published by the United States Pharmcopeial Convention, Inc., Rockville Md., 2001, and the “Pharmazeutische Wirkstoffe”, edited by Von Keemann et al., Stuttgart/New York, 1987, all of which are incorporated herein by reference. Drugs for human use listed by the U.S. Food and Drug Administration (FDA) under 21 C.F.R. §§330.5, 331 through 361, and 440 through 460, and drugs for veterinary use listed by the FDA under 21 C.F.R. §§500 through 589, all of which are incorporated herein by reference, are also considered acceptable for use in accordance with the present invention.

The terms, “biodegradable”, “bioerodable”, or “resorbable” materials, as used herein, are intended to describe materials that degrade under physiological conditions to form a product that can be metabolized or excreted without damage to the subject. In certain embodiments, the product is metabolized or excreted without permanent damage to the subject. Biodegradable materials may be hydrolytically degradable, may require cellular and/or enzymatic action to fully degrade, or both. Biodegradable materials also include materials that are broken down within cells. Degradation may occur by hydrolysis, oxidation, enzymatic processes, phagocytosis, or other processes.

The term “biocompatible” as used herein, is intended to describe materials that, upon administration in vivo, do not induce undesirable side effects. In some embodiments, the material does not induce irreversible, undesirable side effects. In certain embodiments, a material is biocompatible if it does not induce long term undesirable side effects. In certain embodiments, the risks and benefits of administering a material are weighed in order to determine whether a material is sufficiently biocompatible to be administered to a subject.

The term “biomolecules” as used herein, refers to classes of molecules (e.g., proteins, amino acids, peptides, polynucleotides, nucleotides, carbohydrates, sugars, lipids, nucleoproteins, glycoproteins, lipoproteins, steroids, natural products, etc.) that are commonly found or produced in cells, whether the molecules themselves are naturally-occurring or artificially created (e.g., by synthetic or recombinant methods). For example, biomolecules include, but are not limited to, enzymes, receptors, glycosaminoglycans, neurotransmitters, hormones, cytokines, cell response modifiers such as growth factors and chemotactic factors, antibodies, vaccines, haptens, toxins, interferons, ribozymes, anti-sense agents, plasmids, DNA, and RNA. Exemplary growth factors include but are not limited to bone morphogenic proteins (BMP's) and their active fragments or subunits. In some embodiments, the biomolecule is a growth factor, chemotactic factor, cytokine, extracellular matrix molecule, or a fragment or derivative thereof, for example, a cell attachment sequence such as a peptide containing the sequence, RGD.

The term “cells” as used herein has the same meaning as that known in the art. Cell may refer to all types of living or non-living cells from any organism. In certain embodiments, the term cell may also generally refer to a structure that serves as a compartment for other substances. Cell can include stem cells, differentiated cells, or a combination thereof.

The term “composite” as used herein, is used to refer to a unified combination of two or more distinct materials. The composite may be homogeneous or heterogeneous. For example, a composite may be a combination of a polymer and a dispersal agent, a graft and a dispersal agent, or the like. In certain embodiments, the composite has a particular orientation. The term “scaffold” may also be used herein and, depending on the particular usage, is either synonymous with composite or refers solely to a polymer component of a composite. The terms “composite”, “scaffold”, and “graft” may be used interchangeably herein to refer to embodiments of the presently-disclosed subject matter.

The term “contacting” refers to any method of providing or delivering a composite, composition, or the like on to or near tissue to be treated. Such methods are described throughout this document, and include injection of a biodegradable polyurethane scaffold on to a tissue wound and/or molding a biodegradable scaffold in a mold and then placing the molded scaffold on a tissue wound. In some embodiments contacting refers to completely covering a skin wound, and optionally the surrounding skin, with a composite or composition. In some embodiments contacting refers to placing a composite or composition between two or more bone fragments that have fractured. In various aspects, a composite or composition can be contact an existing tissue wound, and in further various aspects they can be contacted prophylactically; that is, to prevent a wound from forming on tissue.

The term “effective amount”, as used herein, refers to an amount of the biodegradable composite sufficient to produce a measurable biological response (e.g., tissue regeneration/repair). Actual dosage levels of the biodegradable composite can be varied so as to administer an amount of antioxidant molecules that is effective to achieve the desired response for a particular subject and/or application. The selected dosage level will depend upon a variety of factors including the type of tissue being addressed, the types of cells and gel beads used, combination with other drugs or treatments, severity of the condition being treated, and the physical condition and prior medical history of the subject being treated. Preferably, a minimal dose is administered, and dose is escalated in the absence of dose-limiting toxicity to a minimally effective amount.

The term “demineralized” is used herein to refer to bone (e.g., particles) that have been subjected to a process that causes a decrease in the original mineral content. As utilized herein, the phrase “superficially demineralized” as applied to bone particles refers to bone particles possessing at least about 90% by weight of their original inorganic mineral content. The phrase “partially demineralized” as applied to the bone particles refers to bone particles possessing from about 8% to about 90% by weight of their original inorganic mineral content, and the phrase “fully demineralized” as applied to the bone particles refers to bone particles possessing less than about 8% by weight, for example, less than about 1% by weight, of their original inorganic mineral content. The unmodified term “demineralized” as applied to the bone particles is intended to cover any one or combination of the foregoing types of demineralized bone particles.

The term “deorganified” as herein applied to matrices, particles, etc., refers to bone or cartilage matrices, particles, etc., that were subjected to a process that removes at least part of their original organic content. In some embodiments, at least 1%, 10%, 20%, 30%, 40%, 50%, 60%, 70%, 80%, 90%, or 99% of the organic content of the starting material is removed. Deorganified bone from which substantially all the organic components have been removed is termed “anorganic.”

The term “flowable polymer material” as used herein, refers to a flowable composition including one or more of monomers, pre-polymers, oligomers, low molecular weight polymers, uncross-linked polymers, partially cross-linked polymers, partially polymerized polymers, polymers, or combinations thereof that have been rendered formable. One skilled in the art will recognize that a flowable polymer material need not be a polymer but may be polymerizable. In some embodiments, flowable polymer materials include polymers that have been heated past their glass transition or melting point. Alternatively or in addition, a flowable polymer material may include partially polymerized polymer, telechelic polymer, or prepolymer. A pre-polymer is a low molecular weight oligomer typically produced through step growth polymerization. The pre-polymer is formed with an excess of one of the components to produce molecules that are all terminated with the same group. For example, a diol and an excess of a diisocyanate may be polymerized to produce isocyanate terminated prepolymer that may be combined with a diol to form a polyurethane. Alternatively or in addition, a flowable polymer material may be a polymer material/solvent mixture that sets when the solvent is removed.

The term “foreign body effect” as used herein, refers to the increased susceptibility to and morbidity of infection that can occur in the presence of an intracorpeal foreign body because its inanimate surfaces are the environmentally eminent domain not of tissue or host defense cells, but rather of microbes. Implanted bone scaffolds may act as a foreign body due to its avascularity. For many orthopaedic implants (e.g., screw, plate, or rod), tissue integration typically wins the race since host tissue cells arrive to the implant first and form a cohesive bond. Consequently, bacteria may be confronted by host immune cells and be less likely to colonize and form a biofilm. Infections centered on biomaterials or bone scaffolds may be difficult to eliminate and usually require removal of the device, which underscores the importance of rapid tissue integration.

The term “mineralized” as used herein, refers to bone that has been subjected to a process that caused a decrease in their original organic content (e.g., de-fatting, de-greasing). Such a process can result in an increase in the relative inorganic mineral content of the bone. Mineralization may also refer to the mineralization of a matrix such as extracellular matrix or demineralized bone matrix. The mineralization process may take place either in vivo or in vitro.

The term “non-demineralized” as herein applied to bone or bone particles, refers to bone or bone-derived material (e.g., particles) that have not been subjected to a demineralization process (i.e., a procedure that totally or partially removes the original inorganic content of bone).

The term “nontoxic” is used herein to refer to substances which, upon ingestion, inhalation, or absorption through the skin by a human or animal, do not cause, either acutely or chronically, damage to living tissue, impairment of the central nervous system, severe illness or death.

The term “osteoconductive” as used herein, refers to the ability of a substance or material to provide surfaces for osteoblast cells to adhere, proliferate, and/or synthesize new bone. Osteoconductive materials include (but are not limited to): cortical-cancellous bone chips (“CCC”); hydroxyapatite (“HA”); tricalcium phosphate (“TCP”); bioactive glass such as Bioglass 45S5; mixtures of at least two of HA, TCP, and bioactive glass (e.g., MasterGraft®, 15% hydroxyapatite and 85% betatricalcium phosphate; BioHorizons, Birmingham, Ala.); other calcium phosphates; calcium carbonate; calcium sulfate; collagen; DBM; other allograft material; and other synthetic allografts. A gathering of one or more types of osteoconductive materials can form an “osteoconductive matrix.” Furthermore, some osteoconductive matrix materials and particles can be referred to as “synthetic allograft” and the like.

The term “osteogenic” as used herein, refers to the ability of a substance or material that can induce bone formation.

The term “osteoinductive” as used herein, refers to the quality of being able to recruit cells (e.g., osteoblasts) from the host that have the potential to stimulate new bone formation and induce ectopic bone formation. In general, osteoinductive materials are capable of inducing heterotopic ossification, that is, bone formation in extra skeletal soft tissues (e.g., muscle).

The term “osteoimplant” is used herein in its broadest sense and is not intended to be limited to any particular shapes, sizes, configurations, compositions, or applications. Osteoimplant refers to any device or material for implantation that aids or augments bone formation or healing. Osteoimplants are often applied at a bone defect site, e.g., one resulting from injury, defect brought about during the course of surgery, infection, malignancy, inflammation, or developmental malformation. Osteoimplants can be used in a variety of orthopedic, neurosurgical, dental, and oral and maxillofacial surgical procedures such as the repair of simple and compound fractures and non-unions, external, and internal fixations, joint reconstructions such as arthrodesis, general arthroplasty, deficit filling, disectomy, laminectomy, anterior cerival and thoracic operations, spinal fusions, etc.

The term “osteotherapeutic material” is used herein to refer to a material that promotes bone growth, including, but are not limited to, osteoinductive, osteoconductive, osteogenic and osteopromotive materials. Further, osteotherapeutic materials, or factors, include: bone morphogenic protein (“BMP”) such as BMP 2, BMP 4, and BMP 7 (OP1); demineralized bone matrix (“DBM”), platelet-derived growth factor (“PDGF”); insulin-like growth factors I and II; fibroblast growth factors (“FGF's”); transforming growth factor beta (“TGF-beta.”); platelet rich plasma (PRP); vescular endothelial growth factor (VEGF); growth hormones; small peptides; genes; stem cells, autologous bone, allogenic bone, bone marrow, biopolymers and bioceramics.

The terms “polynucleotide”, “nucleic acid”, or “oligonucleotide” as used herein, refer to a polymer of nucleotides. The terms “polynucleotide”, “nucleic acid”, and “oligonucleotide”, may be used interchangeably. Typically, a polynucleotide comprises at least three nucleotides. DNAs and RNAs are exemplary polynucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thithymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyriboses, arabinose, and hexose), or modified phosphate groups (e.g., phosphorothioates and 5′-N-phosphoramidite linkages). The polymer may also be a short strand of nucleic acids such as RNAi, siRNA, or shRNA.

The terms “polypeptide”, “peptide”, or “protein” as used herein, include a string of at least three amino acids linked together by peptide bonds. The terms “polypeptide”, “peptide”, and “protein”, may be used interchangeably. In some embodiments, peptides may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc. In one embodiment, the modifications of the peptide lead to a more stable peptide (e.g., greater half-life in vivo). These modifications may include cyclization of the peptide, the incorporation of D-amino acids, etc. None of the modifications should substantially interfere with the desired biological activity of the peptide.

The terms “polysaccharide” or “oligosaccharide” as used herein, refer to any polymer or oligomer of carbohydrate residues. Polymers or oligomers may consist of anywhere from two to hundreds to thousands of sugar units or more. “Oligosaccharide” generally refers to a relatively low molecular weight polymer, while “polysaccharide” typically refers to a higher molecular weight polymer. Polysaccharides may be purified from natural sources such as human, animal (e.g., hyaluronic acid), or other species (e.g., chitosan) and plants (e.g., alginate) or may be synthesized de novo in the laboratory. Polysaccharides isolated from natural sources may be modified chemically to change their chemical or physical properties (e.g., reduced, oxidized, phosphorylated, cross-linked). Carbohydrate polymers or oligomers may include natural sugars (e.g., glucose, fructose, galactose, sucrose, mannose, arabinose, ribose, xylose, etc.) and/or modified sugars (e.g., 2′-fluororibose, 2′deoxyribose, etc.). Polysaccharides may also be either straight or branched. They may contain both natural and/or unnatural carbohydrate residues. The linkage between the residues may be the typical ether linkage found in nature or may be a linkage only available to synthetic chemists. Examples of polysaccharides include cellulose, maltin, maltose, starch, modified starch, dextran, poly(dextrose), and fructose. In some embodiments, glycosaminoglycans are considered polysaccharides. Sugar alcohol, as used herein, refers to any polyol such as sorbitol, mannitol, xylitol, galactitol, erythritol, inositol, ribitol, dulcitol, adonitol, arabitol, dithioerythritol, dithiothreitol, glycerol, isomalt, and hydrogenated starch hydrolysates.

The term “porogen” as used herein, refers to a chemical compound that may be part of the inventive composite and upon implantation/injection or prior to implantation/injection diffuses, dissolves, and/or degrades to leave a pore in the osteoimplant composite. A porogen may be introduced into the composite during manufacture, during preparation of the composite (e.g., in the operating room), or after implantation/injection. A porogen essentially reserves space in the composite while the composite is being molded but once the composite is implanted the porogen diffuses, dissolves, or degrades, thereby inducing porosity into the composite. In this way porogens provide latent pores. In certain embodiments, the porogen may be leached out of the composite before implantation/injection. This resulting porosity of the implant generated during manufacture or after implantation/injection (i. e., “latent porosity”) is thought to allow infiltration by cells, tissue formation, tissue remodeling, osteoinduction, osteoconduction, and/or faster degradation of the osteoimplant. A porogen may be a gas (e.g., carbon dioxide, nitrogen, or other inert gas), liquid (e.g., water, biological fluid), or solid. Porogens are typically water soluble such as salts, sugars (e.g., sugar alcohols), polysaccharides (e.g., dextran (poly(dextrose)), water soluble small molecules, etc. Porogens can also be natural or synthetic polymers, oligomers, or monomers that are water soluble or degrade quickly under physiological conditions. Exemplary polymers include polyethylene glycol, poly(vinylpyrollidone), pullulan, poly(glycolide), poly(lactide), poly(lactide-co-glycolide), other polyesters, and starches. In certain embodiments, tissue and/or sub components or a synthetic analog excipient utilized in provided composites or compositions may act as porogens.

Some embodiments, porogens may refer to a blowing agent (i.e., an agent that participates in a chemical reaction to generate a gas). Water may act as such a blowing agent or porogen.

The term “porosity” as used herein, refers to the average amount of non-solid space contained in a material (e.g., a composite of the present invention). Such space is considered void of volume even if it contains a substance that is liquid at ambient or physiological temperature, e.g., 0.5° C. to 50° C. Porosity or void volume of a composite can be defined as the ratio of the total volume of the pores (i.e., void volume) in the material to the overall volume of composites. In some embodiments, porosity (defined as the volume fraction pores, can be calculated from composite foam density, which can be measured gravimetrically. Porosity may in certain embodiments refer to “latent porosity” wherein pores are only formed upon diffusion, dissolution, or degradation of a material occupying the pores. In such an instance, pores may be formed after implantation/injection. It will be appreciated by these of ordinary skill in the art that the porosity of a provided composite or composition may change over time, in some embodiments, after implantation/injection (e.g., after leaching of a porogen, when osteoclasts resorbing allograft bone, etc.). For the purpose of the present disclosure, implantation/injection may be considered to be “time zero” (T₀). In some embodiments, the present invention provides composites and/or compositions having a porosity of at least about 30%, at least about 40%, at least about 50%, at least about 60%, at least about 70%, at least about 80%, at least about 90% or more than 90%, at time zero. In certain embodiments, pre-molded composites and/or compositions may have a porosity of at least about 30%, at least about 40%, at least about 50%, at least about 60%, at least about 70%, at least about 80%, at least about 90% or more than 90%, at time zero. In certain embodiments, injectable composites and/or compositions may have a porosity of as low as 3% at time zero. In certain embodiments, injectable composites and/or compositions may cure in situ and have a porosity of at least about 30%, at least about 40%, at least about 50%, at least about 60%, at least about 70%, at least about 80%, at least about 90% or more than 90% after curing.

The term “remodeling” as used herein, describes the process by which native tissue, processed tissue allograft, whole tissue sections employed as grafts, and/or other tissues are replaced with new cell-containing host tissue by the action of local mononuclear and multinuclear cells. Remodeling also describes the process by which non-osseous native tissue and tissue grafts are removed and replaced with new, cell-containing tissue in vivo. Remodeling also describes how inorganic materials (e.g., calcium-phosphate materials, such as f3-tricalcium phosphate) is replaced with living tissue.

The term “setting time” as used herein, is approximated by the tack-free time (TFT), which is defined as the time at which the material could be touched with a spatula with no adhesion of the spatula to the foam. At the TFT, the wound could be closed without altering the properties of the material.

The term “subject” as used herein refers to both human and animal subjects. Thus, veterinary therapeutic uses are provided in accordance with the present invention. As such, the present invention provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The term “shaped” as used herein, is intended to characterize a material (e.g., composite) or an osteoimplant refers to a material or osteoimplant of a determined or regular form or configuration in contrast to an indeterminate or vague form or configuration (as in the case of a lump or other solid matrix of special form). Materials may be shaped into any shape, configuration, or size. For example, materials can be shaped as sheets, blocks, plates, disks, cones, pins, screws, tubes, teeth, bones, portions of bones, wedges, cylinders, threaded cylinders, and the like, as well as more complex geometric configurations.

The term “tissue” is used herein to generally refer to an aggregate of cells that perform a particular function or form, at least part of, a particular structure. A particular tissue may comprise one or more types of cells. A non-limiting example of this is skin tissue, bone tissue, tissue of a specific organ, or the like. The term also may refer to certain cell lines. Tissue should not be construed as being limited to any particular organism, but may refer to human, animal, or plant tissue, and may even refer to artificial or synthetic tissue. The term “wound” is used herein to refer to any defect, disorder, damage, or the like of tissue. In some embodiments a wound can be a bone fracture. In some embodiments a wound is damaged skin or skin that must heal from a particular disorder.

The terms “treatment” or “treating” refer to the medical management of a patient with the intent to heal, cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative or prophylactic treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. For example, in some embodiments treatment refers to the healing bone tissue that is fractured and/or healing wounded skin tissue and optionally also eliminating infectious organisms in the area of the treated tissue.

The term “wet compressive strength” as used herein, refers to the compressive strength of a soft tissue implant (STimplant) after being immersed in physiological saline (e.g., phosphate-buffered saline (PBS), water containing NaCl, etc.) for a minimum of 12 hours (e.g., 24 hours). Compressive strength and modulus are well-known measurements of mechanical properties and is measured using the procedure described herein.

The term “working time” as used herein, is defined in the IS0991 7 standard as “the period of time, measured from the start of mixing, during which it is possible to manipulate a dental material without an adverse effect on its properties” (Clarkin et al., J Mater Sci: Mater Med 2009; 20:1563-1570). In some embodiments, the working time for a two-component polyurethane is determined by the gel point, the time at which the crosslink density of the polymer network is sufficiently high that the material gels and no longer flows. According to the present invention, the working time is measured by loading the syringe with the reactive composite and injecting <0.25 ml every 30 s. The working time is noted as the time at which the material was more difficult to inject, indicating a significant change in viscosity.

### BRIEF DESCRIPTION OF THE DRAWINGS

FIG. 1 includes charts showing S. aureus (SA1-4) (A) and P. aeruginosa (PA1-4) (B) biofilm biomass following treatment of pre-formed biofilms with individual D-amino acids at various concentrations. Biofilm dispersal was assess by measuring the absorbance of crystal violet stain at 570 nm following solubilization of the dye in 80% ethanol.

FIG. 2 includes a chart showing bacterial Growth in the Presence of D-AA, where 50 μL of an overnight culture of S. aureus (103-700) and P. aeruginosa (418) were inoculated into 25 mL of MHB-II and grown at 37° C. in the presence or absence of individual D-AA (5 mM; D-Phen, D-Met, D-Trp), and absorbance at was measured every 2 hours up to 12 hours post-inoculation.

FIG. 3 includes charts showing biofilm biomass (OD₅₇₀) following A) treatment of pre-formed biofilms in a panel of clinical strains of S. aureus and P. aeruginosa (n=12) with 5 mM of individual D-AA, and B) co-incubation of the planktonic bacteria with 5 mM of D-AA, and further includes images showing C) CV strained biofilms from S. aureus UAMS-1 and D) P. aeruginosa clinical strain 4 following overnight treatment with individual D-AA.

FIG. 4 includes charts showing biofilm biomass (OD₅₇₀) following treatment of 48 h biofilms of S. aureus UAMS-1 (A) and P. aeruginosa PAO1 (B) with an equimolar mixture of D-Phen, D-Met, and D-Trp for 24 h at 37° C., and further includes C) CLSM images of biofilms of S. aureus UAMS-1 (GFP; top) and P. aeruginosa PAO1 (RFP; bottom) treated with the D-AA mixture for 12, 24, 48 h.

FIG. 5 includes charts showing the viability of human osteoblasts (A) and dermal fibroblasts (B) exposed to media supplemented with D-Phen, D-Met, D-Pro, and D-Trp (50 mM-1 mM) for 24 hours at 37° C. in 5% CO₂.

FIG. 6 includes charts showing bacterial counts (CFU/g) in homogenized bone, hardware, and scaffolds from segmental defects of rats contaminated with 10² CFU (A) or 10⁵ CFU (B) of S. aureus XEN36 or S. aureus UAMS-1 followed by insertion of no scaffold (empty) (n=10), PUR empty scaffold (n=10), or PUR scaffold+D-AA (n=10) for two weeks post wounding, and further includes a chart showing C) a representation of the proportion of contaminated bone, hardware, and scaffolds from the contaminated segmental defects of rats.

FIG. 7 includes charts showing screening of 0.001 mM to 50 mM D-Met, D-Phe, D-Pro, and D-Trp against pre-formed biofilms of four representative clinical isolates of S. aureus, where biofilm dispersal was assessed by quantitating the remaining biofilm biomass following treatment with D-AAs by measuring the absorbance of solubilized CV from the stained biofilms at 570 nm.

FIG. 8 includes charts showing A) dispersion of pre-formed biofilms of four representative clinical isolates of S. aureus with 5 mM of each individual D-AA for 24 h at 37° C., and B) prevention of biofilm formation for the same clinical isolates following co-incubation of the bacteria with 5 mM of D-AA; C) representative images of CV-stained biofilms from S. aureus UAMS-1 following overnight treatment with individual D-AAs; D) a chart showing biofilm biomass (OD₅₇₀) following treatment of pre-formed biofilms of S. aureus UAMS-1 with an equimolar mixture (0.1-5 mM total concentration) of D-Met, D-Pro, and D-Trp for 24 h at 37° C.; and E) images of CV-stained biofilms from S. aureus UAMS-1 following overnight treatment with the mixture of D-AAs (0.1-5 mM).

FIG. 9 includes charts showing the viability of human osteoblasts (A) and dermal fibroblasts (B) exposed to media supplemented with D-Met, D-Phe, D-Pro, and D-Trp (1-50 mM) for 24 hr at 37° C. in 5% CO₂.

FIG. 10 includes A) SEM images showing PUR and PUR+D-AA-10 composites before leaching and after 24 hours leaching, B) a chart showing compressive mechanical properties of dry and wet (soaked in PBS for 24 h) PUR and PUR+D-AA-10 samples, and C) a chart showing cumulative % release of D-Pro, D-Met, and D-Trp versus time.

FIG. 11 includes A) a chart of log₁₀ CFUs/g of UAMS-1 bacteria adhered to PUR composites comprising D-AAs after 24 hour incubation time, and B) SEM images of PUR+D-AA composites exhibiting decreased biofilm with increasing D-AA concentration.

FIG. 12 includes charts showing A) bacterial counts (log₁₀ CFU/g) in homogenized bone from segmental defects of rats contaminated with 10² CFU of S. aureus UAMS-1 followed by implantation of no composite (Empty, n=10), PUR blank composite (PUR, n=10), or PUR composite+equimolar D-AA mixture (n=10) for two weeks post-wounding, and B) the distribution of contaminated and non-contaminated bone samples from the segmental defects.

FIG. 13 includes low and high magnification SEM images of biofilms on PUR and PUR+D-AA-10 composites implanted in contaminated femoral segmental defects in rats for 2 weeks.

FIG. 14 includes images showing PUR/MG (A) and PUR/MG+DAA (B) grafts injected into 11×18 mm femoral condyle defects in sheep, images showing C) a cross-section of a composite explanted at 16 weeks showing host bone (HB), new bone (NB) near the interface, and residual graft (LV) in the inner core, and a chart showing D) BV/TV measured in concentric annular regions (inset) at 16 weeks shows remodeling of the composite progressing from the host bone interface (R=6.4 mm) to the inner core (R=0.9 mm).

FIG. 15 includes charts showing the bacterial radiance for cutaneous wounds treated with blank collagen gels and with collagen gels comprising 5 wt % D-Trp 1 day (A) and 3 days (B) post infection.

FIG. 16 includes charts showing the CFU for cutaneous wounds treated with blank collagen gels and with collagen gels comprising 5 wt % D-Trp 1 day (A) and 3 days (B) post infection.

FIG. 17 includes charts showing the biofilm biomass present for a control biofilm, a biofilm contacted with empty DBM, or a biofilm contacted with DBM comprising a 10% w/w of a 1:1:1 mixture of D-Phe, D-Met, and D-Pro, where the biofilm comprises MSSA bacteria (A) or MRSA bacteria (B).

FIG. 18 includes a chart showing the bacterial attachment, as a function of Log₁₀ CFU/mL per mg, for empty DBM and DBM comprising a 10% w/w of a 1:1:1 mixture of D-Phe, D-Met, and D-Pro exposed to 1 mL of 10⁴ bacteria.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding, and no unnecessary limitations are to be understood therefrom.

The presently-disclosed subject matter includes composites that can comprise a tissue graft and a biofilm dispersal agent. The biofilm dispersal agent can be provided on a surface of a tissue graft, within the tissue graft, or a combination thereof.

The present composites provide novel strategies and methods for treating wounds. Prior systems and methods only focused on the delivery of biofilm dispersal agents from two-dimensional substrates and/or from objects that are not to be implanted or placed on a subject (i.e., not tissue grafts). However, as discussed herein, tissue grafts present a unique set of circumstances because they help treat tissue wounds but can also provide surfaces for the development of biofilms. Furthermore, systematic antibiotic therapy can help reduce the formation of biofilms, but can also bring about several undesirable side effects. Therefore, the present composites can provide a local delivery of biofilm dispersal agents to help reduce or eliminate the formation of biofilms on tissue grafts. The present composites can further comprise other components, such as bioactive agents, that are delivered locally to further enhance the composites' ability to treat wounds and/or prevent the formation of biofilms.

The present inventors have found that tissue grafts can comprise and release biofilm dispersal agents that are effective at dispersing biofilms, thereby naturally reducing bacterial contamination and/or rendering bioactive agents, such as antibiotics, more effective at reducing such contamination. Notably, in some embodiments the composites can comprise and release a quantity of biofilm dispersal agents that is effective at dispersing biofilms while having minimal to no cytotoxic effects. Thus, utilizing composites having biofilm dispersal agents can reduce the quantity or duration for which other bioactive agents need to be administered to prevent or reduce contamination at a wound site. Consequently, the present composites can reduce the time and monetary costs associated with tissue graft treatments, and can also reduce the potential side effects associated with longer term systematic treatments that traditionally are given to treat contamination, including bacterial contamination.

Tissue Graft

The term “tissue graft” as used herein refers to a tissue substitute material, a tissue scaffold, or a combination thereof that can be applied to a tissue wound site that is the result of injury, disease, or surgery. In some embodiments tissue grafts can also be administered to prevent or prophylactically treat a wound. The site of the wound can include a bone site or a soft tissue site. The soft tissue site can include any type of soft tissue, including, but not limited to, a skin tissue site, a vessel tissue site, a nerve tissue site, a tendon tissue site, a ligament tissue site, or a site of any other soft tissue.

The tissue graft material is used to promote healing and tissue regeneration, and can therefore be used to treat a wound. Tissue substitute materials generally replicate the tissue of a subject, and tissue scaffolds provide a surface on which tissue can grow and proliferate. Thus, exemplary tissue grafts include bone grafts, soft tissue grafts, and the like.

Specific examples include allografts, autografts, xenografts, or isografts of bone and/or any soft tissue. Exemplary tissue grafts also include synthetic substitutes for any tissue. For example, the tissue graft may include calcium phosphate, hydroxyapatite, bioactive glass, other osteoconductive materials, and combinations thereof. Tissue grafts may also include bone materials, such as demineralized bone matrix (DBM), mineralized bone, or a combination thereof.

Exemplary tissue grafts can include collagen sponges (collagen gel) or other gels that can be applied to a wound site to help the treatment of the wound. In some embodiments the term tissue graft can be inclusive of medical devices that permit the healing and regeneration of tissue. Still further, some embodiments of tissue grafts include polymeric materials, including biodegradable polymeric materials that can serve as scaffolds for the growth of tissue. In specific embodiments the polymeric material is a polyurethane-based polymeric material.

Polyurethane Polymeric Material

Synthetic polymers can be designed with properties targeted for a given clinical application. According to the present invention, polyurethanes (PUR) are a useful class of biomaterials due to the fact that they can be injectable or moldable as a reactive liquid that subsequently cures to form a porous composite. These materials also have tunable degradation rates, which are shown to be highly dependent on the choice of polyol and isocyanate components (Hafeman et al., Pharmaceutical Research 2008; 25(10):2387-99; Storey et al., J Poly Sci Pt A: Poly Chem 1994; 32:2345-63; Skarja et al., J App Poly Sci 2000; 75:1522-34). Polyurethanes have tunable mechanical properties, which can also be enhanced with the addition of bone particles and/or other components (Adhikari et al., Biomaterials 2008; 29:3762-70; Goma et al., J Biomed Mater Res Pt A 2003; 67A(3):813-27) and exhibit elastomeric rather than brittle mechanical properties.

Polyurethanes can be made by reacting together the components of a two-component composition, one of which includes a polyisocyanate while the other includes a component having two or more hydroxyl groups (i.e., polyols) to react with the polyisocyanate. For example, U.S. Pat. No. 6,306,177, discloses a method for repairing a tissue site using polyurethanes, the content of which is incorporated by reference.

It is to be understood that by “a two-component composition” it means a composition comprising two essential types of polymer components. In some embodiments, such a composition may additionally comprise one or more other optional components.

In some embodiments, polyurethane is a polymer that has been rendered formable through combination of two liquid components (i.e., a polyisocyanate prepolymer and a polyol). In some embodiments, a polyisocyanate prepolymer or a polyol may be a molecule with two or three isocyanate or hydroxyl groups respectively. In some embodiments, a polyisocyanate prepolymer or a polyol may have at least four isocyanate or hydroxyl groups respectively.

Synthesis of porous polyurethane results from a balance of two simultaneous reactions. Reactions, in some embodiments, are illustrated below in Scheme 1. One is a gelling reaction, where an isocyanates and a polyester polyol react to form urethane bonds. The one is a blowing reaction. An isocyanate can react with water to form carbon dioxide gas, which acts as a lowing agent to form pores of polyurethane foam. The relative rates of these reactions determine the scaffold morphology, working time, and setting time.

Exemplary gelling and blowing reactions in forming of polyurethane are shown in Scheme 1 below, where R₁, R₂ and R₃, for example, can be oligomers of caprolactone, lactide and glycolide respectively.

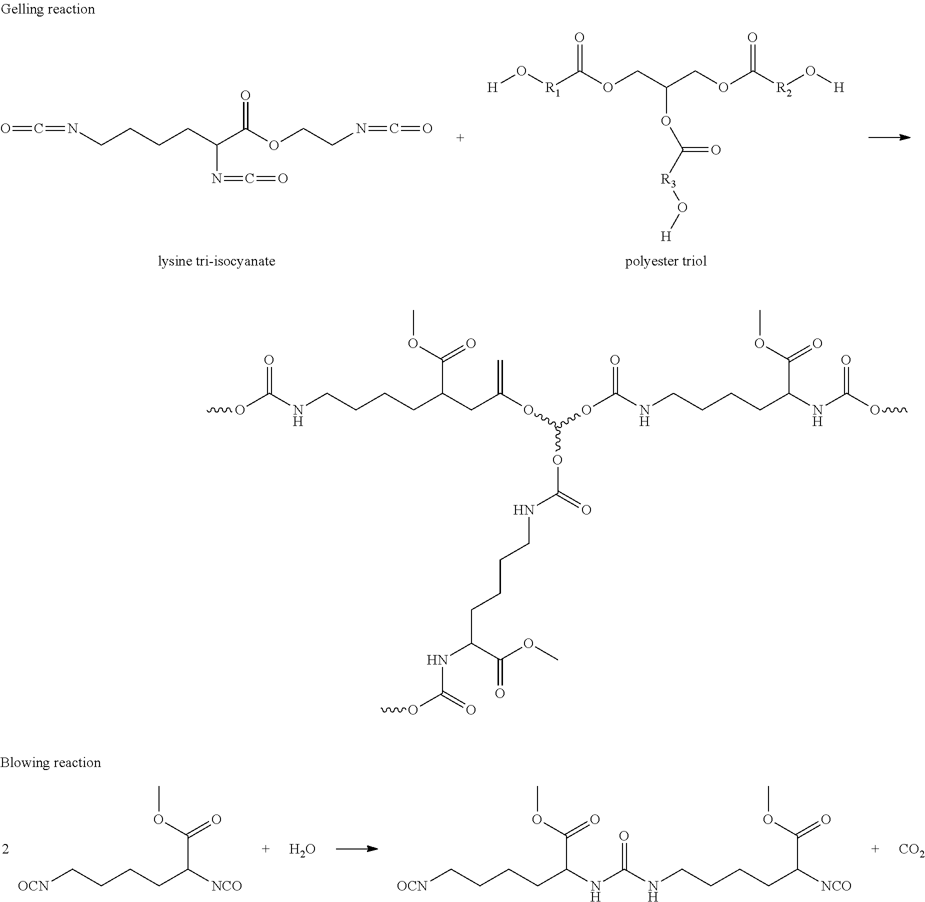

Biodegradable polyurethane scaffolds synthesized from aliphatic polyisocyanates been shown to degrade into non-toxic compounds and support cell attachment and proliferation in vitro. A variety of polyurethane polymers suitable for use in the present invention are known in the art, many of which are listed in commonly owned applications: U.S. Ser. No. 10/759,904 filed on Jan. 16, 2004, entitled “Biodegradable polyurethanes and use thereof” and published under No. 2005-0013793; U.S. Ser. No. 11/667,090 filed on Nov. 5, 2005, entitled “Degradable polyurethane foams” and published under No. 2007-0299151; U.S. Ser. No. 12/298,158 filed on Apr. 24, 2006, entitled “Biodegradable polyurethanes” and published under No. 2009-0221784; all of which are incorporated herein by reference. Polyurethanes described in U.S. Ser. No. 11/336,127 filed on Jan. 19, 2006 and published under No. 2006-0216323, which is entitled “Polyurethanes for Osteoimplants” and incorporated herein by reference, may be used in some embodiments of the present invention. U.S. Ser. No. 12/608,850 also describes exemplary polyurethane composites, and is incorporated herein by reference.

Polyurethanes foams may be prepared by contacting an isocyanate-terminated prepolymer (component 1, e.g, polyisocyanate prepolymer) with a hardener (component 2) that includes at least a polyol (e.g., a polyester polyol) and water, a catalyst and optionally, a stabilizer, a porogen, PEG, etc. In some embodiments, multiple polyurethanes (e.g., different structures, difference molecular weights) may be used in a composite/composition of the present invention. In some embodiments, other biocompatible and/or biodegradable polymers may be used with polyurethanes in accordance with the present invention. In some embodiments, biocompatible co-polymers and/or polymer blends of any combination thereof may be exploited.

Polyurethanes used in accordance with the present invention can be adjusted to produce polymers having various physiochemical properties and morphologies including, for example, flexible foams, rigid foams, elastomers, coatings, adhesives, and sealants. The properties of polyurethanes are controlled by choice of the raw materials and their relative concentrations. For example, thermoplastic elastomers are characterized by a low degree of cross-linking and are typically segmented polymers, consisting of alternating hard (diisocyanates and chain extenders) and soft (polyols) segments. Thermoplastic elastomers are formed from the reaction of diisocyanates with long-chain diols and short-chain diol or diamine chain extenders. In some embodiments, pores in bone/polyurethanes composites in the present invention are interconnected and have a diameter ranging from approximately 50 to approximately 1000 microns.

Prepolymer.

Polyurethane prepolymers can be prepared by contacting a polyol with an excess (typically a large excess) of a polyisocyanate. The resulting prepolymer intermediate includes an adduct of polyisocyanates and polyols solubilized in an excess of polyisocyanates. Prepolymer can, in some embodiments, be formed by using an approximately stoichiometric amount of polyisocyanates in forming a prepolymer and subsequently adding additional polyisocyanates. The prepolymer therefore exhibits both low viscosity, which facilitates processing, and improved miscibility as a result of the polyisocyanate-polyol adduct. Polyurethane networks can, for example, then be prepared by reactive liquid molding, wherein the prepolymer is contacted with a polyester polyol to form a reactive liquid mixture (i.e., a two-component composition) which is then cast into a mold and cured.

Polyisocyanates or multi-isocyanate compounds for use in the present invention include aliphatic polyisocyanates. Exemplary aliphatic polyisocyanates include, but are not limited to, lysine diisocyanate, an alkyl ester of lysine diisocyanate (for example, the methyl ester or the ethyl ester), lysine triisocyanate, hexamethylene diisocyanate, isophorone diisocyanate (IPDI), 4,4′-dicyclohexylmethane diisocyanate (H₁₂MDI), cyclohexyl diisocyanate, 2,2,4-(2,2,4)-trimethylhexamethylene diisocyanate (TMDI), dimers prepared form aliphatic polyisocyanates, trimers prepared from aliphatic polyisocyanates and/or mixtures thereof. In some embodiments, hexamethylene diisocyanate (HDI) trimer sold as Desmodur N3300A may be a polyisocyanate utilized in the present invention. In some embodiments, polyisocyanates used in the present invention includes approximately 10 to 55% NCO by weight (wt % NCO=100*(42/Mw)). In some embodiments, polyisocyanates include approximately 15 to 50% NCO.

Polyisocyanate prepolymers provide an additional degree of control over the structure of biodegradable polyurethanes. Prepared by reacting polyols with isocyanates, NCO-terminated prepolymers are oligomeric intermediates with isocyanate functionality as shown in Scheme 1. To increase reaction rates, urethane catalysts (e.g., tertiary amines) and/or elevated temperatures (60-90° C.) may be used (see, Guelcher, Tissue Engineering: Part B, 14 (1) 2008, pp 3-17).

Polyols used to react with polyisocyanates in preparation of NCO-terminated prepolymers refer to molecules having at least two functional groups to react with isocyanate groups. In some embodiments, polyols have a molecular weight of no more than 1000 g/mol. In some embodiments, polyols have a range of molecular weight between about 100 g/mol to about 500 g/mol. In some embodiments, polyols have a range of molecular weight between about 200 g/mol to about 400 g/mol. In certain embodiments, polyols (e.g., PEG) have a molecular weight of about 200 g/mol. Exemplary polyols include, but are not limited to, PEG, glycerol, pentaerythritol, dipentaerythritol, tripentaerythritol, 1,2,4-butanetriol, trimethylolpropane, 1,2,3-trihydroxyhexane, myo-inositol, ascorbic acid, a saccharide, or sugar alcohols (e.g., mannitol, xylitol, sorbitol etc.). In some embodiments, polyols may comprise multiple chemical entities having reactive hydrogen functional groups (e.g., hydroxy groups, primary amine groups and/or secondary amine groups) to react with the isocyanate functionality of polyisocyanates.

In some embodiments, polyisocyanate prepolymers are resorbable. Zhang and coworkers synthesized biodegradable lysine diisocyanate ethyl ester (LDI)/glucose polyurethane foams proposed for tissue engineering applications. In those studies, NCO-terminated prepolymers were prepared from LDI and glucose. The prepolymers were chain-extended with water to yield biocompatible foams which supported the growth of rabbit bone marrow stromal cells in vitro and were non-immunogenic in vivo. (see Zhang, et al., Biomaterials 21: 1247-1258 (2000), and Zhang, et al., Tiss. Eng., 8(5): 771-785 (2002), both of which are incorporated herein by reference).

In some embodiments, prepared polyisocyanate prepolymer can be a flowable liquid at processing conditions. In general, the processing temperature is no greater than 60° C. In some embodiments, the processing temperature is ambient temperature (25° C.).

In some embodiments the ratio of polyisocyanate to polyol can be adjusted to modify different characteristics of the prepolymer, including its reactivity, viscosity, or the like. In this regard, some embodiments of prepolymers comprise a 2:1 molar ratio of polyisocyanate to polyol. In other embodiments the molar ratio of polyisocyanate to polyol is about 1.5:1, about 1.6:1, about 1.7:1, about 1.8:1, about 1.9:1, about 2.0:1, about 2.1:1, about 2.2:1, about 2.3:1, about 2.4:1, about 2.5:1, about 2.6:1, about 2.7:1, about 2.8:1, about 2.9:1, or about 3.0:1.

In this regard, the viscosity of the prepolymer can also vary depending on different factors. In some embodiments the viscosity of the prepolymer will vary depending on the molar ratio of polyisocyanate to polyol that is used. The viscosity can be tuned so that the composite has desirable workable characteristics (e.g., injectable, putty, etc.), among other things. In some embodiments the viscosity of the prepolymer can be about 10,000 cSt, about 11,000 cSt, about 12,000 cSt, about 13,000 cSt, about 14,000 cSt, about 15,000 cSt, about 16,000 cSt, about 17,000 cSt, about 18,000 cSt, about 19,000 cSt, about 20,000 cSt, about 21,000 cSt, about 22,000 cSt, about 23,000 cSt, about 24,000 cSt, about 25,000 cSt, about 26,000 cSt, about 27,000 cSt, about 28,000 cSt, about 29,000 cSt, or about 30,000 cSt.

Polyols.

Polyols utilized in accordance with the present invention can be amine- and/or hydroxyl-terminated compounds and include, but are not limited to, polyether polyols (such as polyethylene glycol (PEG or PEO), polytetramethylene etherglycol (PTMEG), polypropylene oxide glycol (PPO)); amine-terminated polyethers; polyester polyols (such as polybutylene adipate, caprolactone polyesters, castor oil); and polycarbonates (such as poly(1,6-hexanediol) carbonate). In some embodiments, polyols may be (1) molecules having multiple hydroxyl or amine functionality, such as glucose, polysaccharides, and castor oil; and (2) molecules (such as fatty acids, triglycerides, and phospholipids) that have been hydroxylated by known chemical synthesis techniques to yield polyols.

Polyols used in the present invention may be polyester polyols. In some embodiments, polyester polyols may include polyalkylene glycol esters or polyesters prepared from cyclic esters. In some embodiments, polyester polyols may include poly(ethylene adipate), poly(ethylene glutarate), poly(ethylene azelate), poly(trimethylene glutarate), poly(pentamethylene glutarate), poly(diethylene glutarate), poly(diethylene adipate), poly(triethylene adipate), poly(1,2-propylene adipate), mixtures thereof, and/or copolymers thereof. In some embodiments, polyester polyols can include, polyesters prepared from caprolactone, glycolide, D, L-lactide, mixtures thereof, and/or copolymers thereof. In some embodiments, polyester polyols can, for example, include polyesters prepared from castor-oil. When polyurethanes degrade, their degradation products can be the polyols from which they were prepared from.

In some embodiments, polyester polyols can be miscible with prepared prepolymers used in reactive liquid mixtures (i.e., two-component composition) of the present invention. In some embodiments, surfactants or other additives may be included in the reactive liquid mixtures to help homogenous mixing.

The glass transition temperature (Tg) of polyester polyols used in the reactive liquids to form polyurethanes can be less than 60° C., less than 37° C. (approximately human body temperature) or even less than 25° C. In addition to affecting flowability at processing conditions, Tg can also affect degradation. In general, a Tg of greater than approximately 37° C. will result in slower degradation within the body, while a Tg below approximately 37° C. will result in faster degradation.

Molecular weight of polyester polyols used in the reactive liquids to form polyurethanes can, for example, be adjusted to control the mechanical properties of polyurethanes utilized in accordance with the present invention. In that regard, using polyester polyols of higher molecular weight results in greater compliance or elasticity. In some embodiments, polyester polyols used in the reactive liquids may have a molecular weight less than approximately 3000 Da. In certain embodiments, the molecular weight may be in the range of approximately 200 to 2500 Da or 300 to 2000 Da. In some embodiments, the molecular weight may be approximately in the range of approximately 450 to 1800 Da or 450 to 1200 Da.

In some embodiments, a polyester polyol comprise poly(caprolactone-co-lactide-co-glycolide), which has a molecular weight in a range of 200 Da to 2500 Da, or 300 Da to 2000 Da.

In some embodiments, polyols may include multiply types of polyols with different structures, molecular weight, properties, etc.

Additional Components.

The composites may be used with other agents and/or catalysts. Zhang et al. have found that water may be an adequate blowing agent for a lysine diisocyanate/PEG/glycerol polyurethane (see Zhang, et al., Tissue Eng. 2003 (6):1143-57) and may also be used to form porous structures in polyurethanes. Other blowing agents include dry ice or other agents that release carbon dioxide or other gases into the composite. Alternatively, or in addition, porogens (see detail discussion below) such as salts may be mixed in with reagents and then dissolved after polymerization to leave behind small voids.

Two-component compositions and/or the prepared composites used in the present invention may include one or more additional components. In some embodiments, inventive compositions and/or composites may include, water, a catalyst (e.g., gelling catalyst, blowing catalyst, etc.), a stabilizer, a plasticizer, a porogen, a chain extender (for making of polyurethanes), a pore opener (such as calcium stearate, to control pore morphology), a wetting or lubricating agent, etc. (See, U.S. Ser. No. 10/759,904 published under No. 2005-0013793, and U.S. Ser. No. 11/625,119 published under No. 2007-0191963; both of which are incorporated herein by reference).

In some embodiments, inventive compositions and/or composites may include and/or be combined with encapsulated cells (e.g., stem cell encapsulated in alginate beads). For example, when composites used in tissue healing, solid fillers including cells can help deliver cells to a particular site with limited cell migration and death.

In certain embodiments, additional biocompatible polymers (e.g., PEG) or co-polymers can be used with compositions and composites in the present invention.

Water may be a blowing agent to generate porous polyurethane-based composites. Porosity of bone/polymer composites increased with increasing water content, and biodegradation rate accelerated with decreasing polyester half-life, thereby yielding a family of materials with tunable properties that are useful in the present invention. See, Guelcher et al., Tissue Engineering, 13(9), 2007, pp 2321-2333, which is incorporated by reference. In some embodiments, an amount of water is about 0.5, 1, 1.5, 2, 3, 4 5, 6, 7, 8, 9, 10 parts per hundred parts (pphp) polyol. In some embodiments, water has an approximate rang of any of such amounts.

In some embodiments, at least one catalyst is added to form reactive liquid mixture (i.e., two-component compositions). A catalyst, for example, can be non-toxic (in a concentration that may remain in the polymer). A catalyst can, for example, be present in two-component compositions in a concentration in the range of approximately 0.5 to 5 parts per hundred parts polyol (pphp) and, for example, in the range of approximately 0.5 to 2, or 2 to 3 pphp. A catalyst can, for example, be an amine compound, an iron compound, or a tin compound. In some embodiments, catalyst may be an organometallic compound or a tertiary amine compound. In some embodiments the catalyst may be stannous octoate (an organobismuth compound), triethylene diamine, bis(dimethylaminoethyl)ether, dimethylethanolamine, dibutyltin dilaurate, and Coscat organometallic catalysts manufactured by Vertullus (a bismuth based catalyst), iron acetylacetonate solution, or any combination thereof.

The amount and type of catalyst can be selected to obtain desired curing properties and to modify the kinetics of the composite's polymerization. For example, in embodiments comprising cells, it can be advantageous to decrease the amount of catalyst or selecting a catalyst that is slower acting so as to minimize damage to cells due to the polymerization process. Some catalysts may also be selected based on whether they exhibits a large initial ramp up in reaction rates, whereby the rate at which heat and other harmful substances are released quickly dissipates following the initial burst.

In some embodiments, a stabilizer is nontoxic (in a concentration remaining in the polyurethane foam) and can include a non-ionic surfactant, an anionic surfactant or combinations thereof. For example, a stabilizer can be a polyethersiloxane, a salt of a fatty sulfonic acid or a salt of a fatty acid. In certain embodiments, a stabilizer is a polyethersiloxane, and the concentration of polyethersiloxane in a reactive liquid mixture can, for example, be in the range of approximately 0.25 to 4 parts per hundred polyol. In some embodiments, polyethersiloxane stabilizer are hydrolyzable. In some embodiments, the stabilizer can be a salt of a fatty sulfonic acid. Concentration of a salt of the fatty sulfonic acid in a reactive liquid mixture can be in the range of approximately 0.5 to 5 parts per hundred polyol. Examples of suitable stabilizers include a sulfated castor oil or sodium ricinoleicsulfonate.

Stabilizers can be added to a reactive liquid mixture of the present invention to, for example, disperse prepolymers, polyols and other additional components, stabilize the rising carbon dioxide bubbles, and/or control pore sizes of inventive composites. Although there has been a great deal of study of stabilizers, the operation of stabilizers during foaming is not completely understood. Without limitation to any mechanism of operation, it is believed that stabilizers preserve the thermodynamically unstable state of a polyurethane foam during the time of rising by surface forces until the foam is hardened. In that regard, foam stabilizers lower the surface tension of the mixture of starting materials and operate as emulsifiers for the system. Stabilizers, catalysts and other polyurethane reaction components are discussed, for example, in Oertel, G\ddot{υ}nter, ed., Polyurethane Handbook, Hanser Gardner Publications, Inc. Cincinnati, Ohio, 99-108 (1994). A specific effect of stabilizers is believed to be the formation of surfactant monolayers at the interface of higher viscosity of bulk phase, thereby increasing the elasticity of surface and stabilizing expanding foam bubbles.

To prepare high-molecular-weight polymers, prepolymers are chain can be extended by adding a short-chain (e.g., <500 g/mol) polyamine or polyol. In certain embodiments, water may act as a chain extender. In some embodiments, addition of chain extenders with a functionality of two (e.g., diols and diamines) yields linear alternating block copolymers.

In some embodiments, inventive compositions and/or composites include one or more plasticizers. Plasticizers are typically compounds added to polymers or plastics to soften them or make them more pliable. According to the present invention, plasticizers soften, make workable, or otherwise improve the handling properties of polymers or composites. Plasticizers also allow inventive composites to be moldable at a lower temperature, thereby avoiding heat induced tissue necrosis during implantation. Plasticizer may evaporate or otherwise diffuse out of the composite over time, thereby allowing composites to harden or set. Without being bound to any theory, plasticizer are thought to work by embedding themselves between the chains of polymers. This forces polymer chains apart and thus lowers the glass transition temperature of polymers. In general, the more plasticizer added, the more flexible the resulting polymers or composites will be.

In some embodiments, plasticizers are based on an ester of a polycarboxylic acid with linear or branched aliphatic alcohols of moderate chain length. For example, some plasticizers are adipate-based. Examples of adipate-based plasticizers include bis(2-ethylhexyl)adipate (DOA), dimethyl adipate (DMAD), monomethyl adipate (MMAD), and dioctyl adipate (DOA). Other plasticizers are based on maleates, sebacates, or citrates such as bibutyl maleate (DBM), diisobutylmaleate (DIBM), dibutyl sebacate (DBS), triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC), and trimethylcitrate (TMC). Other plasticizers are phthalate based. Examples of phthalate-based plasticizers are N-methyl phthalate, bis(2-ethylhexyl) phthalate (DEHP), diisononyl phthalate (DINP), bis(n-butyl)phthalate (DBP), butyl benzyl phthalate (BBzP), diisodecyl phthalate (DOP), diethyl phthalate (DEP), diisobutyl phthalate (DIBP), and di-n-hexyl phthalate. Other suitable plasticizers include liquid polyhydroxy compounds such as glycerol, polyethylene glycol (PEG), triethylene glycol, sorbitol, monacetin, diacetin, and mixtures thereof. Other plasticizers include trimellitates (e.g., trimethyl trimellitate (TMTM), tri-(2-ethylhexyl)trimellitate (TEHTM-MG), tri-(n-octyl,n-decyl)trimellitate (ATM), tri-(heptyl,nonyl)trimellitate (LTM), n-octyl trimellitate (OTM)), benzoates, epoxidized vegetable oils, sulfonamides (e.g., N-ethyl toluene sulfonamide (ETSA), N-(2-hydroxypropyl) benzene sulfonamide (HP BSA), N-(n-butyl) butyl sulfonamide (BBSA-NBBS)), organophosphates (e.g., tricresyl phosphate (TCP), tributyl phosphate (TBP)), glycols/polyethers (e.g., triethylene glycol dihexanoate, tetraethylene glycol diheptanoate), and polymeric plasticizers. Other plasticizers are described in Handbook of Plasticizers (G. Wypych, Ed., ChemTec Publishing, 2004), which is incorporated herein by reference. In certain embodiments, other polymers are added to the composite as plasticizers. In certain particular embodiments, polymers with the same chemical structure as those used in the composite are used but with lower molecular weights to soften the overall composite. In other embodiments, different polymers with lower melting points and/or lower viscosities than those of the polymer component of the composite are used.

In some embodiments, polymers used as plasticizer are poly(ethylene glycol) (PEG). PEG used as a plasticizer is typically a low molecular weight PEG such as those having an average molecular weight of 1000 to 10000 g/mol, for example, from 4000 to 8000 g/mol. In certain embodiments, PEG 4000, PEG 5000, PEG 6000, PEG 7000, PEG 8000 or combinations thereof are used in inventive composites. For example, plasticizer (PEG) is useful in making more moldable composites that include poly(lactide), poly(D,L-lactide), poly(lactide-co-glycolide), poly(D,L-lactide-co-glycolide), or poly(caprolactone). Plasticizer may comprise 1-40% of inventive composites by weight. In some embodiments, the plasticizer is 10-30% by weight. In some embodiments, the plasticizer is approximately 10%, 15%, 20%, 25%, 30% or 40% by weight. In other embodiments, a plasticizer is not used in the composite. For example, in some polycaprolactone-containing composites, a plasticizer is not used.

Porosity of inventive composites may be accomplished using any means known in the art. Exemplary methods of creating porosity in a composite include, but are not limited to, particular leaching processes, gas foaming processing, supercritical carbon dioxide processing, sintering, phase transformation, freeze-drying, cross-linking, molding, porogen melting, polymerization, melt-blowing, and salt fusion (Murphy et al., Tissue Engineering 8(1):43-52, 2002; incorporated herein by reference). For a review, see Karageorgiou et al., Biomaterials 26:5474-5491, 2005; incorporated herein by reference. Porosity may be a feature of inventive composites during manufacture or before implantation, or porosity may only be available after implantation. For example, an implanted composite may include latent pores. These latent pores may arise from including porogens in the composite.

Porogens may be any chemical compound that will reserve a space within the composite while the composite is being molded and will diffuse, dissolve, and/or degrade prior to or after implantation or injection leaving a pore in the composite. Porogens may have the property of not being appreciably changed in shape and/or size during the procedure to make the composite moldable. For example, a porogen should retain its shape during the heating of the composite to make it moldable. Therefore, a porogen does not melt upon heating of the composite to make it moldable. In certain embodiments, a porogen has a melting point greater than about 60° C., greater than about 70° C., greater than about 80° C., greater than about 85° C., or greater than about 90° C.

Porogens may be of any shape or size. A porogen may be spheroidal, cuboidal, rectangular, elongated, tubular, fibrous, disc-shaped, platelet-shaped, polygonal, etc. In certain embodiments, the porogen is granular with a diameter ranging from approximately 100 microns to approximately 800 microns. In certain embodiments, a porogen is elongated, tubular, or fibrous. Such porogens provide increased connectivity of pores of inventive composite and/or also allow for a lesser percentage of the porogen in the composite.

Amount of porogens may vary in inventive composite from 1% to 80% by weight. In certain embodiments, the plasticizer makes up from about 5% to about 80% by weight of the composite. In certain embodiments, a plasticizer makes up from about 10% to about 50% by weight of the composite. Pores in inventive composites are thought to improve the osteoinductivity or osteoconductivity of the composite by providing holes for cells such as osteoblasts, osteoclasts, fibroblasts, cells of the osteoblast lineage, stem cells, etc. Pores provide inventive composites with biological in growth capacity. Pores may also provide for easier degradation of inventive composites as bone is formed and/or remodeled. In some embodiments, a porogen is biocompatible.

A porogen may be a gas, liquid, or solid. Exemplary gases that may act as porogens include carbon dioxide, nitrogen, argon, or air. Exemplary liquids include water, organic solvents, or biological fluids (e.g., blood, lymph, plasma). Gaseous or liquid porogen may diffuse out of the osteoimplant before or after implantation thereby providing pores for biological in-growth. Solid porogens may be crystalline or amorphous. Examples of possible solid porogens include water soluble compounds. Exemplary porogens include carbohydrates (e.g., sorbitol, dextran (poly(dextrose)), sucrose, starch), salts, sugar alcohols, natural polymers, synthetic polymers, and small molecules.

In some embodiments, carbohydrates are used as porogens in inventive composites. A carbohydrate may be a monosaccharide, disaccharide, or polysaccharide. The carbohydrate may be a natural or synthetic carbohydrate. In some embodiments, the carbohydrate is a biocompatible, biodegradable carbohydrate. In certain embodiments, the carbohydrate is a polysaccharide. Exemplary polysaccharides include cellulose, starch, amylose, dextran, poly(dextrose), glycogen, etc.

Small molecules including pharmaceutical agents may also be used as porogens in the inventive composites. Examples of polymers that may be used as plasticizers include poly(vinyl pyrollidone), pullulan, poly(glycolide), poly(lactide), and poly(lactide-co-glycolide). Typically low molecular weight polymers are used as porogens. In certain embodiments, a porogen is poly(vinyl pyrrolidone) or a derivative thereof. Plasticizers that are removed faster than the surrounding composite can also be considered porogens.

Biofilm Dispersal Agent

As discussed herein, exemplary composites further comprise a biofilm dispersal agent. Biofilm dispersal agents include substances that can disrupt established biofilms and/or inhibit biofilm development. In this regard, the extent to which biofilms are inhibited can vary depending on type and amount of biofilm dispersal agent as well as the organism that forms the biofilm. In some embodiments the biofilm dispersal agents can inhibit the development of biofilms by about 5%, 10%, 15%, 20%, 25%, 30%, 35%, 40%, 45%, 50%, 55%, 60%, 65%, 70%, 75%, 80%, 85%, 90%, 95%, or more. In some embodiments the biofilm dispersal agents help reduce or eliminate infections, particularly those caused by organisms that form a biofilm, and may also serve to enhance the effects of other biologically active agents.

A non-limiting list of examples of biofilm dispersal agents includes D-amino acids, polyamines, recombinant DNase, bismuth thiols, fatty acids, cis-2-decenoic acid, tetradecanoic acid, 9-hexadecenoic acid, palmic acid, 9,12-linoleic acid, 9-oleic acid, 10-oleic acid, octadecoic acid, 7,10-oleic acid, 5,8,11,14-arachidonic acid, and 7,10,13-eicosatrienoic acid. With regard to D-amino acid biofilm dispersal agents, the type of D-amino acid is not particularly limited, and can be selected from the group consisting of, for example, D-arginine, D-histidine, D-lysine, D-aspartic acid, D-glutamic acid, D-serine, D-threonine, D-asparagine, D-glutamine, D-cysteine, D-selenocysteine, D-proline, D-alanine, D-valine, D-isoleucine, D-leucine, D-methionine, D-phenylalanine, D-tyrosine, and D-tryptophan. See also U.S. Ser. No. 13/520,753, which is incorporated herein in its entirety by this reference.

In specific embodiments the biofilm dispersal agent includes a combination of two or more biofilm dispersal agents. For example, a biofilm dispersal agent can include at least two of D-phenylalanine, D-methionine, D-tryptophan, and D-proline. In specific embodiments the biofilm dispersal agent comprises D-phenylalanine, D-methionine, and D-tryptophan. In other embodiments the biofilm dispersal agent comprises D-methionine, D-tryptophan, and D-proline. In yet further embodiments the biofilm dispersal agent can comprise D-phenylalanine, D-methionine, and D-proline.

In this regard, the relative proportions of biofilm dispersal agents for embodiments comprising a combination of two or more biofilm dispersal agents can be adjusted according to the type of tissue being treated, the severity of the condition and the wound being treated, the type of contamination present at a wound site, and the like. Thus, combinations of two biofilm dispersal agents may be in any suitable proportion, including proportions of about 1:10 to 10:1 of the biofilm dispersal agents. Likewise, relative ratios of combinations of three or more biofilm dispersal agents can also be varied. In exemplary embodiments the biofilm dispersal agents comprise a combination of three biofilm dispersal agents in a 1:1:1 ratio.

The concentration of biofilm dispersal agent to be included in a composite can be varied depending on the intended use for the composite. The concentration or amount of biofilm dispersal agents in a composite can range from about 0.001 wt % 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, or more. Incorporation of biofilm dispersal agents into a tissue graft can reduce the biological burden (e.g., bacterial infection) at a wound site. Without being bound by theory or mechanism, biofilm dispersal agents are particularly effective compared to known antimicrobials at dispersing or eliminating biofilms. Thus, biofilm dispersal agents can be desirable in composites that are in the presence of organisms that can rapidly form biofilms.

Biofilm dispersal agents can also be contained throughout a tissue graft. For example, if the tissue graft is a polymeric material, the biofilm dispersal agent can be incorporated into a component of the polymeric material and/or into the polymeric material in an uncured state. Then, by mixing the composite and allowing the polymeric material to cure, the biofilm dispersal agent remains within the cured polymeric material that forms the tissue graft.

Biofilm dispersal agents can be provided in the form of a pharmaceutically acceptable salt thereof. The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (-ic and -ous), ferric, ferrous, lithium, magnesium, manganese (-ic and -ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

Biofilm dispersal agents can also be provided in an encapsulated form, wherein the biofilm dispersal agents are at least partially contained within or on a substance. In some embodiments the biofilm dispersal agents are provided in microspheres. The microspheres can be about 1 μm, 10 μm, 20 μm, 30 μm, 40 μm, 50 μm, 60 μm, 70 μm, 80 μm, 90, or 100 μm in diameter. Exemplary microspheres include microspheres comprising PLGA.

As described herein, the biofilm dispersal agents can prevent or inhibit the development of biofilms. For certain contamination, however, it can be preferably to administer an antibiotic for a particular bacteria for an extended period of time, such as for about 1 week to about 8 weeks. Furthermore, in some implementations a tissue graft will remain in contact with a wound site for the period of time that the wound heals, which can also be a period of several weeks. Accordingly, in some embodiments the present composites can release biofilm dispersal agents so that the agents are locally delivered for a period of about 1 day, 5 days, 10 days, 15 days, 20 days, 25 days, 30 days, 35 days, 40 days, 45 days, 50 days, 55 days, or 60 days.

The release characteristics of the biofilm dispersal agent can also be tuned. The release characteristics can be tuned depending on the type of tissue graft, the composition of a polymeric material that comprises the tissue graft, encapsulating the biofilm dispersal agent, applying the biofilm dispersal agent on a surface and/or within a tissue graft, or the like. In some embodiments the release of a biofilm dispersal agent from a composite is characterized by an initial burst release followed by a sustained release for a period days or weeks. In some embodiments an initial burst release of about 20% to about 80% of the biofilm dispersal agent can occur over the first 1, 2, 3, 4, 5, 6, 7, 8, 9, or 10 days after the composite is administered. In some implementations a burst release followed by a sustained release can be preferable for dispersing or preventing certain biofilms.

Other Components

Alternatively or additionally, composites of the present composites may have one or more components to deliver when implanted, including cells, encapsulated cells, biomolecules, small molecules, bioactive agents, etc., to promote bone growth and connective tissue regeneration, and/or to accelerate healing. Examples of materials that can be incorporated include chemotactic factors, angiogenic factors, bone cell inducers and stimulators, including the general class of cytokines such as the TGF superfamily of bone growth factors, the family of bone morphogenic proteins, osteoinductors, and/or bone marrow or bone forming precursor cells, isolated using standard techniques. Sources and amounts of such materials that can be included are known to those skilled in the art.

Biologically active materials, comprising biomolecules, small molecules, and bioactive agents may also be included in inventive composites to, for example, stimulate particular metabolic functions, recruit cells, or reduce inflammation. For example, nucleic acid vectors, including plasmids and viral vectors, that will be introduced into the patient's cells and cause the production of growth factors such as bone morphogenetic proteins may be included in a composite. Biologically active agents include, but are not limited to, antiviral agent, antimicrobial agent, antibiotic agent, amino acid, peptide, protein, glycoprotein, lipoprotein, antibody, steroidal compound, antibiotic, antimycotic, cytokine, vitamin, carbohydrate, lipid, extracellular matrix, extracellular matrix component, chemotherapeutic agent, cytotoxic agent, growth factor, anti-rejection agent, analgesic, anti-inflammatory agent, viral vector, protein synthesis co-factor, hormone, endocrine tissue, synthesizer, enzyme, polymer-cell scaffolding agent with parenchymal cells, angiogenic drug, collagen lattice, antigenic agent, cytoskeletal agent, mesenchymal stem cells, bone digester, antitumor agent, cellular attractant, fibronectin, growth hormone cellular attachment agent, immunosuppressant, nucleic acid, surface active agent, hydroxyapatite, and penetration enhancer. Additional exemplary substances include chemotactic factors, angiogenic factors, analgesics, antibiotics, anti-inflammatory agents, bone morphogenic proteins, and other growth factors that promote cell-directed degradation or remodeling of the polymer phase of the composite and/or development of new tissue (e.g., bone). RNAi or other technologies may also be used to reduce the production of various factors.

In some embodiments, inventive composites include antibiotics. Antibiotics may be bacteriocidial or bacteriostatic. An anti-microbial agent may be included in composites. For example, anti-viral agents, anti-protazoal agents, anti-parasitic agents, etc. may be include in composites. Other suitable biostatic/biocidal agents include antibiotics, povidone, sugars, and mixtures thereof. Exemplary antibiotics include, but not limit to, Amikacin, Gentamicin, Kanamycin, Neomycin, Netilmicin, Streptomycin, Tobramycin, Paromomycin, Geldanamycin, Herbimycin, Loravabef, etc. (See, The Merck Manual of Medical Information-Home Edition, 1999). Other bioactive agents include clindamycin, cefazolin, oxacillin, rifampin, trimethoprim/sulfamethoxazole, vancomycin, ceftazadime, ciprofloxacin, colistin, imipenem, and combinations thereof.

Inventive composites may also be seeded with cells. In some embodiments, a patient's own cells are obtained and used in inventive composites. Certain types of cells (e.g., osteoblasts, fibroblasts, dermal cells, stem cells, cells of the osteoblast lineage, etc.) may be selected for use in the composite. Cells may be harvested from marrow, blood, fat, bone, muscle, connective tissue, skin, or other tissues or organs. In some embodiments, a patient's own cells may be harvested, optionally selected, expanded, and used in the inventive composite. In other embodiments, a patient's cells may be harvested, selected without expansion, and used in the inventive composite. Alternatively, exogenous cells may be employed. Exemplary cells for use with the invention include mesenchymal stem cells and connective tissue cells, including osteoblasts, osteoclasts, fibroblasts, preosteoblasts, and partially differentiated cells of the osteoblast lineage. Cells may be genetically engineered. For example, cells may be engineered to produce a bone morphogenic protein.

In some embodiments, inventive composites may include a composite material comprising a component to deliver. For example, a composite materials can be a biomolecule (e.g., a protein) encapsulated in a polymeric microsphere or nanoparticles. In certain embodiments, BMP-2 encapsulated in PLGA microspheres may be embedded in a bone/polyurethane composite used in accordance with the present invention. Sustained release of BMP-2 can be achieved due to the diffusional barriers presented by both the PLGA and Polyurethane of the inventive composite. Thus, release kinetics of growth factors (e.g., BMP-2) can be tuned by varying size of PLGA microspheres and porosity of polyurethane composite. The release of rhBMP-2 from polyurethane scaffolds is further described in U.S. Ser. No. 13/280,299.

To enhance biodegradation in vivo, composites of the present invention can also include different enzymes. Examples of suitable enzymes or similar reagents are proteases or hydrolases with ester-hydrolyzing capabilities. Such enzymes include, but are not limited to, proteinase K, bromelaine, pronase E, cellulase, dextranase, elastase, plasmin streptokinase, trypsin, chymotrypsin, papain, chymopapain, collagenase, subtilisin, chlostridopeptidase A, ficin, carboxypeptidase A, pectinase, pectinesterase, an oxireductase, an oxidase, or the like. The inclusion of an appropriate amount of such a degradation enhancing agent can be used to regulate implant duration.

Components to deliver may not be covalently bonded to a component of the composite. In some embodiments, components may be selectively distributed on or near the surface of inventive composites using the layering techniques described above. While surface of inventive composite will be mixed somewhat as the composite is manipulated in implant site, thickness of the surface layer will ensure that at least a portion of the surface layer of the composite remains at surface of the implant. Alternatively or in addition, biologically active components may be covalently linked to the bone particles before combination with the polymer. As discussed above, for example, silane coupling agents having amine, carboxyl, hydroxyl, or mercapto groups may be attached to the bone particles through the silane and then to reactive groups on a biomolecule, small molecule, or bioactive agent.

Preparation of Composite

Generally, composites can be prepared by combining a tissue graft and a biofilm dispersal agent in any suitable manner. The manner in which a composite is prepared can vary depending on the type of tissue graft, the bacterial contamination being addressed, the type of biofilm dispersal agent, the desired release characteristics, and the like. In some embodiments the composite comprises a tissue graft that includes the biofilm dispersal agent on a surface thereof. In other embodiments the biofilm dispersal agent is within the tissue graft, or impregnated at least within a portion of the tissue graft. Other embodiments comprise a combination such that biofilm dispersal agents are at a surface and at least within a portion of the interior of a tissue graft.

In this regard, tissue grafts can be coated, sprayed, or impregnated with biofilm dispersal agents. Biofilm dispersal agents can also be covered, contacted, loaded, filled into, washed over, or the like on a tissue graft. For instance, the biofilm dispersal agent can be applied via spin coating, dip coating, by brush, spray coating, or the like. The biofilm dispersal agent can be applied to a tissue graft in a solution that comprises the biofilm dispersal agent, wherein the solution can optionally evaporate after the solution has been applied on or into the tissue graft. In some embodiments the tissue graft is partially or fully submerged in a solution that comprises a biofilm dispersal agent. In other embodiments the biofilm dispersal agent can be directly applied within a tissue graft by injecting or otherwise delivering the biofilm dispersal agent to at least a portion of the interior of the tissue graft. The biofilm dispersal agents can also be applied as a dry composition, wherein the dry composition can include labile powders comprising the biofilm dispersal agent.

Still further, biofilm dispersal agents may be provided as a film or composition that is applied on or in a tissue graft. For example, the biofilm dispersal agent can be combined with an adhesive substance that permits the biofilm dispersal agent to adhere to the surface or interior of a tissue graft. Biofilm dispersal agents may also be processed into films, wherein the films can subsequently be applied on to the surface of a tissue graft.

The extent to which the biofilm dispersal agents remain on a surface or absorb into a tissue graft can depend on the type of tissue graft. For instance, absorbing tissue grafts can absorb a biofilm dispersal agent that is only applied to a surface thereof so that the biofilm dispersal agent is within the tissue graft. On the other hand, impermeable tissue grafts that have a biofilm dispersal agent applied thereon can have the biofilm dispersal agent only on the surface of the tissue graft.

Polymeric Tissue Graft Preparation

If the tissue graft is a polymeric material, such as a polyurethane-based tissue graft, the composites can be prepared by combining biofilm dispersal agents, polymers, and, optionally, any additional components. To form inventive composites, a biofilm dispersal agent may be combined with a reactive liquid (i.e., a two-component composition) thereby forming a naturally injectable or moldable composite or a composite that can be made injectable or moldable. Alternatively, a biofilm dispersal agent may be combined with polyisocyanate prepolymers or polyols first and then combined with other components.

In some embodiments, a biofilm dispersal agent may be combined first with a hardener that includes polyols, water, catalysts and optionally a solvent, a diluent, a stabilizer, a porogen, a plasticizer, etc., and then combined with a polyisocyanate prepolymer. In some embodiments, a hardener (e.g., a polyol, water and a catalyst) may be mixed with a prepolymer, followed by addition of a biofilm dispersal agent. In some embodiments, in order to enhance storage stability of two-component compositions, the two (liquid) component process may be modified to an alternative three (liquid)-component process wherein a catalyst and water may be dissolved in a solution separating from reactive polyols. For example, polyester polyols may be first mixed with a solution of a catalyst and water, followed by addition of a biofilm dispersal agent, and finally addition of NCO-terminated prepolymers.

In some embodiments, additional components or components to be delivered may be combined with a reactive liquid prior to injection. In some embodiments, they may be combined with one of polymer precursors (i.e., prepolymers and polyols) prior to mixing the precursors in forming of a reactive liquid/paste.

Porous composites can be prepared by incorporating a small amount (e.g., <5 wt %) of water which reacts with prepolymers to form carbon dioxide, a biocompatible blowing agent. Resulting reactive liquid/paste may be injectable through a 12-ga syringe needle into molds or targeted site to set in situ. In some embodiments, gel time is great than 3 min, 4 min, 5 min, 6 min, 7 min, or 8 min. In some embodiments, cure time is less than 20 min, 18 min, 16 min, 14 min, 12 min, or 10 min.

In some embodiments, catalysts can be used to assist forming porous composites. In general, the more blowing catalyst used, the high porosity of inventive composites may be achieved. In certain embodiments, surprisingly, surface demineralized bone particles may have a dramatic effect on the porosity. Without being bound to any theory, it is believed that the lower porosities achieved with surface demineralized bone particles in the absence of blowing catalysts result from adsorption of water to the hygroscopic demineralized layer on the surface of bones.

Polymers and particles may be combined by any method known to those skilled in the art. For example, a homogenous mixture of polymers and/or polymer precursors (e.g., prepolymers, polyols, etc.) and a biofilm dispersal agent may be pressed together at ambient or elevated temperatures. At elevated temperatures, a process may also be accomplished without pressure. In some embodiments, polymers or precursors are not held at a temperature of greater than approximately 60° C. for a significant time during mixing to prevent thermal damage to any biological component (e.g., biofilm dispersal agent) of a composite. In some embodiments, temperature is not a concern because the biofilm dispersal agent and polymer precursors used in the present invention have a low reaction exotherm. Alternatively or in addition, biofilm dispersal agents may be mixed or folded into a polymer softened by heat or a solvent. Alternatively, a moldable polymer may be formed into a sheet that is then covered with a layer of a biofilm dispersal agent.

After combination with a biofilm dispersal agent, polymers may be further modified by further cross-linking or polymerization. In some embodiments, the composite hardens in a solvent-free condition. In some embodiments, composites are a polymer/solvent mixture that hardens when a solvent is removed (e.g., when a solvent is allowed to evaporate or diffuse away). Exemplary solvents include but are not limited to alcohols (e.g., methanol, ethanol, propanol, butanol, hexanol, etc.), water, saline, DMF, DMSO, glycerol, and PEG. In certain embodiments, a solvent is a biological fluid such as blood, plasma, serum, marrow, etc. In certain embodiments, an inventive composite is heated above the melting or glass transition temperature of one or more of its components and becomes set after implantation as it cools. In certain embodiments, an inventive composite is set by exposing a composite to a heat source, or irradiating it with microwaves, IR rays, or UV light. A composition may be combined and injection molded, injected, extruded, laminated, sheet formed, foamed, or processed using other techniques known to those skilled in the art. In some embodiments, reaction injection molding methods, in which polymer precursors (e.g., polyisocyanate prepolymer, a polyol) are separately charged into a mold under precisely defined conditions, may be employed. For example, a biofilm dispersal agent may be added to a precursor, or it may be separately charged into a mold and precursor materials added afterwards. Careful control of relative amounts of various components and reaction conditions may be desired to limit the amount of unreacted material in a composite. Post-cure processes known to those skilled in the art may also be employed. A partially polymerized polyurethane precursor may be more completely polymerized or cross-linked after combination with hydroxylated or aminated materials or included materials (e.g., a particulate, any components to deliver, etc.).

In some embodiments, an inventive composite is produced with an injectable composition and then set in situ. For example, cross-link density of a low molecular weight polymer may be increased by exposing it to electromagnetic radiation (e.g., UV light) or an alternative energy source. Alternatively or additionally, a photoactive cross-linking agent, chemical cross-linking agent, additional monomer, or combinations thereof may be mixed into inventive composites. Exposure to UV light after a composition is injected into an implant site will increase one or both of molecular weight and cross-link density, stiffening polymers (i.e., polyurethanes) and thereby a composite. Polymer components of inventive composites used in the present invention may be softened by a solvent, e.g., ethanol. If a biocompatible solvent is used, polyurethanes may be hardened in situ. In some embodiments, as a composite sets, solvent leaving the composite is released into surrounding tissue without causing undesirable side effects such as irritation or an inflammatory response. In some embodiments, compositions utilized in the present invention becomes moldable at an elevated temperature into a pre-determined shape. Composites may become set when composites are implanted and allowed to cool to body temperature (approximately 37° C.).

Desired proportions of biofilm dispersal agents relative to the tissue grafts may depend on factors such as injection sites, shape and size of the particles, how evenly polymer is distributed among particles, desired flowability of composites, desired handling of composites, desired moldability of composites, and mechanical and degradation properties of composites. Such proportions can influence various characteristics of the composite, for example, its mechanical properties, including fatigue strength, the degradation rate, and the rate of biological incorporation. In addition, the cellular response to the composite will vary with such proportions. In some embodiments, the desired proportion of biofilm dispersal agents may be determined not only by the desired biological properties of the injected material but by the desired mechanical properties of the injected material.

Inventive composites of the present invention can exhibit high degrees of porosity over a wide range of effective pore sizes. Thus, composites may have, at once, macroporosity, mesoporosity and microporosity. Macroporosity is characterized by pore diameters greater than about 100 microns. Mesoporosity is characterized by pore diameters between about 100 microns about 10 microns; and microporosity occurs when pores have diameters below about 10 microns. In some embodiments, the composite has a porosity of at least about 30%. For example, in certain embodiments, the composite has a porosity of more than about 50%, more than about 60%, more than about 70%, more than about 80%, or more than about 90%. In some embodiments, inventive composites have a porosity in a range of 30%-40%, 40%-45%, or 45%-50%. Advantages of a porous composite over non-porous composite include, but are not limited to, more extensive cellular and tissue in-growth into the composite, more continuous supply of nutrients, more thorough infiltration of therapeutics, and enhanced revascularization, allowing bone growth and repair to take place more efficiently. Furthermore, in certain embodiments, the porosity of the composite may be used to load the composite with biologically active agents such as drugs, small molecules, cells, peptides, polynucleotides, growth factors, osteogenic factors, etc, for delivery at the implant site. Porosity may also render certain composites of the present invention compressible.

In some embodiments, pores of inventive composite may be over 100 microns wide for the invasion of cells and bony in-growth (Klaitwatter et al., J. Biomed. Mater. Res. Symp. 2:161, 1971; which is incorporated herein by reference). In certain embodiments, the pore size may be in a ranges of approximately 50 microns to approximately 750 microns, for example, of approximately 100 microns to approximately 500 microns.

In some embodiments, compressive strength of dry inventive composites may be in an approximate range of 4-10 MPa, while compressive modulus may be in an approximate range of 150-450 MPa. Compressive strength of the wet composites may be in an approximate range of 4-13 MPa, while compressive modulus may be in an approximate 50-350 MPa.

After implantation, inventive composites are allowed to remain at the site providing the strength desired while at the same time promoting healing, regeneration, and/or repair of tissue. Polyurethane of composites may be degraded or be resorbed as new tissue is formed at the implantation site. Polymer may be resorbed over approximately 1 month to approximately 1 years. Composites may start to be remodeled in as little as a week as the composite is infiltrated with cells or new tissue in-growth. A remodeling process may continue for weeks, months, or years. For example, polyurethanes used in accordance with the present invention may be resorbed within about 4-8 weeks, 2-6 months, or 6-12 months. A degradation rate is defined as the mass loss as a function of time, and it can be measured by immersing the sample in phosphate buffered saline or medium and measuring the sample mass as a function of time.

Use and Application of Composite

As discussed above, other components may be supplied separately, e.g., in a kit, and mixed with the composite prior to administration. A surgeon or other health care professional may also combine components in a kit with autologous tissue derived during surgery or biopsy. For example, a surgeon may want to include autogenous tissue or cells, e.g., bone marrow or bone shavings generated while preparing an implant site, into a composite (see more details in co-owned U.S. Pat. No. 7,291,345 and U.S. Ser. No. 11/625,119 published under No. 2007-0191963; both of which are incorporated herein by reference).

Composites of the present invention may be used in a wide variety of clinical applications. A method of preparing and using composite can include providing a tissue graft, applying a biofilm dispersal agent on a surface and/or within the tissue graft, and optionally applying additional components. In some embodiments, the composite can be pre-molded and implanted into a target site. Injectable or moldable composites can be processed (e.g., mixed, pressed, molded, etc.) by hand or machine. Upon implantation, the pre-molded composite may further cure in situ and provide mechanical strength (i.e., load-bearing). A few examples of potential applications are discussed in more detail below. In some methods a composite can be injected or applied on to a wound site that is on soft tissue.

In some embodiments, composites may be used as a void filler. Bone fractures and defects, which result from trauma, injury, infection, malignancy or developmental malformation can be difficult to heal in certain circumstances. If a defect or gap is larger than a certain critical size, natural bone is unable to bridge or fill the defect or gap. These are several deficiencies that may be associated with the presence of a void in a bone. Bone void may compromise mechanical integrity of bone, making bone potentially susceptible to fracture until void becomes ingrown with native bone. Accordingly, it is of interest to fill such voids with a substance which helps voids to eventually fill with naturally grown bone. Open fractures and defects in practically any bone may be filled with composites according to various embodiments without the need for periosteal flap or other material for retaining a composite in fracture or defect. Even where a composite is not required to bear weight, physiological forces will tend to encourage remodeling of a composite to a shape reminiscent of original tissues. The present composites can also be used to fill voids in soft tissue.

Many orthopedic, periodontal, neurosurgical, oral and maxillofacial surgical procedures require drilling or cutting into bone in order to harvest autologous implants used in procedures or to create openings for the insertion of implants. In either case voids are created in bones. In addition to all the deficiencies associated with bone void mentioned above, surgically created bone voids may provide an opportunity for incubation and proliferation of any infective agents that are introduced during a surgical procedure. Another common side effect of any surgery is ecchymosis in surrounding tissues which results from bleeding of the traumatized tissues. Finally, surgical trauma to bone and surrounding tissues is known to be a significant source of post-operative pain and inflammation. Surgical bone voids are sometimes filled by the surgeon with autologous bone chips that are generated during trimming of bony ends of a graft to accommodate graft placement, thus accelerating healing. However, the volume of these chips is typically not sufficient to completely fill the void. Composites and/or compositions of the present invention, for example composites comprising anti-infective and/or anti-inflammatory agents, may be used to fill surgically created bone voids.

### EXAMPLES

The presently-disclosed subject matter is further illustrated by the following specific but non-limiting examples. Some examples are prophetic. Some of the following examples may include compilations of data that are representative of data gathered at various times during the course of development and experimentation related to the presently-disclosed subject matter.

### Example 1

This Example describes methods for preparing polyurethane (PUR) composite comprising D-amino acids, as well as processes for characterizing the effects of PUR comprising D-amino acids on biofilms.

The clinical strains utilized in this Example were single bacterial isolates collected from patients admitted for treatment at Brooke Army Medical Center/San Antonio Military Medical Center (BAMC/SAMMC; Ft. Sam Houston, Tex.) from 2004-2011, confirmed to be positive for biofilm formation, from the clinical molecular biology laboratory repository (Table 1). Green fluorescent protein (GFP) expressing Staphylococcus aureus stain UAMS-1 and red-fluorescent protein (RFP) expressing Pseudomonas aeruginosa strain PAO1 were also used in this Example. With the exception of S. aureus, which was cultured in tryptic soy broth (TSB), all bacteria were grown in Luria-Bertani broth (LB) at 37° C. with constant aeration. Bacterial cultures were frozen and maintained at −80° C. and sub-cultured on blood agar plates (Remel, Lenexa, Kans.) overnight at 37° C. prior to each experimental assay to limit amount of serial passages.

For S. aureus clinical strains the following antibiotics were used: clindamycin (CLD; 128-0.25 μg/mL), cefazolin (CFZ; 128-0.25 μg/mL), oxacillin (OXA; 64-0.125 μg/mL), rifampin (RIF; 64-0.125 μg/mL), trimethoprim/sulfamethoxazole (T/S; 128/2432-0.25/XXX μg/mL), vancomycin (VANC; 64-0.125 μg/mL). For P. aeruginosa, amikacin (AMK; 256-0.5 μg/mL), ceftazadime (TAZ; 128-0.25 μg/mL), ciprofloxacin (CIP; 64-0.125 μg/mL), colistin (CS; 128-0.25 μg/mL), and imipenem (IMI; 128-0.25 μg/mL) were used. All antibiotics were obtained from Sigma Aldrich (St. Louis, Mo.). D and L isoforms of amino acids, including alanine, isoleucine, leucine, methionine, phenylalanine, proline, tryptophan, tyrosine, and valine were obtained from Sigma Aldrich. D-AA stocks were prepared by solubilizing powders in 0.1M NaOH at concentrations between 200-150 mM. Stocks were then diluted into Cation-adjusted Mueller Hinton (MHB-II) broth and neutralized to pH ˜7.4 prior to use in individual experiments.

Biofilm formation was performed under static conditions in polystyrene 96-well plates (Corning, Inc., Corning, N.Y.). Briefly, overnight bacterial cultures were diluted to an OD₆₀₀ of 0.1 in CA-MHB and 10 μL was added to individual wells filled with 190 μl of media and incubated at 37° C. for 48 h without shaking for biofilm growth. Following incubation, culture media was removed and 200 μL media containing individual D-AA at concentrations of 5 mM-1 nM was added. Plates were incubated for an additional 24 h at 37° C. After treatment, plates were gently washed with 1× phosphate buffered saline (PBS; pH 7.4) to remove non-adherent bacteria. Biofilms (i.e. attached cells) were then stained with 0.1% (w/v) crystal violet for 10 min, washed, followed by solubilization of crystal violet with 33% (v/v) acetic acid for 10 min. Biofilm biomass was determined by measuring the absorbance at 570 nm using a microtiter plate reader. For assays measuring the ability of D-AA to block biofilm formation, cells were grown under biofilm conditions as above in the presence of media containing D-AA. Three independent experiments with a minimum of 4 replicates were performed for each experimental assay. Representative images of the plates of CV stained biofilms following treatment with D-AA, prior to solubilization, were taken using a digital camera.

Overnight cultures of bacterial strains were diluted to a final concentration of 5×10⁵ CFU/ml in MHB-II. The resulting bacterial suspension was aliquoted (100 μL) into polystyrene round-bottom 96-well plates containing 100 μL of a 2× concentration of a previously serially diluted (1:2) antibiotic. Each antimicrobial tested was serially diluted 10 times. Following the addition of bacteria, the microtiter plate was covered and was incubated under stationary conditions at 37° C. in the presence of 1× antibiotics. After ˜18 h, the lid was removed and MIC values were recorded by measuring absorbance at 600 nm. MICs of antibiotics were determined in triplicate according to the Clinical and Laboratory Standards Institute (CLSI) broth microdilution as described by the performance standards for antimicrobial susceptibility testing (M100-S22, January 2012). The lowest concentration of antibiotic for which a similar optical density was observed in the inoculated and the blank wells was recorded as the MIC. For each antibiotic, the MIC was determined in the presence or absence of a mixture of D-AA (D-Phenylalanine, D-Methionine, and D-Tryptophan) at an equimolar concentration of 5 mM.

Biofilm susceptibility to antimicrobial agents in the presence and absence of D-AA was determined using the Calgary Biofilm device. Briefly, biofilms from overnight cultures were established as above on individual pegs of the minimum biofilm eradication concentration (MBEC) plates (Innovotech, Canada) with agitation for 48 h at 37° C. Following incubation, the lid with pegs was removed from the incubation plate, washed by re-suspending pegs into plate with wells containing 200 μl of 1×PBS, then placed into a 96-well challenge plate containing pre-diluted antibiotics with or without an equimolar for an additional 24 h at 37° C. Following challenge plates were washed, and the attached viable cells on PEGs within the biofilm were determined by plating serial dilutions onto blood agar plates (Remel). Corresponding minimal biofilm inhibitory concentration (MBIC) was also determined by measuring the optical density in individual wells of the challenge plate at 600 nm. The lowest concentration of antibiotic for which an optical density of <0.100 was recorded as the MBIC. For each antibiotic, the MBIC was determined in the presence or absence of a mixture of D-AA (D-Phenylalanine, D-Methionine, and D-Tryptophan) at an equimolar concentration of 5 mM. Three independent experiments were performed for each of these assays, with a minimum of 3 replicates.

For confocal scanning laser microscopy (CLSM) analysis, biofilms were grown as described above in 8 chamber slides (Thermo-scientific Nunc, Rochester, N.Y.) for 48 h in MHB-II medium. Biofilms were then exposed to media supplemented with or without an equimolar mixture of D-AA (5 mM) for up to 48 h. Following incubation, cells were washed in PBS, fixed with using a 4% formaldehyde solution. Confocal microscopic images were acquired using an Olympus FluoView 1000 Laser Scanning Confocal Microscope (Olympus America Inc., Melville, N.Y.) under 20× magnification using the argon laser at 488 nm and a HeNe-G laser 546±15 nm to visualize GFP and RFP expressing bacteria, respectively. CLSM z-stack image analysis and processing were performed using Olympus Fluoview software. Representative images and stacks of biofilms were acquired from at least three distinct regions on the flow cell. The thickness of the biofilm was measured by performing z-plane scans from 0 μm to 50 μm above the cover glass surface.

To prepare the PUR composite the following were utilized: ε-caprolactone and stannous octoate (Sigma-Aldrich), glycolide and D,L-lactide (Polysciences, Warrington, Pa.), and a isocyanate prepolymer (29.0% NCO) comprising polyethylene glycol (PEG) end-capped with lysine triisocyanate (LTI) (Medtronic, Inc., Memphis, Tenn.).

To fabricate a polyurethane (PUR) graft, polyester triols with a molecular weight of 900 g mol⁻¹ and a backbone comprising 60 wt % ε-caprolactone, 30% glycolide, and 10% lactide (T6C3G1L300) were synthesized using published techniques. Appropriate amounts of dried glycerol and ε-caprolactone, glycolide, DL-lactide, and stannous octoate (0.1 wt-%) were mixed in a 100-ml flask and heated under an argon atmosphere with mechanical stirring to 140° C. for 24 h. The polyester triol was subsequently washed with hexane, dried, and mixed with 0-10 wt % D-amino acids prior to mixing with the LTI-PEG prepolymer. The stoichiometry was controlled such that the excess isocyanate was 15%. The reactive mixture was injected into molds and cured at 37° C. for 24 h.

To observe the cellular viability of PUR/D-AA composites, human dermal fibroblasts and human osteoblasts (PromoCell) were maintained in Dulbecco's Modified Eagle Medium (DMEM; Invitrogen) supplemented with 10% Fetal Bovine Serum (FBS) and 1× penicillin/streptomycin. Human epidermal keratinocytes (HEK-001; ATCC CRL-2404) were maintained in Keratinocyte-serum free media (GIBCO) supplemented with 5 ng/mL human recombinant EGF and 2 mM L-glutamine. All cell lines were cultured at 37° C. in 5% CO₂. Prior to each assay cells were seeded at 100% confluence in black-clear bottom 96-well plates. After 24 h, cells were exposed to media containing D-AA, 50 mM-1 nM, and incubated for 24-48 h at 37° C. in 5% CO₂. Following treatment, cells were washed, resuspended in 100 μL of sterile saline and viability was determined using the CellTiter-Flour Cell Viability Assay (Promega) following the manufacturer's instructions. Viability assays were performed in triplicate with a minimum of 3 replicates. Viability was reported as the percentage of viable cells relative to untreated controls.

Also, kinetic release assays were performed on the PUR scaffolds by incorporating 10 wt % of a 1:1:1 mixture of D-Tr:D-Pro:D-Met incubated in PBS for up to 8 weeks. The medium was be sampled twice weekly and analyzed for D-AAs by HPLC.

Lastly, the scaffolds were tested in a rat femoral 6 mm segmental defect model. A characterized contaminated critical size defect in rat (Sprague-Dawley) femurs was utilized as the in vivo model of infection. Briefly, a 6-mm segmental defect was created using a small reciprocating saw blade (MicroAire 1025, MicroAire, Charlottesville, Va.), stabilized with a polyacetyl plate (length 25 mm, width 4 mm and height 4 mm) and fixed to the surface of femur using threaded K-wires. The defects in all animals were then implanted with 30 mg of type I bovine collagen (Stryker Biotech, Hopkinton, Mass.) wetted with 10⁵ colony-forming units (CFU) of S. aureus Xenogen 36 (Caliper Life Science, Hopkinton, Mass.) or S. aureus UAMS-1 (University of Arkansas Medical Service). Six hours after contamination, the wounds were opened, debrided, and irrigated with saline. Empty PUR grafts or PUR grafts comprising D-AA were then placed into the wounds. Two weeks following surgery, the femur with defect and associated graft and hardware was harvested from the animals and used for quantitative assessment. Femurs were weighed, snap-frozen in liquid nitrogen, ground to a fine powder, and resuspended in saline. Similarly, rafts and hardware were vortexed and sonicated in saline. CFUs were determined plating serial dilutions onto TSB plates and incubated at 37° C. for 48 h. CFUs were expressed as log₁₀ CFU per gram of tissue/substrate.

For pair-wise comparisons of groups statistical analyses were performed using a Student's t-test. For multivariate analyses a 1-Way ANOVA followed by a post-priori test using Sigma Stat software was used.

### Example 2

This Example demonstrates the results obtained from the procedures described in Example 1. This Example also describes the effects of D-amino acids on biofilms. This Example further describes the incorporation of D-AA into PUR composites and characterizes methods of using the PUR composites for treating wounds.

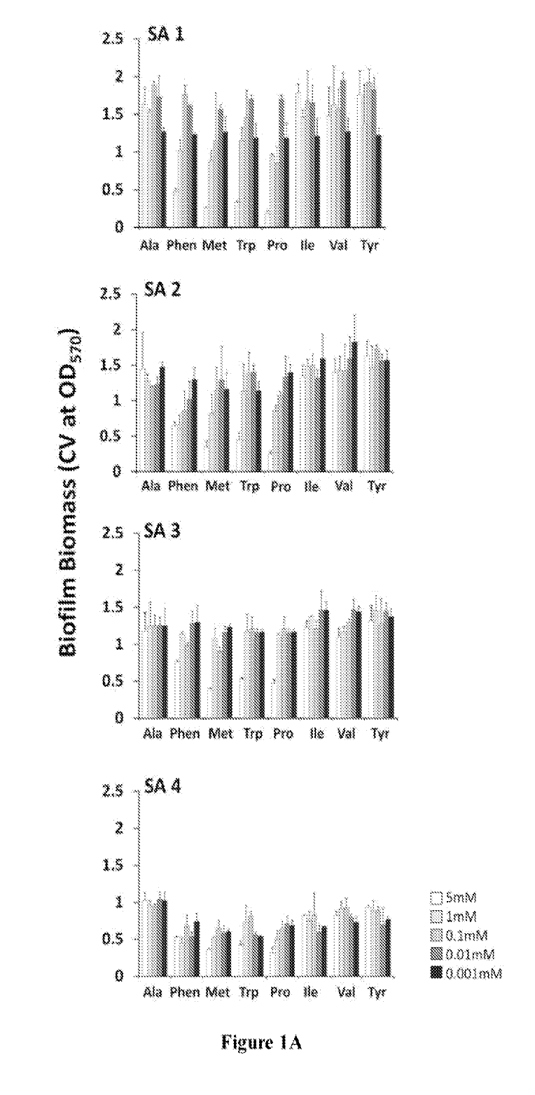

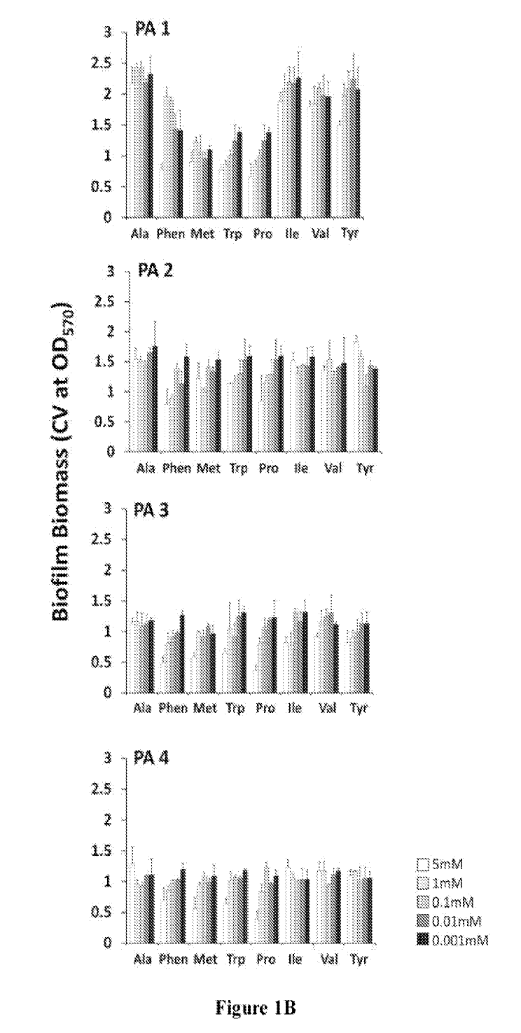

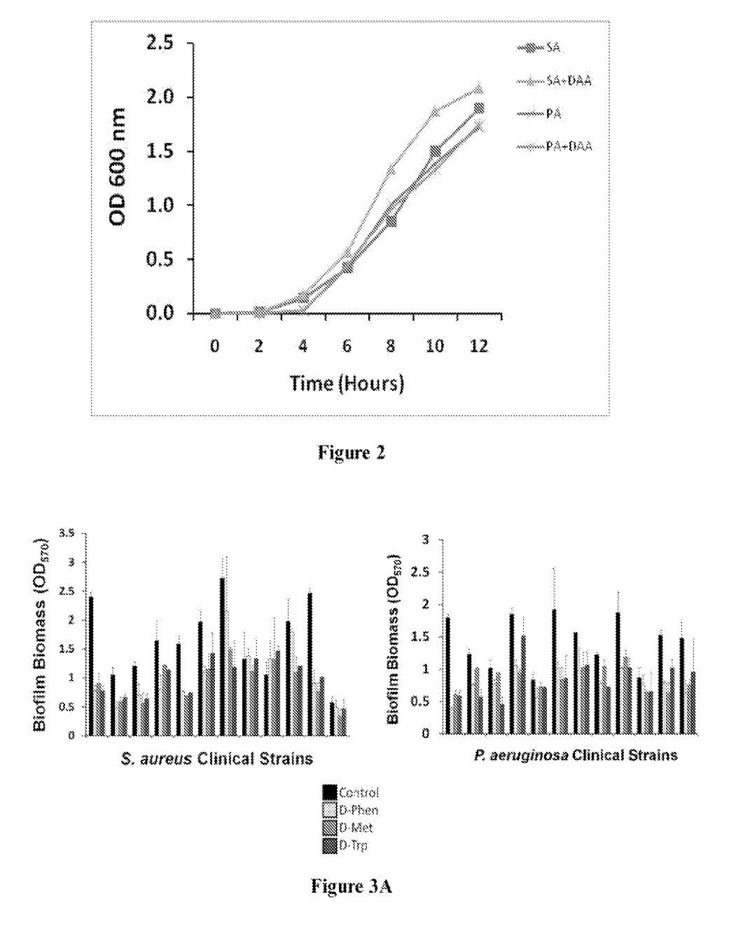

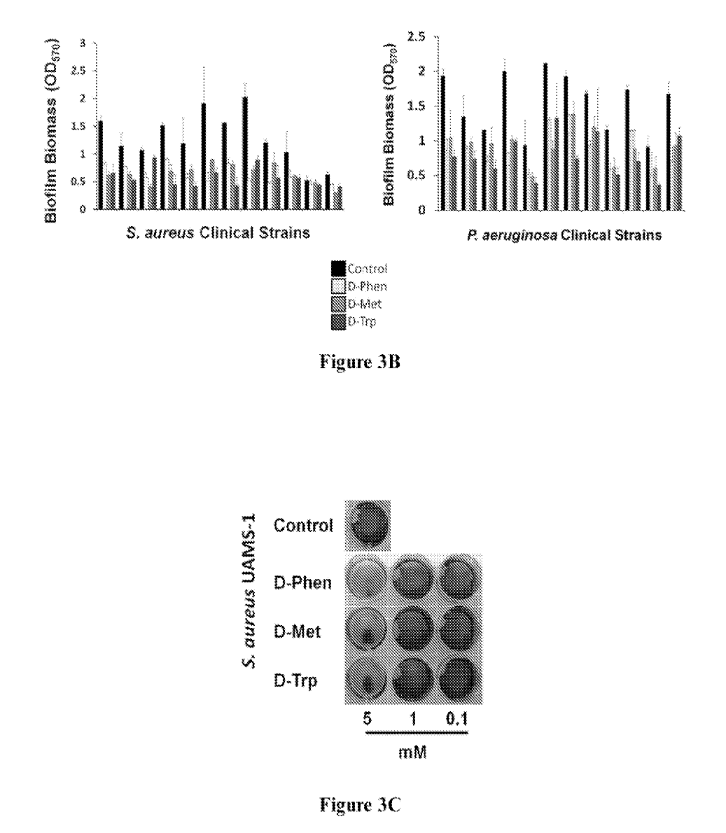

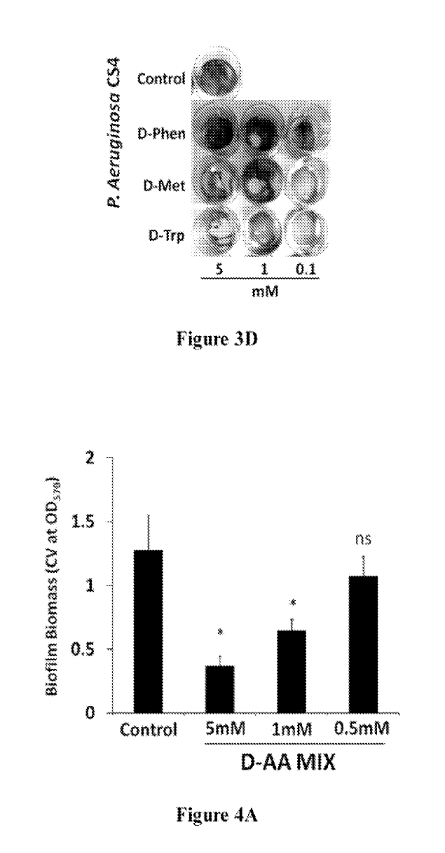

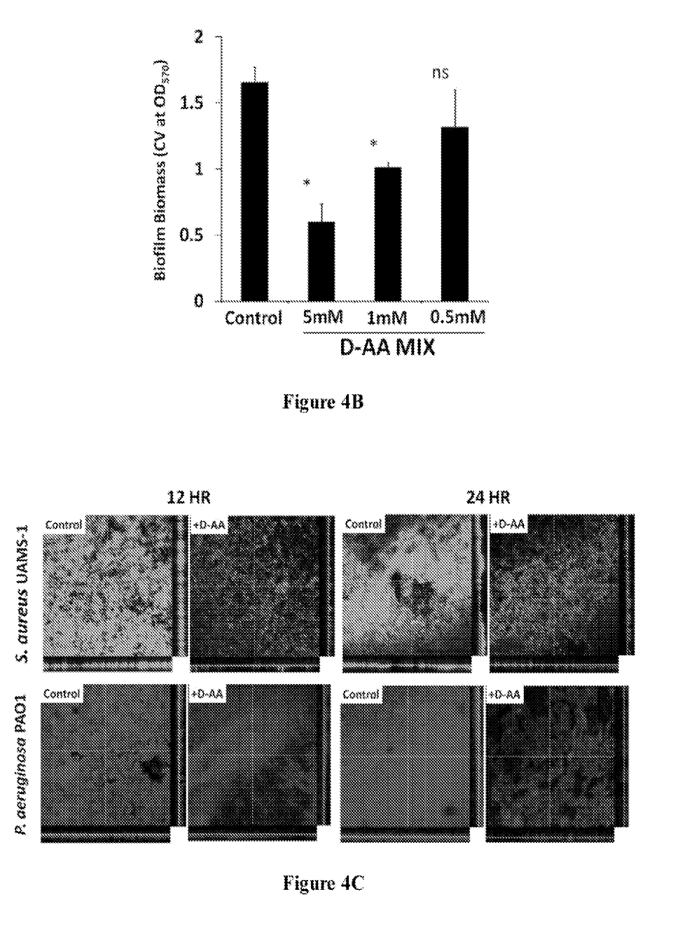

To evaluate the clinical application of D-amino acids as an antibiofilm strategy, D-AA's ability to disperse and prevent biofilm formation in a panel of clinical isolates of S. aureus and P. aeruginosa was first tested. Pre-screening of a panel of eight D-AAs identified four D-AAs, including D-Phen, D-Met, D-Trp, and D-Pro, to be relatively more effective at dispersing biofilms of S. aureus and P. aeruginosa at 5 mM (FIG. 1). The antibiofilm effect was isoforms specific, as no dispersal activity was observed with L-isoforms of D-AAs. When tested against the panel of clinical isolates of S. aureus and P. aeruginosa, D-Phen, D-Met, D-Trp, and D-Pro, were capable of significantly dispersing biofilm formation in vitro as determined by measurement of the biofilm biomass at OD₅₇₀ nm (FIGS. 3A and 3C). The antibiofilm activity of the individual D-AAs was both strain/species dependent, although for each strain tested more than one of the four D-AA was effective at dispersing biofilms. In addition to dispersal, D-AAs were effective in preventing biofilm formation in S. aureus and P. aeruginosa clinical strains (FIG. 3B). When combined into an equimolar mixture of D-Phen, D-Met, and D-Trp, antibiofilm activity was enhanced and effective at concentrations <5 mM (FIG. 4A). Analysis by confocal laser scanning microscopy (CLSM) demonstrated the potency of the D-AA mixture, which reduced biomass as early as 12 h continuing until little or no biofilm was observed at 48 h (FIG. 4B). The D-AAs individually or when combined had no significant effect on the growth of S. aureus and P. aeruginosa cells, indicating that biofilm dispersal was not the result of growth inhibition (FIG. 2).

Furthermore, to determine if the D-AAs could enhance the effect of conventional antimicrobials against biofilms, antimicrobial susceptibility assays with and without the D-AA mixture were performed on both the planktonic and biofilm derived bacteria from two clinical strains of S. aureus (UAMS-1 and 103-700) and P. aeruginosa (418 and 18189). MICs of antimicrobials were determined for planktonic S. aureus strains UAMS-1 and 103-700, CLI (0.5 μL/mL), CFZ (0.5 μL/mL; 16 μL/mL), OXA (1 μg/mL; 8 μg/mL), RIF (0.5 μg/mL), and VAN (0.5 μg/mL; 1 μg/mL). Similarly, the MICs were determined for the P. aeruginosa strains 418 and 18189, AMK (16 μg/mL), CS (1 μg/mL), CIP (4 μg/mL; 8 μg/mL), IMI (128 μg/mL; 64 μg/mL), and TAZ (16 μg/mL; 2 μg/mL).

When used alone, the vast majority of conventional antimicrobials were ineffective at reducing the number of bacteria within biofilms of both S. aureus and P. aeruginosa (Tables 1 and 2). With the exception of rifampin and to some extent colistin, the minimal biofilm inhibitory concentrations (MBIC) were at the higher end or often exceeded the antimicrobial ranges tested. Treatment of the biofilms with antimicrobials in combination with the D-AA mixture resulted in an enhanced effect against bacterial biofilms. Of the antimicrobials tested for S. aureus, the use of the D-AA mixture enhanced the effect of rifampin, clindamycin, and vancomycin resulting in significant reductions of bacterial CFUs within the biofilms of both strains (Tables 2 and 4). Similarly, the D-AA mixture enhanced the effect of colistin, ceftazadime, and somewhat that of ciprofloxacin against biofilms of P. aeruginosa strains 418 and 18189 (Table 3). As above, the use of D-AA with these antimicrobials resulted in decreases in bacteria within biofilms (Table 3).

TABLE 1
Characteristics of Clinical Isolates.

| Bacterial | Clinical | # of | Pulse Field | | |
|---|---|---|---|---|---|
| species | Isolates | Patients | Type (PFTs) | Phenotypea | Site of Isolation |
| P. aeruginosa | 11 | 8 | 1 (n = 2) | MDR (n = 10) | Wound culture (n = 9) |
| | | | 2, 18 (n = 3) | | Blood (n = 3) |
| | | | Other (n = 4) | | |
| S. aureus | 10 | 10 | USA 100 (n = 2) | MRSA (n = 9) | Wound culture (n = 8) |
| | | | USA 200, USA | MSSA (n = 3) | Blood (n = 2) |
| | | | 800 (n = 3) | | |
| | | | USA 300 (n = 2) | | Bone (n = 2) |
| | | | USA 700 (n = 1) | | |

^{a}A multidrug resistant (MDR) organism was defined as any extended spectrum beta-lactamase (ESBL)-producing bacteria, or if resistant to all tested antimicrobials in 3 or more classes of antimicrobial agents (penicillins/cephalosporins, carbapenems, aminoglycosides, and quinolones) not including tetracyclines or colistin.

TABLE 2
MIC and MBIC of different antibiotics alone or in combination with D-amino
acids against planktonic and biofilm derived S. aureus clinical strains.

| Antimicrobial | | Planktonic (MIC) | | | | Biofilm (MBIC)^{a} | | | |
|---|---|---|---|---|---|---|---|---|---|
| Agent^{c} | Class | UAMS-1 | | 103-700 | | UAMS-1 | | 103-700 | |
| CLI | Lincosamide | 0.5 | μg/mL | 0.5 | μg/mL | 64 | μg/mL | >64 | μg/mL |
| CLI* | | 0.25 | μg/mL | 0.5 | μg/mL | 16 | μg/mL | 32 | μg/mL |
| CFZ | Cephem | 0.5 | μg/mL | 16 | μg/mL* | >128 | μg/mL | >128 | μg/mL |
| CFZ* | | 0.5 | μg/mL | 16 | μg/mL* | 128 | μg/mL | >128 | μg/mL |
| OXA | Penicillin | 1 | μg/mL | 8 | μg/mL* | >64 | μg/mL | >64 | μg/mL |
| OXA* | | 1 | μg/mL | 8 | μg/mL* | >64 | μg/mL | >64 | μg/mL |
| RIF | Ansamycin | 0.5 | μg/mL | 0.5 | μg/mL | 32 | μg/mL | 32 | μg/mL |
| RIF* | | 0.5 | μg/mL | 0.5 | μg/mL | 8 | μg/mL | 16 | μg/mL |
| T/S | Folate Pathway | | | | | >128/2432 | μg/mL | >128/2432 | μg/mL |
| T/S* | Inhibitor | | | | | >128/2432 | μg/mL | >128/2432 | μg/mL |
| VAN | Glycopeptide | 0.5 | μg/mL | 1 | μg/mL | >64 | μg/mL | >64 | μg/mL |
| VAN* | | 0.5 | μg/mL | 1 | μg/mL | 32 | μg/mL | 16 | μg/mL |

^{a}Minimal Inhibitory Concentration (MIC) and Minimal Inhibitory Biofilm Concentration (MBIC) of the antibiotic (μg/mL) alone or when used in combination with equimolar amounts (5 mM) of D-Phenylalanine. D-Tryptophan and D-Methionine.
^{b}MIC was determined as the concentration of antibiotic that resulted in >90 reduction in planktonic culture or and OD₆₀₀ >0.1; MBIC is the biofilm equivalent of MIC and corresponds to an OD₆₀₀ >0.1.
^{c}Clindamycin (CLI), Cefazolin (CFZ), Oxacillin (OXA), Rifampin (RIF), Trimethoprim-sulfamethoxazole (T/S), Vancomycin (VAN)

TABLE 3
MIC and MBIC of different antibiotics alone or in combination with D-amino acids
against planktonic and biofilm derived P. aeruginosa clinical strains.

| Antimicrobial | | Planktonic (MIC)^{a} | | | | Biofilm (MBIC)^{a} | | | |
|---|---|---|---|---|---|---|---|---|---|
| Agent^{b} | Class | 18189 | | 418 | | 18189 | | 418 | |
| AMK | Aminoglycoside | 16 | μg/mL | 16 | μg/mL | >256 | μg/mL | >256 | μg/mL |
| AMK* | | 8 | μg/mL | 16 | μg/mL | >256 | μg/mL | >256 | μg/mL |
| CS | Lipopeptide | 1 | μg/mL | 1 | μg/mL | 64 | μg/mL | 128 | μg/mL |
| CS* | | 1 | μg/mL | 1 | μg/mL | 16 | μg/mL | 32 | μg/mL |
| CIP | Fluoroquinolone | 4 | μg/mL* | 8 | μg/mL* | 64 | μg/mL | >64 | μg/mL |
| CIP* | | 4 | μg/mL | 8 | μg/mL | 32 | μg/mL | 64 | μg/mL |
| IMI | Carbapenem | 128 | μg/mL* | 64 | μg/mL* | >128 | μg/mL | >128 | μg/mL |
| IMI* | | 128 | μg/mL | 64 | μg/mL | >128 | μg/mL | 64 | μg/mL |
| TAZ | Cephem | 16 | μg/mL* | 2 | μg/mL | >128 | μg/mL | >128 | μg/mL |
| TAZ* | | 8 | μg/mL | 2 | μg/mL | 64 | μg/mL | 32 | μg/mL |

^{a}Minimal Inhibitory Concentration (MIC) and Minimal Inhibitory Biofilm Concentration (MBIC) of the antibiotic (μg/mL) alone or when used in combination with equimolar amounts (5 mM) of D-Phenylalanine, D-Tryptophan and D-Methionine.
^{b}Amikacin (AMK), Colistin (CS), Ciprofloxacin (CIP), Imipenem (IMI), Ceftazadime (TAZ)

TABLE 4
Percent decrease in CFU of biofilms treated antibiotics
alone or combination with D-amino acids.

| | | % Decrease + SD in CFU^{a} | | | |
|---|---|---|---|---|---|
| Strain | Treatment | 64 μg/mL | 32 μg/mL | 16 μg/mL | 8 μg/mL |
| S. aureus UAMS-1 | CLI | 66.0 ± 3.4 | 32.86 ± 4.3 | 9.3 ± 1.8 | 1.8 ± 1.0 |
| | CLI-DAA | 70.64 ± 4.2 | 71.22 ± 1.2* | 66.30 ± 3.1* | 32.7 ± 2.4* |
| | RIF | 67.36 ± 1.2 | 70.70 ± 4.3 | 34.31 ± 5.4 | 16.87 ± 2.9 |
| | RIF-DAA | 69.99 ± 5.3 | 70.46 ± 2.8 | 69.73 ± 2.1* | 68.54 ± 3.3* |
| | VAN | 65.97 ± 5.3 | 31.90 ± 7.1 | 15.93 ± 6.2 | 1.5 ± 5.7 |
| | VAN-DAA | 68.58 ± 2.8 | 70.05 ± 3.7* | 48.75 ± 4.1* | 32.24 ± 4.8* |
| P. aeruginosa 18189 | CS | 41.98 ± 4.2 | 32.70 ± 2.1 | 19.53 ± 1.8 | 0.96 ± 3.4 |
| | CS-DAA | 55.61 ± 6.1 | 55.53 ± 3.5 | 32.19 ± 4.2 | 22.09 ± 2.3* |
| | CIP | 49.37 ± 3.2 | 38.12 ± 4.2 | 21.54 ± 4.0 | 10.57 ± 2.3 |
| | CIP-DAA | 53.03 ± 5.4 | 48.99 ± 3.8 | 39.87 ± 3.8 | 32.11 ± 3.9* |
| | TAZ | 31.07 ± 4.5 | 31.90 ± 2.2 | 18.20 ± 3.5 | 4.76 ± 3.2 |
| | TAZ-DAA | 61.02 ± 2.0* | 55.94 ± 3.1 | 31.16 ± 2.1* | 22.12 ± 1.4* |

^{a}% of viable bacteria (Log10(CFU/mL) within biofilm following treatment with select antimicrobial agents compared to control non-treated biofilms.

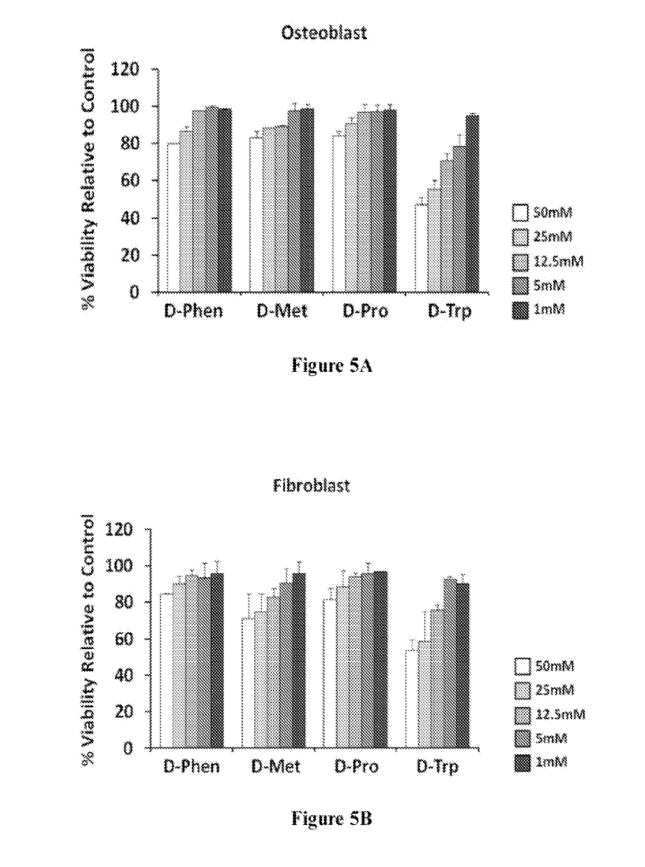

The scaffold that were produced comprising D-amino acids and that were used for in vitro study had a porosity of about 89±1 vol % for 0 wt % D-AA and about 90±2 vol % for 10 wt % D-AA. The pore size varied from about 100 to about 500 μm. Thereafter, to assess whether D-AAs could have a negative effects on wound healing in vivo, the cytotoxicity of individual D-AAs in human osteoblasts and dermal fibroblasts, cell lines relevant to wound healing, were tested. With the exception of D-Tryptophan, exposure to all other D-AAs, at concentrations exceeding effective ranges, had minimal to no observable cytotoxicity in either the osteoblast and fibroblast cell lines exposed for 24 h (FIGS. 5A and 5B). Although, D-Trp at concentrations of 50 and 25 mM was toxic to both cell lines, at concentrations within the effective range, 1-5 mM, minimal cytotoxicity was observed. In human fibroblasts, cytotoxicity was also observed with the higher concentrations of D-Methionine.

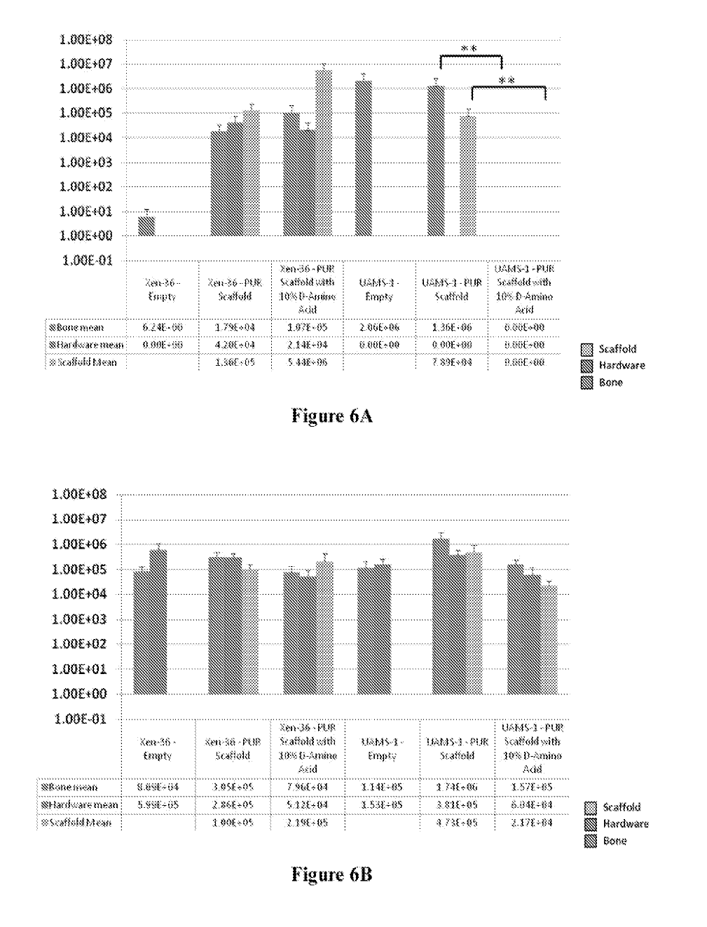

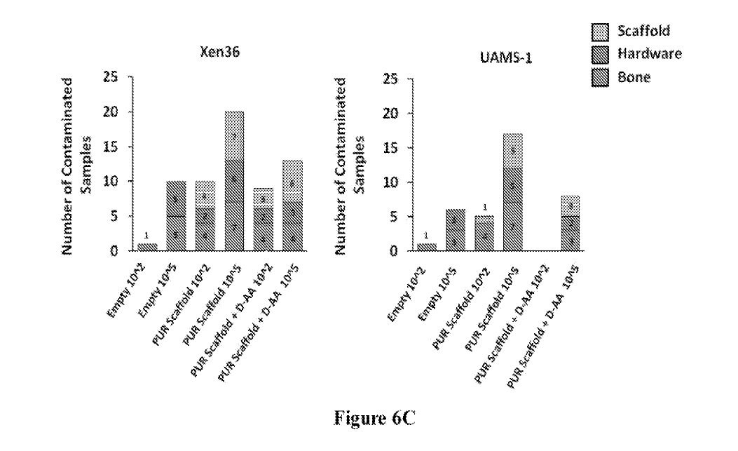

To evaluate whether D-AA could be used in vivo to reduce bacterial burden within wounds, the effects of local delivery of D-AA in PUR scaffolds on bacterial growth were evaluated in a contaminated rat segmental defect model. For these studies rat defects were contaminated with a clinical septicemia strain, S. aureus XEN36, or an osteomyelitis strain, S. aureus UAMS-1, at 10² or 10⁵ CFUs. Incorporation of the D-AAs into the PUR scaffolds minimally reduced the contamination within those defects infected with Xen36 (FIGS. 6A and 6B), whereas D-AAs significantly reduced bacterial burdens in UAMS-1 contaminated defects. Compared to the empty scaffolds, addition of PUR scaffolds containing a 10% w/v equal ratio of D-Phen, D-Met, and D-Pro into femoral defects reduced the bacterial contamination within the homogenized bone and scaffolds >6 logs (p<0.005) at the lower infectious dose (FIG. 6A). Supporting this result, SEM analysis of scaffolds with D-AA had less bacterial contaminants on their surface, and those bacteria present within the scaffolds predominately existed as biofilms (not shown). In contrast, at the higher infectious dose D-AA had only a moderate effect on reducing bacterial contamination within the bone, scaffolds and hardware reducing the bacterial contaminants <1 log in most cases (FIG. 6B). When comparing the proportion of contaminated bone, the results demonstrate that D-AA's can prevent contamination (FIG. 6C).

### Example 3

This Example describes procedures conducted to characterize the ability of D-amino acids to disperse and prevent biofilms of strains of S. aureus.

D-isomers and L-isomers of amino acids (free base form), including alanine, isoleucine, leucine, methionine, phenylalanine, proline, tryptophan, tyrosine, and valine, were obtained (Sigma Aldrich). For use in bacterial and cell cultures, D-AA stocks were prepared by dissolving powders in 0.5 M HCl at concentrations between 150-200 mM. Stocks were then diluted into cation-adjusted Mueller Hinton (MHB-II) broth neutralized to pH 7.4 and stored at −80° C.

Four clinical isolates of S. aureus from a repository collected from patients admitted for treatment not related to research at the San Antonio Military Medical Center (SAMMC, Ft. Sam Houston, Tex.) were used. Characteristics of four clinical isolates, which were previously confirmed to be positive for biofilm formation, are described in Table 5. UAMS-1 (ATCC strain 49230) is a methicillin-susceptible S. aureus strain of the USA200 clonal group. Xen36 is a bioluminescent strain modified with the luxABCDE operon (Caliper Life Sciences Inc., Hopkinton, Mass.) derived from a methicillin-sensitive clinical bacteremia isolate of S. aureus subsp. Wright (ATCC 49525). All bacterial strains were cultured in tryptic soy broth (TSB) with agitation or on blood agar plates overnight at 37° C.

TABLE 5
Description of S. aureus strains investigated.

| | | Biofilm |
|---|---|---|
| Strain | Strain Characteristics | formation¹ |
| UAMS-1 | ATCC strain 49230. Methicillin-susceptible | Strong |
| | strain of the USA200 clonal group and a | |
| | well-characterized osteomyelitis isolate. | |
| Xenogen 36 | Xen36 is a bio luminescent strain modified | Weak |
| | with the luxABCDE operon derived from a | |
| | methicillin-sensitive clinical bacteremia | |
| | isolate of S. aureus subsp. Wright | |
| | (ATCC 49525). | |
| S. aureus | Methicillin-resistant strain of the USA300 | Strong |
| Clinical | clonal group; wound isolate | |
| Isolate 1 | | |
| S. aureus | Methicillin-resistant strain of the USA300 | Strong |
| Clinical | clonal group; blood isolate | |
| Isolate 2 | | |
| S. aureus | Methicillin-resistant strain of the USA700 | Weak |
| Clinical | clonal group; cultured from deep would | |
| Isolate 3 | | |
| S. aureus | Methicillin-resistant strain of the USA200 | Strong |
| Clinical | clonal group; cultured from deep wound | |
| Isolate 4 | | |

¹Classification based on comparison of biofilm forming capacity and a biofilm positive control, S. epidermidis ATCC 12228; strong indicates biofilm ≧ than S. epidermidis and weak ≦ than the control as determined by microtiter plate assay.

Like in Example 1, biofilm formation was assessed under static conditions using polystyrene 96-well plates (Corning, Inc.). Overnight bacterial cultures were diluted to an OD₆₀₀ of 0.1 in TSB (˜10⁷ CFU/mL), and 20 μL were added to individual wells filled with 180 μL of media and incubated at 37° C. for 48 h. To assess the biofilm dispersal activity of D-AAs, the culture medium from biofilms was removed after 48 h and 200 μL fresh medium containing either an individual D-AA or an equimolar mixture of D-AAs (1:1:1: D-Met:D-Pro:D-Trp) were added at the indicated concentrations. After treatment with D-AA(s) for 24 h, plates were gently washed with 1× phosphate buffered saline (PBS) to remove unattached cells, stained with 0.1% (w/v) crystal violet (Sigma Aldrich) for 10 min, rinsed with PBS, and then solubilized with 80% (v/v) ethanol. Biofilm biomass was determined by measuring the absorbance of solubilized stain at 570 nm using a microtiter plate reader. For assays measuring the ability of D-AA to block biofilm formation, cells were grown under biofilm conditions as above in the presence of media containing D-AAs. Representative images of the plates of CV-stained biofilms following treatment with D-AA prior to solubilization were taken using a digital camera. All assays were repeated in triplicate with a minimum of four technical replicates.

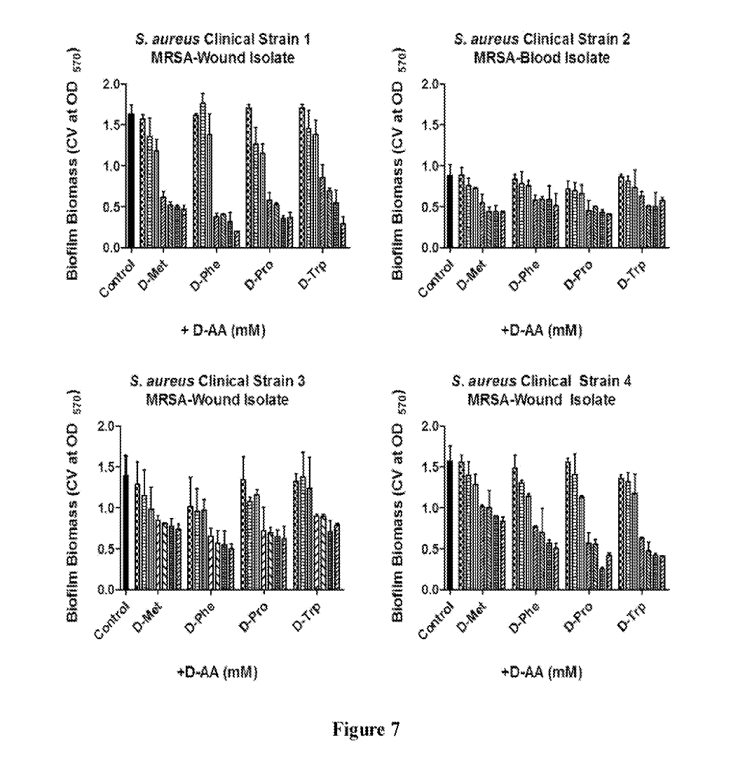

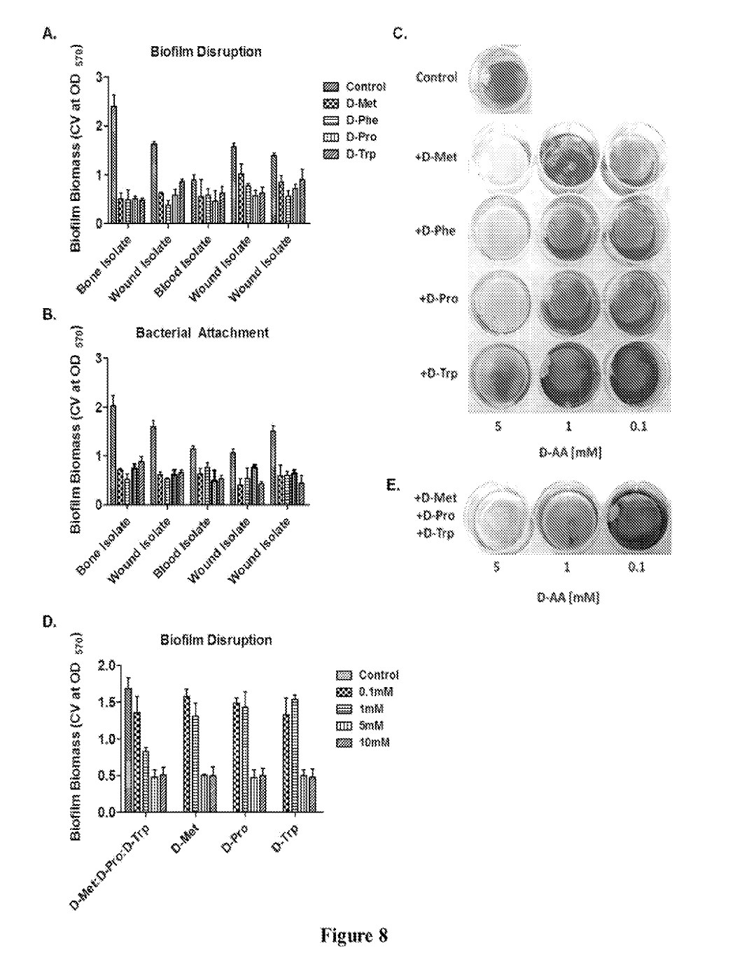

Furthermore, pre-screening of eight individual D-AAs identified four amino acids, including D-Met, D-Phe, D-Pro, and D-Trp, as effective at dispersing biofilms formed by the four clinical isolates (FIG. 7), whereas the other four D-AAs had minimal effects. D-AAs dispersed biofilms in a dose-responsive manner and were most effective at concentrations ≧5 mM. Thus, 5 mM was chosen as the concentration for future studies. The efficacy of D-AAs varied between different bacterial strains, although for each strain tested more than one of the four D-AAs was effective at dispersing biofilms. The anti-biofilm effect was isomer-specific, as no dispersal activity was observed with L-isomers of D-AAs. When tested against the panel of clinical isolates of methicillin-resistant S. aureus (n=5), D-Phe, D-Met, D-Trp, and D-Pro were effective at dispersing established biofilms in vitro as determined by the measurement of the biofilm biomass (FIGS. 8A and 8C). In addition to dispersing established biofilms, the four identified D-AAs also significantly blocked formation of biofilms by the clinical strains when bacteria were cultured in the presence of D-AAs (FIG. 8B). When combined as an equimolar mixture of D-Met, D-Pro, and D-Trp, biofilm-dispersive activity was enhanced (FIGS. 8D-8E), as suggested by the decrease in biofilm biomass observed at D-AA concentrations lower than 5 mM. Without being bound by theory or mechanism, it appeared that D-AAs primarily possessed a biofilm dispersal property and had little to no effect on the growth of the bacteria.

### Example 4

This Example describes procedures that characterize the cytotoxicity of the D-amino acids discussed in Example 3.

Human dermal fibroblasts and osteoblasts (PromoCell, Heidelberg, Germany) were maintained in Dulbecco's Modified Eagle Medium (DMEM; Invitrogen, Grand Island, N.Y.) supplemented with 10% Fetal Bovine Serum (FBS) and 1× penicillin/streptomycin at 37° C. in 5% CO₂. Prior to each assay, cells were seeded at 100% confluence in black-clear bottom 96-well plates. After 24 h cells were exposed to media containing D-AA (1 nM-50 mM) and incubated for 24 h. Following treatment, cells were washed, re-suspended in 100 μL of sterile saline, and assessed for viability using the CellTiter-Fluor Cell Viability Assay (Promega, Madison, Wis.) following the manufacturer's instructions. Viability assays were performed in triplicate with a minimum of four replicates. Viability was reported as the percentage of viable cells relative to untreated controls.

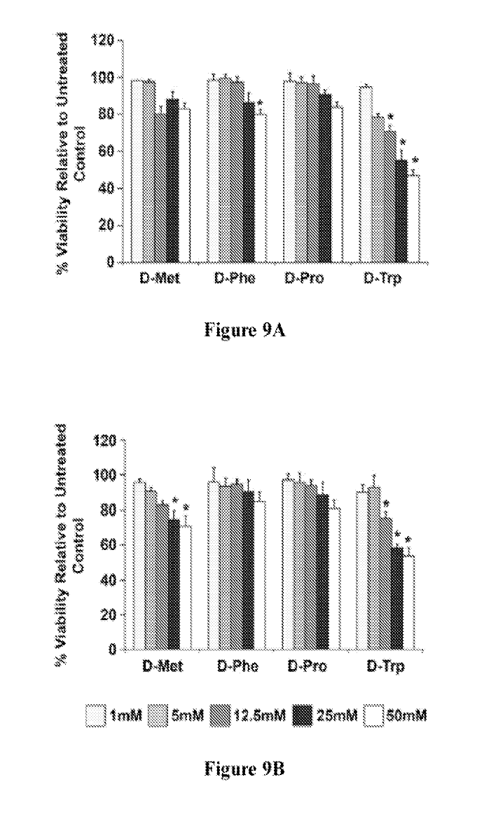

The human osteoblasts and dermal fibroblasts exposed to up to 50 mM of D-Met, D-Phe, D-Pro showed >70% viability after 24 h. Cytotoxicity was observed in mammalian cells exposed to D-Trp at concentrations exceeding 12.5 mM (˜60% viability) (FIGS. 9A-B). Thus, the D-AAs had little to no cytotoxic effects on mammalian cells at or above concentrations observed to be effective for preventing and disrupting biofilms in vitro.

### Example 5

This Example describes synthesizing composites that comprise polyurethane scaffolds that include the D-amino acids discussed in Example 3. This Example also characterizes the mechanical, release, and other properties of the composites.

For polyurethane scaffold synthesis, ε-caprolactone and stannous octoate (Sigma-Aldrich), glycolide and D,L-lactide (Polysciences), an isocyanate-terminated prepolymer (22.7% NCO) comprising polyethylene glycol (PEG) end-capped with lysine triisocyanate (LTI) at a 2:1 molar ratio of LTI:PEG (Medtronic), and triethylene diamine (TEGOAMIN 33, Evonik, Hopewell, Va.) were utilized. Polyester triols with a molecular weight of 900 g mol⁻¹ and a backbone comprising 60 wt % ε-caprolactone, 30% glycolide, and 10% lactide (T6C3G1L900) were synthesized as previously described. Appropriate amounts of dried glycerol and ε-caprolactone, glycolide, DL-lactide, and stannous octoate (0.1 wt-%) were mixed in a 100-ml flask and heated under an argon atmosphere with mechanical stirring to 140° C. for 24 h. The polyester triol was subsequently washed with hexane and dried. The appropriate amounts of each D-AA were pre-mixed. Next, the polyester triol, LTI-PEG prepolymer (excess isocyanate 15%), 2.0 parts per hundred parts polyol (pphp) tertiary catalyst, 3.0 pphp water, 4.0 pphp calcium stearate pore opener, and the equimolar mixture of D-AAs (0-10 wt % total D-AA, 1:1:1 mixture of D-Met:D-Pro:D-Trp; labile powder) were loaded into a 20 ml cup and mixed for 1 min using a Hauschild SpeedMixer DAC 150 FVZ-K vortex mixer (FlackTek, Landrum, S.C.). The reactive mixture was allowed to cure and foam at room temperature for 24 h. Cylindrical samples for in vivo testing (3 mm diameter×6.5 mm height) were cut using a coring tool and then sterilized by treating with ethylene oxide (EO).

Scaffold density was determined from mass and volume measurements of cured samples, from which the gravimetric porosity was calculated as the volume fraction of pores. After curing, PUR sections were sputter-coated with gold and imaged using a Hitachi 4200 SEM. Pore size was determined from the SEM images using MetaMorph 7.1 Image Analysis software (MDS Analytical Technologies, Sunnyvale, Calif.). Compressive mechanical properties of the scaffolds were measured using a TA Instruments Q800 Dynamic Mechanical Analyzer (DMA, New Castle, Del.). Samples were tested after 24 h or 7 days of incubation in PBS. Stress-strain curves were generated by compressing wet cylindrical 6 mm×6 mm samples at 37° C. at a rate of 0.1 N/min until they reached 60% strain. The compressive modulus was determined from the slope of the initial linear region of each stress-strain curve. Since the scaffolds could not be compressed to failure due to their elasticity, the compressive stress was reported at 50% strain.

SEM analysis was performed to examine the effect of augmentation with D-AAs on bacterial attachment and biofilm formation on the scaffold in vitro and in vivo. PUR scaffolds were fixed with 2% (w/v) glutaraldehyde, 2% (w/v) paraformaldehyde (PFA), 0.15M sodium cacodylate, 0.15% (w/v) alcian blue for 3 h, rinsed 3× with 0.15M sodium cacodylate buffer, and incubated in 1% (v/v) osmium tetroxide in sodium cacodylate for 1 h. Samples were dehydrated with a stepwise gradient of ethanol and then treated with hexamethyldisilizane prior to drying in a desiccator overnight. Samples were sputter-coated with gold palladium and viewed with a Hitachi 4200 or JEOL-6610 scanning electron microscope.

PUR scaffolds incorporating 10 wt % of a 1:1:1 mixture of D-Met:D-Pro:D-Trp were incubated in PBS at 37° C. under static conditions for up to 8 weeks. This mixture was evaluated in vitro, in vivo and in the release kinetics tests to account for potential interactions between D-AAs during release. The medium was sampled on days 1, 2, 4, 7, 10, 14, 21, and 28 and analyzed for D-AAs by HPLC using a system equipped with a Waters 1525 binary pump and a 2487 Dual-Absorbance Detector at 200 nm. Samples of released D-AAs were eluted through an Atlantis HILIC Silica column (5 μm particle size, 4.6 mm diameter×250 mm length) using an isocratic mobile phase flowing at 1 mL/min. The mobile phase contained 2.5 mM potassium dihydrogen phosphate with pH=2.85 (A) and Acetonitrile (B) at a ratio of A25:B75. The column oven temperature was maintained at 30° C. Sample concentration was determined in reference to an external standard curve using the Waters Breeze system. Standard curves were prepared in the following concentration ranges: (1) 7.8 μg/mL to 1 mg/mL for D-Met and D-Pro and (2) 0.78 μL/mL to 100 μg/mL for D-Trp.

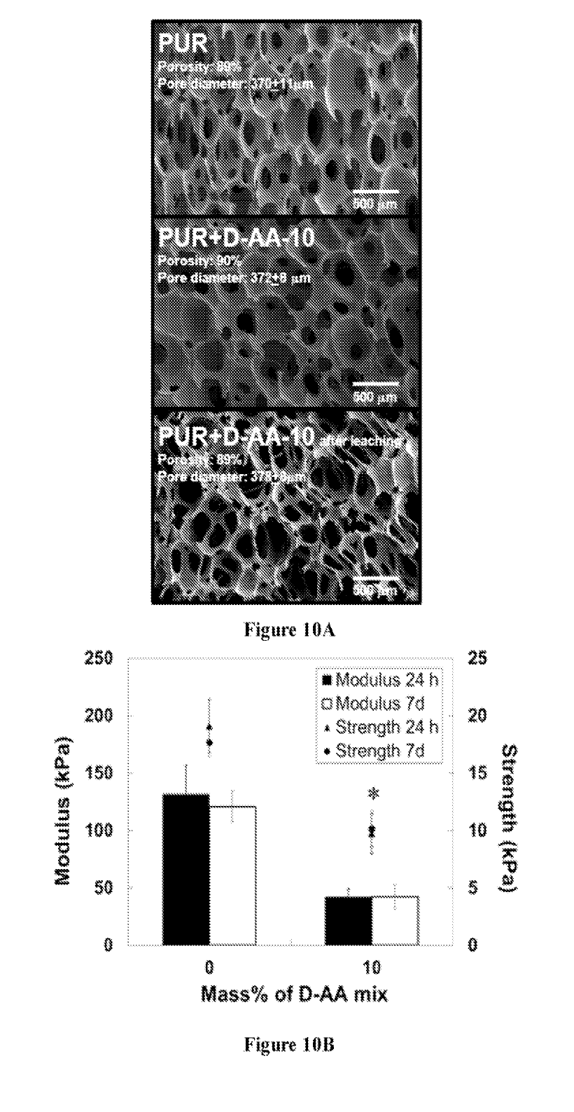

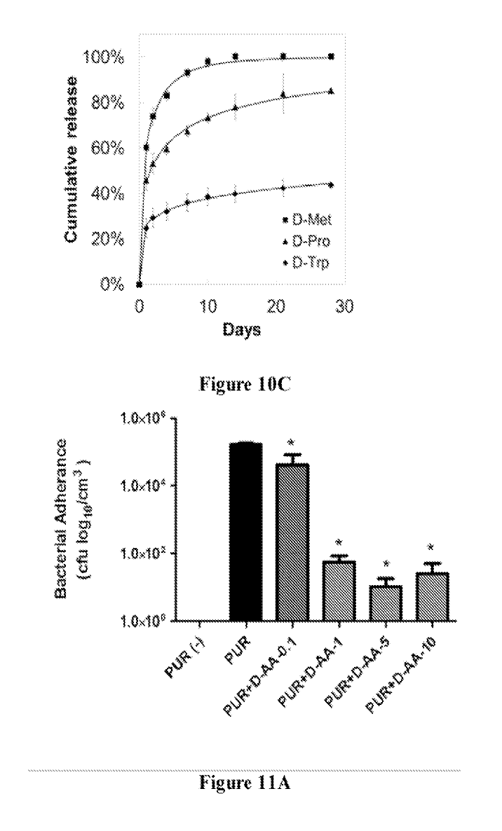

Scaffolds containing 0 (PUR) or 10 wt % D-AA mixture (PUR+D-AA-10) had similar values of density, porosity, and pore size before and after leaching overnight in PBS. Representative SEM images of the PUR and PUR+D-AA-10 scaffolds show inter-connected pores and a mean pore diameter ranging from 370 to 378 μm (FIG. 10A). While the addition of 10% D-AA mix to the PUR scaffolds did not have a large effect on the pore size or the porosity, the wet mechanical properties were reduced compared to the empty scaffold (FIG. 10B). There were no notable differences in the properties of the scaffolds incubated in PBS for 24 hours or 7 days. Thus, while the modulus decreased significantly even after 24 h, at which time only a fraction of the D-AAs had released, it experienced little to no decrease for up to 7 days of incubation. Without being bound by theory or mechanism, these observations suggest that the decrease in modulus with addition of D-AAs results from defects in the pore walls of the scaffold caused by the presence of the particles rather than from the formation of new pores due to leaching of the D-AAs.

The release kinetics of D-Pro, D-Met, and D-Trp were characterized by an initial burst followed by a sustained release for up to 21 days (FIG. 10C). D-Met released the fastest, characterized by a 60% burst on day 1 and nearly 100% release by day 14. The release of D-Pro was somewhat slower (45% burst and 85% release by day 28), while D-Trp released even more slowly (25% burst and 44% release by day 28). The Weibull equation has been used to identify the mechanism controlling drug release from polymeric materials:

$$\frac{{M}_{t}}{M} = 1 - exp ( - {at}^{b} )$$

where Mₜ corresponds to the mass of drug released in time t, M_{∞} is the mass of drug released at infinite time (i.e., initial loading of drug), and a and b are constants. When b<0.75, Fickian diffusion controls drug release, while a mechanism involving both diffusion and swelling controls release when b>0.75. The D-AA release data were fit to the Weibull model and the values of the b parameter for D-Met, D-Pro, and D-Trp were calculated as 0.56, 0.35, and 0.21 respectively, suggesting that the release of each D-AA from the scaffolds was diffusion-controlled. Specifically, at 4 weeks, more than 85% of D-Pro and D-Met had been released and less than 10% of the scaffold had degraded, which is consistent with the notion that D-AA release was diffusion-controlled at early time points. However, less than 40% of the D-Trp had been released by 4 weeks, suggesting that degradation of the scaffold may control D-Trp release kinetics at later (e.g., after 4 weeks) time points.

### Example 6

This Example describes procedures conducted to characterize the effects on biofilm-dispersion caused by the composites of Example 5.

Bacterial adherence and biofilm formation on scaffolds with or without D-AAs was evaluated as described previously (Table 6). Sterile blank PUR scaffolds with no D-AAs were utilized as a negative control (PUR(−)). Blank scaffolds (denoted as PUR) or scaffolds augmented with an equimolar mixture of D-AAs (denoted as PUR+D-AA-x, where x=0.1, 1.0, 5.0, or 10 wt % 1:1:1 mixture of D-Met:D-Pro:D-Trp) were placed into 24-well polystyrene plates containing sterile PBS for 2 h at room temperature. Samples were then transferred into a bacterial suspension of UAMS-1 (10⁷ CFU/mL) in PBS and exposed for an additional 2 h at 37° C. with agitation in 24-well plates. Following exposure, scaffolds were rinsed with PBS to remove non-attached bacteria and incubated overnight in PBS at 37° C. to allow adequate time for attached bacteria to develop biofilms. Following incubation, scaffolds were then placed in 1 mL PBS and sonicated for 10 min using a low-power bath sonicator. Bacterial CFUs per gram of scaffold were determined by plating serial dilutions on blood agar plates. Bacterial attachment and biofilm formation on scaffolds following incubation was also evaluated by SEM analysis.

TABLE 6
Investigated composites with and without D-amino acids.

| Group | Description | 24 h |
|---|---|---|
| PUR (−) | Sterile blank PUR scaffold w/o D-AAs (negative | 4 |
| | control) | |
| PUR | Contaminated blank PUR scaffold | 4 |
| PUR + | Contaminated blank PUR scaffold with 0.1% D-AAs | 4 |
| D-AA-0.1 | | |
| PUR + | Contaminated blank PUR scaffold with 1.0% D-AAs | 4 |
| D-AA-1 | | |
| PUR + | Contaminated blank PUR scaffold with 5.0% D-AAs | 4 |
| D-AA-5 | | |
| PUR + | Contaminated blank PUR scaffold with 10% D-AAs | 4 |
| D-AA-10 | | |

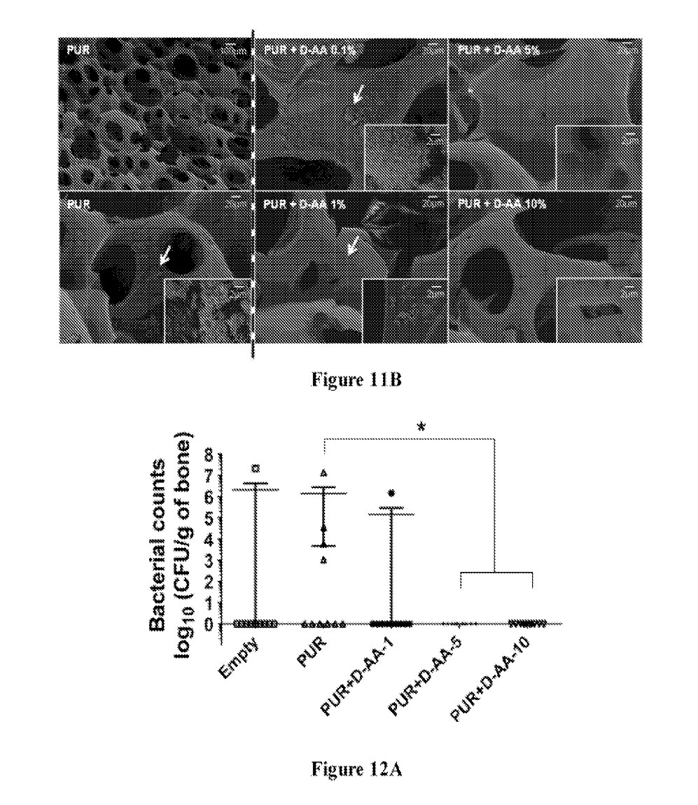

Incorporation of D-AA into PUR scaffolds at concentrations of at least 1 wt % D-AA reduced the amount of attached bacteria and biofilm formation on the surface compared to the scaffolds without D-AA. PUR scaffolds with 1, 5, and 10 wt % D-AA had a at least 4-log reduction in the number of bacteria (FIG. 11A), while PUR scaffolds with 0.1% showed a more moderate (˜1-log reduction) but a reduction in bacteria attached to the scaffold surface. Consistent with the bacterial counts, SEM images of PUR scaffolds augmented with D-AA also demonstrated the reduction in surface-attached bacteria within biofilms on scaffolds augmented with the D-AA mixture (FIG. 11B). As indicated by the bacterial counts, PUR scaffolds without D-AA or with 0.1% D-AA had extensive bacterial colonization and the presence of biofilms on the surface.

### Example 7

This Example describes the administration of the composites of Example 5 in a rat model, and also discusses the ability of the scaffolds to disperse biofilms and treat wounds in the rat models.

A contaminated critical size defect in rat (Sprague-Dawley; 373±4.15 g) femurs was again utilized as the in vivo model of infection (Table 7). Briefly, a 6-mm segmental defect was created using a small reciprocating saw blade (MicroAire 1025, MicroAire, Charlottesville, Va.), stabilized with a polyacetyl plate (length 25 mm, width 4 mm and height 4 mm) and fixed to the surface of the femur using threaded K-wires. Blank PUR scaffolds implanted in a sterile defect were utilized as a negative control (PUR(−)) and for SEM analysis to distinguish between host cellular and bacterial infiltration of the scaffolds. The defects in all other animals were then implanted with 30 mg of type I bovine collagen (Stryker Biotech, Hopkinton, Mass.) wetted with 10² CFU of S. aureus strain Xenogen-36 (Caliper Life Science) or S. aureus strain UAMS-1. Six hours after contamination, the wounds were opened, debrided, and irrigated with saline. PUR or PUR+D-AA-x (1.0, 5.0, or 10 wt % 1:1:1 mixture of D-Met:D-Pro:D-Trp) scaffolds were then implanted into the wounds. Since cefazolin is desirable for certain primary prevention of infections associated with open fractures, rats received systemic antimicrobial treatment with cefazolin (5 mg/kg) administered subcutaneously for 3-days post-surgery. Two weeks following surgery, the femurs were weighed, snap-frozen in liquid nitrogen, ground to a fine powder, and re-suspended in saline. CFUs (expressed as log₁₀ CFU/g tissue) were determined by plating serial dilutions onto blood agar plates and incubated at 37° C. for 24 h. PUR scaffolds from sterile defects (PUR(−), negative control) as well as blank PUR and PUR+D-AA-10% scaffolds from contaminated defects were evaluated by SEM.

TABLE 7
Investigated composites in 6-mm segmental defect in rat
femora. Outcomes were assessed at 2 weeks (n = 10).

| | | No | | |
|---|---|---|---|---|
| | | Infec- | UAMS- | |
| Group | Description | tion | 1 | XEN36 |
| PUR (−) | Blank PUR scaffold in sterile | 10 | 0 | 0 |
| | defect (negative control) | | | |
| Empty | Contaminated defect not grafted | 0 | 10 | 10 |
| | with scaffold (positive control) | | | |
| PUR | Blank PUR scaffold in | 0 | 10 | 10 |
| | contaminated defect | | | |
| PUR + | PUR scaffold with 1.0% D-AAs in | 0 | 10 | 0 |
| D-AA-1 | contaminated defect | | | |
| PUR + | PUR scaffold with 5.0% D-AAs in | 0 | 10 | 0 |
| D-AA-5 | contaminated defect | | | |
| PUR + | PUR scaffold with 10% D-AAs in | 0 | 10 | 10 |
| D-AA-10 | contaminated defect | | | |

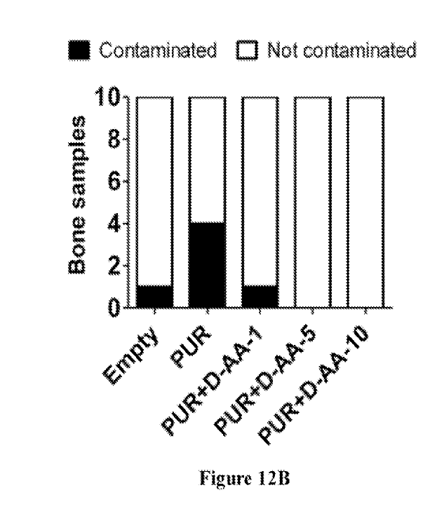

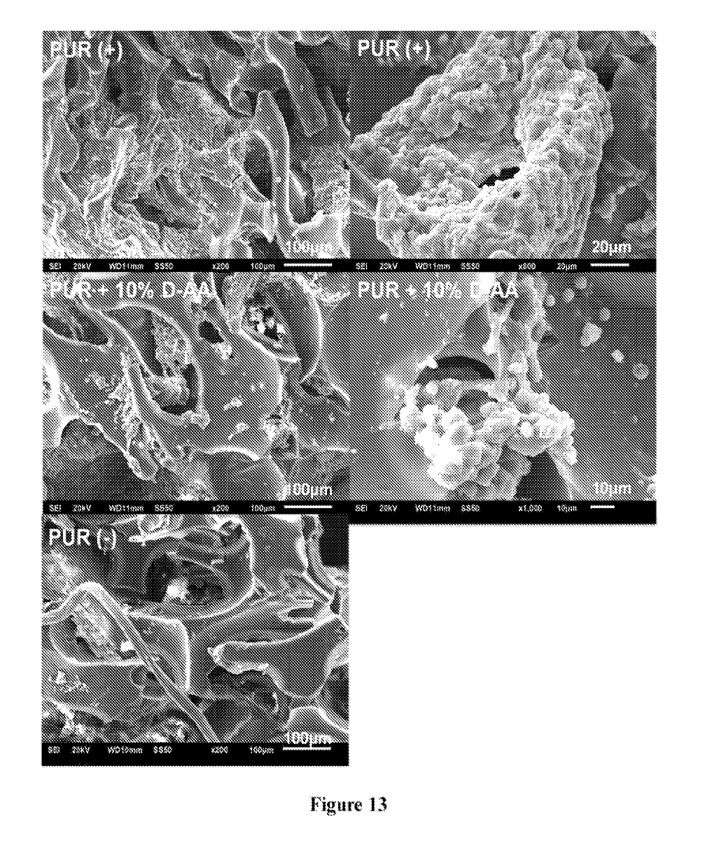

Treatment of femoral UAMS-1-contaminated defects with PUR+D-AA-5 or PUR+D-AA-10 reduced bacterial contamination within the homogenized bone (p<0.05) (FIG. 12A), while lower doses did not reduce contamination compared to the empty (untreated) defect control. Similarly, PUR+D-AA-5 and PUR+D-AA-10 reduced the number of contaminated samples compared to the PUR scaffold (FIG. 12B) (p=0.087). Consistent with these observations, SEM analysis of scaffolds removed from rats following infection showed a reduction of bacteria attached to the surface of the scaffolds (FIG. 13). Blank PUR scaffolds implanted in contaminated defects exhibited bacterial adhesion and biofilm formation on the majority of the surface, whereas PUR+D-AA-10 showed a reduction in the amount of attached bacteria. In contrast, PUR+D-AA scaffolds implanted in defects contaminated with 10² CFUs Xen-36 strain, a weak biofilm producer, did not significantly reduce bacterial contamination or the number of contaminated samples compared to the empty defect.

### Example 8

This Example describes procedures conducted to synthesize and characterize composites comprising polyurethane grafts, D-amino acids, and synthetic tissue substitutes. To avoid undue repetition, the materials, methods, or the like that are repeated from prior Examples have been omitted.

PUR grafts (LV) comprising a 450 g mol⁻¹ polyester triol (70 wt %-caprolactone, 20% glycolide, and 10% lactide (T7C2G1L450), an LTI-PEG prepolymer (29.0% NCO), triethylene diamine, and 45 wt % MasterGraft (MG, 85% β-tricalcium phosphate/15% hydroxyapatite) were injected into sterile bilateral 11×18 mm plug defects in the femoral condyles of sheep. The composite expanded and set in 10 minutes to form a composite with 45% porosity and 18 vol % MG.

Two treatment groups were investigated: PUR/MG and PUR/MG further comprising D-AA (10 wt % 1:1:1 D-Pro:D-Met:D-Phe) (LV/MG+DAA). Femoral condyles were harvested at 16 weeks and evaluated for new bone formation by μCT (FIGS. 14A and 14B). A cross-sectional photograph of the LV/MG graft (FIG. 14C) shows new bone (NB) forming at the interface between the host bone (HB) and the acellular graft (LV) at the inner core. BV/TV was measured in 4 annular regions extending the along the length of the implant (FIG. 14D, inset). BV/TV increased from values comparable to that of LV/MG alone (18-25 vol %) in the inner core to 42-52 vol % near the host bone interface (outer core). There were no significant differences between groups. Thus, the D-AAs appeared to exhibit low cytotoxicity toward host cells in the bone microenvironment, and local delivery of D-AAs from the injectable bone graft did not hinder new bone formation.

### Example 9

This Example describes procedures conducted to synthesize and characterize composites comprising D-amino acids that can be used to treat cutaneous wounds. The composites of this Example comprised a collagen sponge tissue graft. To avoid undue repetition, the materials, methods, or the like that are repeated from prior Examples have been omitted.

6-mm excisional wounds in C57BL/6 mice (6-8 weeks), which had previously been shaved and sterilized, were made. The mice were contaminated with 2×10⁶ or 2×10⁷ CFU S. aureus Xen 36A (Perkin Elmer, Waltham, Mass.). Approximately 2-3 minutes following exposure to bacteria, collagen gels (hydrogel, HG) with and without 5% D-Trp were applied to the wounds for about 5 minutes before applying a Tegaderm bandage (3M, St. Paul, Minn.). Wounds were monitored daily for up to 3 days. At 1 and 3 days post-infection mice were evaluated using the IVIS (to visualized bioluminescent bacteria within wounds) and the wound tissues were harvested, homogenized, and plated on blood agar to enumerate bacteria within wound (CFU/mL/g tissue).

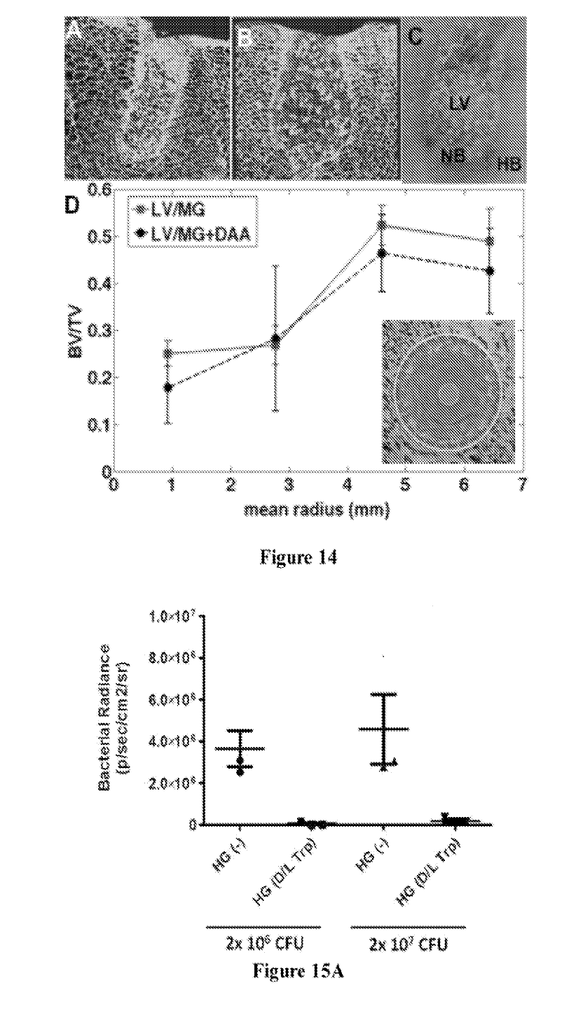

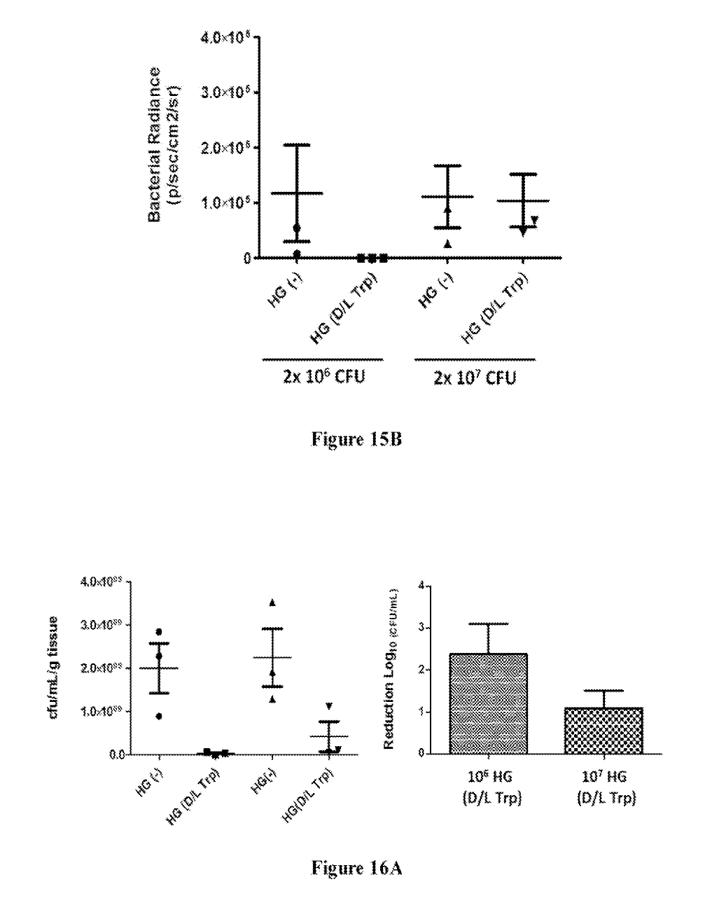

The gel supported a bolus release of D-Trp (about 80% release in 1 day), which was completely released by 3 days. At 2×10⁶ CFU, D-Trp reduced contamination (assessed by bioluminescence imaging and bacterial counts) at both 1 and 3 days (not shown). Furthermore, FIGS. 15 and 16 show that the collagen sponges comprising the D-AA resulted in lower amounts of bacteria in the wounds following treatment when compared to blank collagen sponges.

### Example 10

This Example describes procedures conducted to synthesize and characterize composites comprising demineralized bone matrix (DBM) and D-amino acids. To avoid undue repetition, the materials, methods, or the like that are repeated from prior Examples have been omitted.

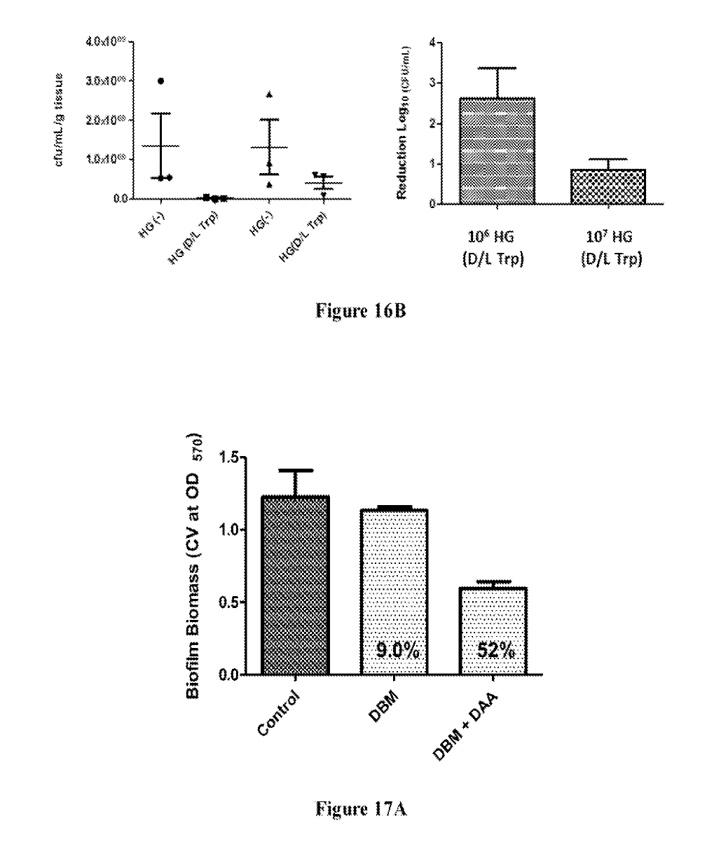

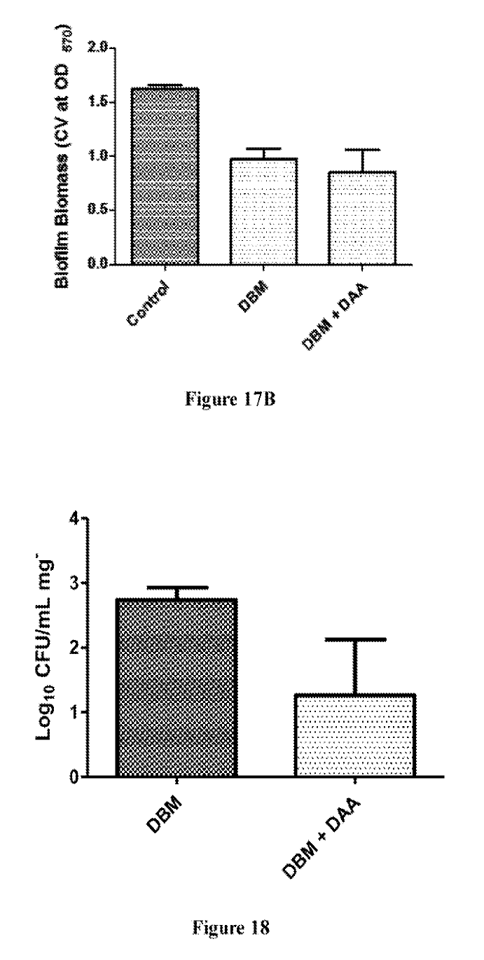

Briefly, DBM was washed with a 1:1:1 mixture of D-Phe, D-Met, and D-Pro so that the resulting DBM comprised 10% w/w of the D-amino acids. Equally cut sections of either empty DBM (DBM) or DBM incorporating the D-AA mixture (DBM+DAA) were then placed into a 12-well plate containing 1 mL of 10⁵ bacteria, specifically either MSSA or MRSA, diluted in Tryptic Soy Broth (TSB). Plates were incubated under static conditions at 37° C. for 24 h. Following incubation, plates were washed with PBS, stained with 0.1% crystal violet, and biofilm biomass was determined by measuring the optical density (OD_{570 nm}) following solubilization of CV in 80% EtOH. Control is a biofilm established in the 12 well plates in the absence of DBM. As shown in FIG. 17, the DBM comprising D-AA was capable of decreasing the biofilm biomass present relative to DBM that did not comprise any additional components.

The DBM with and without the D-AA mixture of D-Phe, D-Met, and D-Pro (10% w/w) then placed into a 12-well plate containing 1 mL of 10⁴ bacteria diluted in Tryptic Soy Broth (TSB). Plates were incubated with moderate agitation at 37° C. for 2 h. Following incubation, supernatants containing DBM were aspirated and passed through a nylon cell strainer (40 μM) and the bone matrix was gently washed with PBS. Remaining DBM was then resuspended in about 1 mL of PBS and serial dilutions were performed to enumerate attached bacterium. Values reported as Log₁₀ CFU/mL per mg of dry DBM are shown in FIG. 18. The results indicate that the incorporation of D-AA into tissue grafts such as DBM can reduce bacterial attachment.

The invention thus being described, it will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the Specification, including the Examples, be considered as exemplary only, and not intended to limit the scope and spirit of the invention.

While the terms used herein are believed to be well understood by one of ordinary skill in the art, the definitions set forth herein are provided to facilitate explanation of the presently-disclosed subject matter. Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are described above.

Following long-standing patent law convention, the terms “a”, “an”, and “the” refer to “one or more” when used in this application, including the claims. Thus, for example, reference to “an amino acid” includes a plurality of such amino acids, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±50%, in some embodiments ±40%, in some embodiments ±30%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Throughout this application, various publications are referenced. All such references, specifically including the four lists below, are incorporated herein in their entirety by this reference.

### REFERENCES

1. R. B. Gustilo and J. T. Anderson, Journal of Bone & Joint Surgery, American volume, 84, 682 (2002).
2. R. B. Gustilo, R. L. Merkow and D. Templeman, Journal of Bone & Joint Surgery, 72, 299-304 (1990).
3. C. M. Kelly, R. M. Wilkins, S. Gitelis, C. Hartjen, J. T. Watson and P. T. Kim, Clinical Orthopaedics & Related Research, 382, 42-50 (2001).
4. W. W. Rittmann and S. M. Perren, Cortical bone healing after internal fixation and infection, Springer, New York (1974).
5. P. Worlock, R. Slack, L. Harvey and R. Mawhinney, Injury, 25, 31-38 (1994).
6. A. A. Beardmore, D. E. Brooks, J. C. Wenke and D. B. Thomas, Journal of Bone & Joint Surgery, 87A, 107-112 (2005).
7. J. Calhoun and J. Mader, Clinical Orthopaedics & Related Research, 341, 206-214 (1997).
8. L. Dahners and C. Funderburk, Clinical Orthopaedics & Related Research, 219, 278-282 (1987).
9. J. Humphrey, S. Mehta, A. Seaber and T. Vail, Clinical Orthopaedics & Related Research, 349, 218-224 (1998).
10. K. Kanellakopoulou and E. J. Giamarellos-Bourboulis, Drugs, 59, 1223-1232 (2000).
11. J. T. Mader, J. Calhoun and J. Cobos, Antimicrobial Agents and Chemotherapy, 41, 415-418 (1997).
12. J. Rogers-Foy, D. Powers, D. Brosnan, S. Barefoot, R. Friedman and M. LaBerge, Journal of Investigative Surgery, 12, 263-275 (1999).
13. H. Buchholz, R. Elson and K. Heinert, Clinical Orthopaedics & Related Research, 190, 96-108 (1984).
14. P. Osterman, D. Seligson and S. Henry, Journal of Bone & Joint Surgery, British volume, 77, 93-97 (1995).
15. S. K. Seeley, J. V. Seeley, P. Telehowski, S. Martin, M. Tavakoli, S. L. Colton, B. Larson, P. Forrester and P. J. Atkinson, Clinical Orthopaedics & Related Research, 420, 298-303 (2004).
16. C. M. Stevens, K. D. Tetsworth, J. H. Calhoun and J. T. Mader, Journal of Orthopaedic Research, 23, 27-33 (2005).
17. S. Gitelis and G. Brebach, Journal of Orthopaedic Surgery (Hong Kong), 10, 53-60 (2002).
19. A. C. McLaren, Clinical Orthopaedics & Related Research, 427, 101-106 (2004).
20. C. G. Ambrose, G. R. Gogola, T. A. Clyburn, K. A. Raymond, A. S. Peng and A. G. Mikos, Clinical Orthopaedics & Related Research, 415, 279-285 (2003).
21. S. Gogolewski and K. Goma, Journal of Biomedical Materials Research Part A, 80A, 94-101 (2007).
22. J. Guan, K. L. Fujimoto, M. S. Sacks and W. R. Wagner, Biomaterials, 26, 3961-3971 (2005).
26. J. Guan, J. J. Stankus and W. R. Wagner, Journal of Controlled Release, 120, 70-78 (2007).
27. R. Adhikari and P. A. Gunatillake, Biodegradable polyurethane/urea compositions, US Patent No. 2005/0238,683 (2004).
28. A. S. Sawhney and J. A. Hubbell, Journal of Biomedical Materials Research, 24, 1397-1411 (1990).
29. ASTM-International, D3574-05. Standard test methods for flexible cellular materials—slab, bonded, and molded urethane foams, pp 360-368 (2007).
30. G. Oertel, Polyurethane Handbook, Hanser Gardner Publications, Berlin (1994).
31. M. C. Caturla, E. Cusido and D. Westerlund, Journal of Chromatography A, 593, 69-72 (1992).
32. ASTM-International, D695-02a. Standard Test Method for Compressive Properties of Rigid Plastics (2007).
33. J. E. Mark, E. Erman and F. R. Eirich, Eds. Science and Technology of Rubber, Academic Press, Inc., San Diego, Calif. (1994).
34. C. G. Ambrose, T. A. Clyburn, K. Louden, J. Joseph, J. Wright, P. Gulati, G. R. Gogola and A. G. Mikos, Clinical Orthopaedics & Related Research, 421, 293-299 (2004).
35. M. Hombreiro-Pérez, J. Siepmann, C. Zinutti, A. Lamprecht, N. Ubrich, M. Hoffman, R. Bodmeier and P. Maincent, Journal of Controlled Release, 88, 413-428 (2003).
36. J.-Y. Zhang, B. A. Doll, E. J. Beckman and J. O. Hollinger, Tissue Engineering, 9, 1143-1157 (2003).
37. Chen X, et al. J Orthop Res. 2005; 23(4):816-23.
38. Gogolewski S, Gorna K, Turner A S. J Biomed Mater Res A. 2006; 77A(4):802-10.
39. Ambrose C G, et al. Clin Orthop Relat Res. 2003; 415:279-85.
40. Lew D P, Waldvogel F A. Osteomyelitis. Lancet 2004; 364:369-79.
41. Conterno L O, da Silva Filho C R. Antibiotics for treating chronic osteomyelitis in adults. Cochrane Database Syst Rev 2009:CD004439.
42. Huh J, Stinner D J, Burns T C, Hsu J R. Infectious complications and soft tissue injury contribute to late amputation after severe lower extremity trauma. J Trauma 2011; 71:S47-51.
43. Costerton J W. Biofilm theory can guide the treatment of device-related orthopaedic infections. Clin Orthop Relat Res 2005:7-11.
44. Costerton J W, Stewart P S, Greenberg E P. Bacterial biofilms: a common cause of persistent infections. Science 1999; 284:1318-22.
45. Brady R A, Leid J G, Calhoun J H, Costerton J W, Shirtliff M E. Osteomyelitis and the role of biofilms in chronic infection. FEMS Immunol Med Microbiol 2008; 52:13-22.
46. Marrie T J, Costerton J W. Mode of growth of bacterial pathogens in chronic polymicrobial human osteomyelitis. J Clin Microbiol 1985; 22:924-33.
47. Gristina A G, Oga M, Webb L X, Hobgood C D. Adherent bacterial colonization in the pathogenesis of osteomyelitis. Science 1985; 228:990-3.
48. Costerton J W. Introduction to biofilm. International journal of antimicrobial agents 1999; 11:217-21; discussion 37-9.
49. del Pozo J L, Patel R. The challenge of treating biofilm-associated bacterial infections. Clin Pharm. Therap 2007; 82:204-9.
50. Hall-Stoodley L, Costerton J W, Stoodley P. Bacterial biofilms: from the natural environment to infectious diseases. Nat Rev Microbiol 2004; 2:95-108.
51. Esteban J, Molina-Manso D, Spiliopoulou I, Cordero-Ampuero J, Fernandez-Roblas R, Foka A, Gomez-Barrena E. Biofilm development by clinical isolates of Staphylococcus spp. from retrieved orthopedic prostheses. Acta Orthop 2010; 81:674-9.
52. O'Neill E, Pozzi C, Houston P, Smyth D, Humphreys H, Robinson D A, O'Gara J P. Association between methicillin susceptibility and biofilm regulation in Staphylococcus aureus isolates from device-related infections. J Clin Microbiol 2007; 45:1379-88.
53. Sanchez C J, Jr., Mende K, Beckius M L, Akers K S, Romano D R, Wenke J C, Murray C K. Biofilm formation by clinical isolates and the implications in chronic infections. BMC Infect Dis 2013; 13:47.
54. Palmer M, Costerton W, Sewecke J, Altman D. Molecular techniques to detect biofilm bacteria in long bone nonunion: a case report. Clin Orthop Relat Res 2011; 469:3037-42.
55. McDougald D, Rice S A, Barraud N, Steinberg P D, Kjelleberg S. Should we stay or should we go: mechanisms and ecological consequences for biofilm dispersal. Nat Rev Microbiol 2012; 10:39-50.
56. Hall-Stoodley L, Stoodley P. Biofilm formation and dispersal and the transmission of human pathogens. Trends Microbiol 2005; 13:7-10.
57. Kaplan J B. Biofilm dispersal: mechanisms, clinical implications, and potential therapeutic uses. J Dent Res 2010; 89:205-18.
58. Lynch A S, Abbanat D. New antibiotic agents and approaches to treat biofilm-associated infections. Expert opinion on therapeutic patents 2010; 20:1373-87.
59. Folsom J P, Baker B, Stewart P S. In vitro efficacy of bismuth thiols against biofilms formed by bacteria isolated from human chronic wounds. J Appl Microbiol 2011; 111:989-96.
60. Kaplan J B, LoVetri K, Cardona S T, Madhyastha S, Sadovskaya I, Jabbouri S, Izano E A. Recombinant human DNase I decreases biofilm and increases antimicrobial susceptibility in staphylococci. J Antibiotics 2012; 65:73-7.
61. Dow J M, Crossman L, Findlay K, He Y Q, Feng J X, Tang J L. Biofilm dispersal in Xanthomonas campestris is controlled by cell-cell signaling and is required for full virulence to plants. Proc Natl Acad Sci USA 2003; 100:10995-1000.
62. Jennings J A, Courtney H S, Haggard W O. Cis-2-decenoic acid inhibits S. aureus growth and biofilm in vitro: a pilot study. Clin Orthop Relat Res 2012; 470:2663-70.
63. Simonetti O, Cirioni O, Ghiselli R, Goteri G, Scalise A, Orlando F, et al. RNAIII-inhibiting peptide enhances healing of wounds infected with methicillin-resistant Staphylococcus aureus. Antimicrob Agents Chemother 2008; 52:2205-11.
64. Brackman G, Cos P, Maes L, Nelis H J, Coenye T. Quorum sensing inhibitors increase the susceptibility of bacterial biofilms to antibiotics in vitro and in vivo. Antimicrob Agents Chemother 2011; 55:2655-61.
65. Kolodkin-Gal I, Romero D, Cao S, Clardy J, Kolter R, Losick R. D-amino acids trigger biofilm disassembly. Science 2010; 328:627-9.
66. Hochbaum A I, Kolodkin-Gal I, Foulston L, Kolter R, Aizenberg J, Losick R Inhibitory effects of D-amino acids on Staphylococcus aureus biofilm development. J Bacteriol 2011; 193:5616-22.
67. Ercal N, Luo X, Matthews R H, Armstrong D W. In vitro study of the metabolic effects of D-amino acids. Chirality 1996; 8:24-9.
68. Smeltzer M S, Thomas J R, Hickmon S G, Skinner R A, Nelson C L, Griffith D, et al. Characterization of a rabbit model of staphylococcal osteomyelitis. J Orthop Res 1997; 15:414-21.
69. Weiss E C, Zielinska A, Beenken K E, Spencer H J, Daily S J, Smeltzer M S. Impact of sarA on daptomycin susceptibility of Staphylococcus aureus biofilms in vivo. Antimicrob Agents Chemother 2009; 53:4096-102.
70. Cassat J E, Lee C Y, Smeltzer M S. Investigation of biofilm formation in clinical isolates of Staphylococcus aureus. Methods Mol Biol 2007; 391:127-44.
71. Christensen G D, Simpson W A, Younger J J, Baddour L M, Barrett F F, Melton D M, Beachey E H. Adherence of coagulase-negative staphylococci to plastic tissue culture plates: a quantitative model for the adherence of staphylococci to medical devices. J Clin Microbiol 1985; 22:996-1006.
72. Guelcher S A, Patel V, Gallagher K M, Connolly S, Didier J E, Doctor J S, Hollinger J O. Synthesis and in vitro biocompatibility of injectable polyurethane foam scaffolds. Tissue Eng 2006; 12:1247-59.
73. Sawhney A S, Hubbell J A. Rapidly degraded terpolymers of dl-lactide, glycolide, and epsilon-caprolactone with increased hydrophilicity by copolymerization with polyethers. J Biomed Mater Res 1990; 24:1397-411.
74. Guelcher S, Srinivasan A, Hafeman A, Gallagher K, Doctor J, Khetan S, et al. Synthesis, In vitro degradation, and mechanical properties of two-component poly(ester urethane)urea scaffolds: Effects of water and polyol composition. Tis Eng. 2007; 13:2321-33.
75. Hafeman A, Li B, Yoshii T, Zienkiewicz K, Davidson J, Guelcher S. Injectable biodegradable polyurethane scaffolds with release of platelet-derived growth factor for tissue repair and regeneration. Pharm Res 2008; 25:2387-99.
76. Bhandare P, Madhavan P, Rao B M, Someswar Rao N. Determination of amino acid without derivatization by using HPLC-HILIC column. J Chem Pharm Res 2010; 2:372-80.
77. Kinnari T J, Esteban J, Martin-de-Hijas N Z, Sanchez-Munoz O, Sanchez-Salcedo S, Colilla M, Vallet-Regi M, et al. Influence of surface porosity and pH on bacterial adherence to hydroxyapatite and biphasic calcium phosphate bioceramics. J Med Microbiol 2009; 58:132-7.
78. Li B, Brown K V, Wenke J C, Guelcher S A. Sustained release of vancomycin from polyurethane scaffolds inhibits infection of bone wounds in a rat femoral segmental defect model. J Control Release 2010; 145:221-30.
79. Guelcher S A, Brown K V, Li B, Guda T, Lee B H, Wenke J C. Dual-purpose bone grafts improve healing and reduce infection. J Ortho Trauma 2011; 25:477-82.
80. Penn-Barwell J G, Murray C K, Wenke J C. Early antibiotics and debridement independently reduce infection in an open fracture model. J Bone Joint Sur Br 2012; 94-B:1-6.
81. Hospenthal D R, Murray C K, Andersen R C, Bell R B, Calhoun J H, Cancio L C, et al. Guidelines for the prevention of infections associated with combat-related injuries: 2011 update: endorsed by the Infectious Diseases Society of America and the Surgical Infection Society. J Trauma 2011; 71:S210-34.
82. Papadopoulou V, Kosmidis K, Vlachou M, Macheras P. On the use of the Weibull function for the discernment of drug release mechanisms. Int J Pharm 2006; 309:44-50.
83. Li B, Yoshii T, Hafeman A E, Nyman J S, Wenke J C, Guelcher S A. The effects of rhBMP-2 released from biodegradable polyurethane/microsphere composite scaffolds on new bone formation in rat femora. Biomaterials 2009; 30:6768-79.
84. mc M, Wild L, Schemitsch E, Waddell J. The use of an antibioticimpregnated, osteoconductive, bioabsorbable bone substitute in the treatment of infected long bone defects: early results of a prospective trial. J Orthop Trauma 2002; 16:622-7.
85. Radin S, Ducheyne P, Kamplain T, Tan B H. Silica sol-gel for the controlled release of antibiotics. I. Synthesis, characterization, and in vitro release. J Biomed Mater Res 2001; 57:313-20.
86. Noel S P, Courtney H S, Bumgardner J D, Haggard W O. Chitosan sponges to locally deliver amikacin and vancomycin: a pilot in vitro evaluation. Clin Orthop Relat Res 2010; 468:2074-80.
87. Feng K, Sun H, Bradley M A, Dupler E J, Giannobile W V, Ma P X. Novel antibacterial nanofibrous PLLA scaffolds. J Cont Rel. 2010; 146:363-9.
88. Virto M R, Elorza B, Torrado S, Elorza Mde L, Frutos G. Improvement of gentamicin poly(D,L-lactic-co-glycolic acid) microspheres for treatment of osteomyelitis induced by orthopedic procedures. Biomaterials 2007; 28:877-85.
89. Radin S, Chen T, Ducheyne P. The controlled release of drugs from emulsified, sol gel processed silica microspheres. Biomaterials 2009; 30:850-8.
90. Shi X, Wang Y, Ren L, Zhao N, Gong Y, Wang D A. Novel mesoporous silica-based antibiotic releasing scaffold for bone repair. Acta Biomater 2009; 5:1697-707.
91. Tuomanen E, Cozens R, Tosch W, Zak O, Tomasz A. The rate of killing of Escherichia coli by beta-lactam antibiotics is strictly proportional to the rate of bacterial growth. J Gen Microbiol 1986; 132:1297-304.
92. Gristina A G, Naylor P, Myrvik Q. Infections from biomaterials and implants: a race for the surface. Med Prog Technol 1988; 14:205-24.
93. Percival S L, Hill K E, Malic S, Thomas D W, Williams D W. Antimicrobial tolerance and the significance of persister cells in recalcitrant chronic wound biofilms. Wound Repair Regen 2011; 19:1-9.
94. Alipour M, Suntres Z E, Lafrenie R M, Omri A. Attenuation of Pseudomonas aeruginosa virulence factors and biofilms by co-encapsulation of bismuth-ethanedithiol with tobramycin in liposomes. J Antimicrob Chemother 2010; 65:684-93.
95. Hentzer M, Givskov M. Pharmacological inhibition of quorum sensing for the treatment of chronic bacterial infections. J Clin Invest 2003; 112:1300-7.
96. Irukayama-Tomobe Y, Tanaka H, Yokomizo T, Hashidate-Yoshida T, Yanagisawa M, Sakurai T. Aromatic D-amino acids act as chemoattractant factors for human leukocytes through a G protein-coupled receptor, GPR109B. Proc Natl Acad Sci USA 2009; 106:3930-4.
97. Hafeman A E, Zienkiewicz K J, Carney E, Litzner B, Stratton C, Wenke J C, Guelcher S A. Local delivery of tobramycin from injectable biodegradable polyurethane scaffolds. J Biomater Sci Polym Ed 2010; 21:95-112.
98. Brown K V, Li B, Guda T, Perrien D S, Guelcher S A, Wenke J C. Improving bone formation in a rat femur segmental defect by controlling bone morphogenetic protein-2 release. Tiss Eng Part A 2011; 17:1735-46.
99. Dumas J E, Zienkiewicz K, Tanner S A, Prieto E M, Bhattacharyya S, Guelcher S. Synthesis and Characterization of an Injectable Allograft Bone/polymer Composite Bone Void Filler with Tunable Mechanical Properties. Tis Eng Part A 2010; 16:2505-18.
100. Dumas J E, Brownbaer P B, Prieto E M, Guda T, Hale R G, Wenke J C, Guelcher S A. Injectable reactive biocomposites for bone healing in critical-size rabbit calvarial defects. Biomed Mater 2012; 7:024112.
101. Li B, Davidson J M, Guelcher S A. The effect of the local delivery of platelet-derived growth factor from reactive two-component polyurethane scaffolds on the healing in rat skin excisional wounds. Biomaterials 2009; 30:3486-94.
102. Nelson C E, Gupta M K, Adolph E J, Shannon J M, Guelcher S A, Duvall C L. Sustained local delivery of siRNA from an injectable scaffold. Biomaterials 2012; 33:1154-61.
103. Nozaki Y, Tanford C. The solubility of amino acids and two glycine peptides in aqueous ethanol and dioxane solutions. Establishment of a hydrophobicity scale. J Biol Chem 1971; 246:2211-7.
104. Liu C, Bayer A, Cosgrove S E, Daum R S, Fridkin S K, Gorwitz R J, Kaplan S L, et al. Clinical practice guidelines by the infectious diseases society of America for the treatment of methicillin-resistant Staphylococcus aureus infections in adults and children: executive summary. Clin Infect Dis 2011; 52:285-92.
105. Stewart R L, Cox J T, Volgas D, Stannard J, Duffy L, Waites K B, Chu T M. The use of a biodegradable, load-bearing scaffold as a carrier for antibiotics in an infected open fracture model. J Orthop Trauma 2010; 24:587-91.
106. Zheng Z, Yin W, Zara J N, Li W, Kwak J, Mamidi R, et al. The use of BMP-2 coupled-Nanosilver-PLGA composite grafts to induce bone repair in grossly infected segmental defects. Biomaterials 2010; 31:9293-300.
107. Wenke J C, Guelcher S A. Dual delivery of an antibiotic and a growth factor addresses both the microbiological and biological challenges of contaminated bone fractures. Exp Opin. Drug Deliv. 2011; 8:1555-69.
108. Brown K V, Walker J A, Cortez D S, Murray C K, Wenke J C. Earlier debridement and antibiotic administration decrease infection. J Surg Ortho Adv. 2010; 19:18-22.
109. Stinner D J, Noel S P, Haggard W O, Watson J T, Wenke J C. Local antibiotic delivery using tailorable chitosan sponges: the future of infection control? J Orthop Trauma. 2010 September; 24(9):592-7

We claim:

1. A composite, comprising:
a tissue graft; and
a biofilm dispersal agent on a surface of the tissue graft, within the tissue graft, or a combination thereof.

2. The composite of claim 1, wherein the tissue graft is a bone tissue graft, a skin tissue graft, or a combination thereof.

3. The composite of claim 1, wherein the tissue graft includes a polymer.

4. The composite of claim 3, wherein the polymer includes:
a polyisocyanate prepolymer, the polyisocyanate prepolymer including a first polyol and a polyisocyanate; and
a second polyol.

5. The graft of claim 4, wherein the first polyol includes poly(ethylene glycol) (PEG).

6. The graft of claim 4, wherein the polyisocyanate includes an aliphatic polyisocyanate chosen from the group consisting of lysine methyl ester diisocyanate (LDI), lysine triisocyanate (LTI), 1,4-diisocyanatobutane (BDI), hexamethylene diisocyanate (HDI), dimers and trimers of HDI, and combinations thereof.

7. The composite of claim 1, wherein the tissue graft includes a collagen sponge.

8. The composite of claim 1, wherein the tissue graft includes a tissue allograft, a tissue autograft, a tissue xenograft, a tissue isograft, a synthetic tissue substitute, or a combination thereof.

9. The composite of claim 1, wherein the tissue graft includes demineralized bone particles, mineralized bone particles, or a combination thereof.

10. The graft of claim 1, wherein the biofilm dispersal agent is selected from the group consisting of a D-amino acid, a polyamine, a recombinant DNase, a bismuth thiol, a fatty acid, cis-2-decenoic acid, tetradecanoic acid, 9-hexadecenoic acid, palmic acid, 9,12-linoleic acid, 9-oleic acid, 10-oleic acid, octadecoic acid, 7,10-oleic acid, 5,8,11,14-arachidonic acid, 7,10,13-eicosatrienoic acid, and combinations thereof.

11. The graft of claim 1, wherein the biofilm dispersal agent is a D-amino acid.

12. The graft of claim 11, wherein the D-amino acid is selected from the group consisting of D-arginine, D-histidine, D-lysine, D-aspartic acid, D-glutamic acid, D-serine, D-threonine, D-asparagine, D-glutamine, D-cysteine, D-proline, D-alanine, D-valine, D-isoleucine, D-leucine, D-methionine, D-phenylalanine, D-tyrosine, D-tryptophan, and combinations thereof.

13. The graft of claim 1, wherein the biofilm dispersal agent includes at least two of D-phenylalanine, D-methionine, D-tryptophan, and D-proline.

14. The graft of claim 1, wherein about 0.001 wt % to about 20 wt % of the composite is comprised of the biofilm dispersal agent.

15. The graft of claim 1, further comprising a biologically active agent.

16. The graft of claim 15, wherein the biologically active agent is selected from the group consisting of enzymes, organic catalysts, ribozymes, organometallics, proteins, glycoproteins, peptides, polyamino acids, antibodies, nucleic acids, steroidal molecules, antibiotics, antivirals, antimycotics, anticancer agents, analgesic agents, antirejection agents, immunosuppressants, cytokines, carbohydrates, oleophobics, lipids, extracellular matrix and/or its individual components, demineralized bone matrix, pharmaceuticals, chemotherapeutics, cells, viruses, virenos, virus vectors, prions, and combinations thereof.

17. The graft of claim 15, wherein the biologically active agent is selected from the group consisting of clindamycin, cefazolin, oxacillin, rifampin, trimethoprim/sulfamethoxazole, vancomycin, ceftazadime, ciprofloxacin, colistin, imipenem, and combinations thereof.

18. A method of treating tissue of a subject, comprising:
contacting a tissue site of a subject in need thereof with a composite, the composite including:
a tissue graft, and
a biofilm dispersal agent on a surface of the tissue graft, within the tissue graft, or a combination thereof.

19. The method of claim 18, wherein the composite releases the biofilm dispersal agent for up to 8 weeks.

20. The method of claim 18, wherein the tissue graft is a bone tissue graft, a skin tissue graft, or a combination thereof.

21. The method of claim 18, wherein the tissue graft includes demineralized bone particles.

22. The method of claim 18, wherein the tissue graft includes a polymer that comprises a polyisocyanate prepolymer, the polyisocyanate prepolymer including a first polyol and a polyisocyanate, and a second polyol.

23. The method of claim 18, wherein the biofilm dispersal agent is selected from the group consisting of a D-amino acid, a polyamine, a recombinant DNase, a bismuth thiol, a fatty acid, cis-2-decenoic acid, tetradecanoic acid, 9-hexadecenoic acid, palmic acid, 9,12-linoleic acid, 9-oleic acid, 10-oleic acid, octadecoic acid, 7,10-oleic acid, 5,8,11,14-arachidonic acid, 7,10,13-eicosatrienoic acid, and combinations thereof.

24. The method of claim 18, wherein the biofilm dispersal agent is a D-amino acid selected from the group consisting of D-arginine, D-histidine, D-lysine, D-aspartic acid, D-glutamic acid, D-serine, D-threonine, D-asparagine, D-glutamine, D-cysteine, D-proline, D-alanine, D-valine, D-isoleucine, D-leucine, D-methionine, D-phenylalanine, D-tyrosine, D-tryptophan, and combinations thereof.

25. The method of claim 18, wherein about 0.001 wt % to about 20 wt % of the composite is comprised of the biofilm dispersal agent.

26. The method of claim 18, wherein the composite further comprises a biologically active agent.

27. The method of claim 18, wherein the tissue site is a bone tissue site, a soft tissue site, or a combination thereof.

28. A method of manufacturing a composite, comprising:
providing a tissue graft; and
applying a biofilm dispersal agent on a surface of the tissue graft, within the tissue graft, or a combination thereof.

29. The method of claim 28, wherein:
the tissue graft is a polymeric material, and
the step of applying the biofilm dispersal agent includes curing a mixture of the polymeric material and the biofilm dispersal agent.

30. The method of claim 29, wherein the biofilm dispersal agent is a powder.

31. The method of claim 28, wherein the biofilm dispersal agent is selected from the group consisting of a D-amino acid, a polyamine, a recombinant DNase, a bismuth thiol, a fatty acid, cis-2-decenoic acid, tetradecanoic acid, 9-hexadecenoic acid, palmic acid, 9,12-linoleic acid, 9-oleic acid, 10-oleic acid, octadecoic acid, 7,10-oleic acid, 5,8,11,14-arachidonic acid, 7,10,13-eicosatrienoic acid, and combinations thereof.

32. The method of claim 28, wherein the biofilm dispersal agent is a D-amino acid selected from the group consisting of D-arginine, D-histidine, D-lysine, D-aspartic acid, D-glutamic acid, D-serine, D-threonine, D-asparagine, D-glutamine, D-cysteine, D-proline, D-alanine, D-valine, D-isoleucine, D-leucine, D-methionine, D-phenylalanine, D-tyrosine, D-tryptophan, and combinations thereof.

33. The method of claim 28, wherein about 0.001 wt % to about 20 wt % of the composite is comprised of the biofilm dispersal agent.

34. The method of claim 28, wherein the tissue graft is a bone tissue graft, a skin tissue graft, or a combination thereof.

35. The method of claim 28, wherein the tissue graft includes demineralized bone particles.

36. The method of claim 28, wherein the tissue graft includes a polymer that comprises a polyisocyanate prepolymer, the polyisocyanate prepolymer including a first polyol and a polyisocyanate, and a second polyol.

37. The method of claim 28, further comprising applying a biologically active agent to the tissue graft.
